(19) United States
(12) Patent Application Publication
(10) Pub. No.: US 2026/0357341 A1
(43) Pub. Date: Nov. 12, 2026

(54) RAPID CHROMOSOME SCORING

(71) Applicant: DIMENSION GENOMICS INC, San Diego, CA (US)

(72) Inventors: William K. RIDGEWAY, Encinitas, CA (US); Han CAO, San Diego, CA (US); Michael David AUSTIN, San Diego, CA (US); Jingjing GONG, San Diego, CA (US); Reha ABBASI, San Diego, CA (US)

(73) Assignee: DIMENSION GENOMICS, INC, San Diego, CA (US)

(21) Appl. No.: 19/134,395

(22) PCT Filed: Nov. 30, 2023
(86) PCT No.: PCT/US2023/081791

§ 371 Date: May 30, 2025

Related U.S. Application Data
(60) Provisional application No. 63/385,766, filed on Dec. 1, 2022.

(51) Int. Cl. C12Q 1/6876; B01L 3/00; C12Q 1/04; C12Q 1/25; C12Q 1/6806; G16B 30/10

(52) U.S. Cl. CPC C12Q 1/6876; B01L 3/5027; C12Q 1/04; C12Q 1/25; C12Q 1/6806; G16B 30/10; B01L 2300/161; B01L 2400/0406; C12Q 2600/156; C12Q 2600/16; G01N 2800/385; G01N 2800/7028

(57) ABSTRACT

Rapid, accurate, high throughput automated cytological analysis technologies are disclosed herein. Through these approaches, tens or hundreds of chromosomes or more may be analyzed in an automated fashion. These approaches allow rapid identification and quantification of chromosomal number, chromosome aberrations, identity and number of pathogen nucleic acids in a sample.

### CROSS-REFERENCE TO RELATED APPLICATIONS

This document claims priority to U.S. Provisional Ser. No. 63/385,766, filed Dec. 1, 2022, the contents of which are hereby incorporated by reference in their entirety.

### FIELD OF THE INVENTION

This disclosure relates to assays of biological molecules, and more particularly to assays for rapidly determining genomic information from a large number of chromosomes.

### BACKGROUND

Cytogenetic analysis of intact chromosomes has had a profound impact on the study of human genetics, both of healthy and abnormal individuals. Numerical abnormalities such as additional copies of entire chromosomes and finer scale lesions such as insertions and deletions of portions of chromosomes have underpinned patient diagnoses and guided genetic research into the underlying abnormalities. The subsequent development of FISH (Fluorescence In Situ Hybridization) in the early 1980s allowed for higher resolution probing of specific genomic loci by leveraging sequence specific recognition of probes. A growing understanding of the role of aneuploidies in cancer and the discovery of extra-chromosomal DNA and its role in human disease both benefit heavily from cytogenetic methods for basic discovery and medical diagnostics.

High throughput short read sequencing has complemented cytogenetic methods but as useful as it is, has not replaced cytogenetic analysis. There are fundamental challenges to undo the destructive nature of the sample preparation. Considerable effort is needed in terms of sample barcoding and preparation and downstream bioinformatic analysis in order for short read sequencing to obtain long-range genomic information from a sample.

Cytogenetic analysis involves lysis of a cell or cell population and deposition of the chromosomes onto a microscope slide for visual characterization by a technician. The process is often subjective and reliant on the expertise of the technician, both in depositing the chromosomes so that they are distinct, nonoverlapping and not folded so that they may be readily visualized, and in identifying the deposited chromosomes and spotting potential defects or abnormalities.

This analysis as currently practiced does not scale and is very expensive and laborious. Routine sample preparation requires expert judgment to tune and refine preparations. Experts are again needed to interpret subtle differences in images and make diagnoses. Consequently, routine analysis typically is only able to analyze 20 or so cells from a patient, and it is common for each of these 20 cells to contain one or more unobservable or uncharacterizable chromosomes due to overlap with adjacent chromosomes.

There is an unmet need for a method to scale cytogenetic analysis in order to observe a large number of chromosomes at once. By increasing the number of chromosomes observed and thus increasing the depth of coverage, deep coverage data contain the statistical power to capture rare events such as a few cancerous cells in a population of healthy cells. The present invention addresses this need and conveys additional benefits.

### SUMMARY

Disclosed herein are systems, compositions and methods related to the deposition of nuclei acid molecules onto a surface, such as a solid surface, and to the systems resultant from said deposition. The deposition in some cases facilitates rapid imaging of the nucleic acid molecules, such as for high throughput characterization of the molecules. The surface may comprise a high frequency or abundance of non-overlapping nucleic acids, such as at least 10,000, and may further comprise overlapping nucleic acid molecules at a frequency of no more than 10%, 5%, 2%, 1%, 0.5%. 0.2%, 0.1% or less. The molecules may be unmodified nucleic acids, as large as intact chromosomes, and may be synthesized in vivo within cells or naturally occurring. The nucleic acids may be deposited without being covalently tethered to the surface. In some cases, the molecules are similarly not tethered through base paring to probes or other oligos covalently tethered to the surface. The nucleic acid molecules in some cases comprise basepairs having a density of nucleic acid basepairs of at least 100 Gb/mm2 on the surface. Some populations of nucleic acids on the surface, such as the at least 10,000 non-overlapping nucleic acid molecules, have a median length of at least 100 kb.

Through practice of the disclosure herein, one may obtain a surface having deposited thereon unmodified nucleic acids-that is, nucleic acids that in some cases have not bad probe binder, adapter or library packaging modifications, or other covalent modification added to them-such that no more than 1% of the nucleic acid molecules overlap at least one adjacent nucleic acid molecule. The systems variously comprise at least 10,000 molecules, at least 20,000 molecules, at least 50,000 molecules, at least 100,000 molecules, at least 200,000 molecules, at least 500,000 molecules, or at least 1,000,000 molecules or more.

Similarly, some or all of the counted nucleic acids on the surface, which may further comprise smaller molecules not included in the nucleic acid ‘count’ on the surface, comprise at least 10,000 base pairs, at least 20,000 base pairs, at least 50,000 base pairs, at least 100,000 base pairs, at least 200,000 base pairs, at least 500,000 base pairs, or at least 1,000,000 base pairs or more.

The nucleic acid molecules are often deposited as nucleic acid protein complexes, for example configured as chromatin, comprising both a nucleic acid molecule and chromatin-associated proteins such as histone proteins H2A, H2B, H3 or H4 alone or assembled into nucleosomes, Histone H1, condensin 1, condensin 2, or other chromatin constituent or chromatin associated proteins, or other proteins. In some cases, the nucleic acids comprise at least 25%, at least 50%, at least 60%, at least 70%, at least 80%, at least 90%, at least 95% or greater than 95% of the protein or chromatin constituents of a native chromosome. In some cases, the nucleic acids are deposited as condensed chromatin complexes, such as metaphase chromosomes.

The nucleic acid molecules are deposited in some cases as components of a processed aggregate solution rather than maintaining cellular registration. That is, adjacent chromosomes or nucleic acid molecules bound or unbound by chromatin or other components do not imply a common source cell in the systems and methods herein, in contrast to approaches in the art.

The nucleic acids variously comprise human DNA such as human host DNA, fetal DNA, paternal DNA, maternal DNA, extrachromosomal human DNA, pathogen DNA, bacterial DNA such as packaged bacterial DNA from bacterial chromosomes or plasmids, viral DNA such as packaged viral DNA or other DNA from a sample such as a human sample or other organism or environmental sample, particularly DNA having some secondary or tertiary structure arising from packaging molecule such as chromatin constituents bound thereto.

The nucleic acids, modified or unmodified, are in some cases bound to a label or labels, such as a label that is specific to a nucleic acid binding component or to a specific nucleic acid such as a specific chromosome or portion of a chromosome. Labels variously comprise fluorescent labels, oligo probes, epitopes or epitope binding moieties such as antibodies, for example antibodies that target a particular chromatin constituent or a particular modification to a chromatin protein constituent or to a nucleic acid. Some labels are provided in combination, comprising for example a first label and a second label, where the first label and the second label bind, for example, a first segment and a second segment of a chromosome, or a modified and unmodified portion of chromatin, or other distinct portions of a nucleic acid or of its binding partners such as chromatin constituents. Labels variously bind to regions of high AT content, high GC content, chromatin, telomeres, centromeres, chromosomal bands either newly characterized or as described or established in the field, such as the banding patterns described in the Paris conference of 1971, or a particular target sequence, such as a sequence implicated in a chromosomal aberration such as a duplication, deletion, inversion or translocation, or a particular gene, coding or noncoding region, or regulatory element. Some labels bind to particular higher order chromatin structures. Some labels bind chromosomes in a substantially uniform manner to aid in the delineation of the gross structure of the chromosome including centromeric position.

Surfaces comprising nucleic acids variously comprise chromosomes at a proportion that is indicative of a chromosomal number abnormality such as aneuploidy in all or a subset of cells from which the nucleic acids are collected, such as fetal cells collected from a maternal sample. Surfaces comprising nucleic acids variously comprise at least one chromosome comprising a chromosomal abnormality such as a translocation, inversion, deletion or duplication. The chromosomal abnormality is in some cases present at a frequency of no greater than 1% of the nucleic acids or chromosomes on the surface, or no more than 0.1% of the nucleic acids or chromosomes on the surface, or no more than 0.0001% of the nucleic acids or chromosomes on the surface, no more than 100 of the nucleic acids or chromosomes on the surface, no more than 10 nucleic acids or chromosomes on the surface, or no more than 1 nucleic acid or chromosome on the surface.

Surfaces comprising nucleic acids variously comprise at least one viral chromosome or nucleic acid. Surfaces comprising nucleic acids variously comprise at least one bacterial (eubacterial or archaeal) chromosome or nucleic acid. Surfaces comprising nucleic acids variously comprise at least one viral chromosome or nucleic acid. Surfaces comprising nucleic acids variously comprise at least one eukaryotic pathogen chromosome or nucleic acid.

Surfaces comprising nucleic acids variously comprise at least one transgenic chromosome or nucleic acid. Surfaces comprising nucleic acids variously comprise at least one nucleic acid comprising an engineered base or a base that does not match that of a nearest wild-type representative at that position. Surfaces comprising nucleic acids variously comprise at least one CRISPR edited site in a chromosome or nucleic acid.

Surfaces consistent with the disclosure may comprise various features. Some such surfaces comprise parallel grooves having a width configured to accommodate human metaphase chromosomes in single file series. Some surfaces comprise fluidic features having dimensions configured so as to bias the positioning of chromosomes such that chromatids or centromeres are aligned relative to surface features. Some surfaces comprise fluidic features having dimensions configured so as to accommodate particular target molecules, such as human metaphase chromosomes. Similarly, some surfaces comprise wells or pits configured to accommodate particular nucleic acid molecules such as extrachromosomal DNA molecules or ecDNA.

Some systems consistent with the disclosure comprise an imaging component or interrogation component. Such a component may operate in coordination with or independent of an interrogation analysis component.

Such a component may identify a signature present in all or a subset of nucleic acid molecules interrogated on the surface of the system. The signature is in some cases associated with a disease such as cancer, or with a phenotype, an inherited trait, a mutation, a non-inherited trait, a variant, such as a structural variant, or an SNP. The signature is in some cases associated with an infection organism, an invading organism, a pathogen, a parasite, a symbiotic organism, a host organism, a metagenomic profile, or a particular microorganism. The signature is in some cases associated with a type of chromosome, ecDNA, a circular nucleic acid, a type of cell such as a circulating tumor cell, or of genetic information.

Signatures variously comprise a banding pattern of a nucleic acid, particularly assembled into a chromosome, or a label, a physical map, a physical dimension of a nucleic acid or chromosome, a physical mass, morphology or topology, a chromosome arm length.

Identification of a signature in some cases comprises high throughput analysis of the nucleic acids or chromosomes on the surface. Analysis may comprise assessing at least 10,000 molecules, at least 20,000 molecules, at least 50,000 molecules, at least 100,000 molecules, at least 200,000 molecules, at least 500,000 molecules, or at least 1,000,000 molecules or more. Assessing is in some cases completed in no more than 60, 30, 15, 10, 5 or one minute after capture. Assessing is in some cases completed in no more than 60, 30, 15, 10, 5 or one minute after initiating sample preparation. Assessing is in some cases completed in no more than 60, 30, 15, 10, 5 or one minute after contacting a sample to the surface.

Interrogating is in some cases accomplished at a rate of at least 100 chromosomes per minute, or variously 200 chromosomes per minute, 500 chromosomes per minute, 1000 chromosomes per minute, or more.

Identification of a signature in some cases comprises comparison to a reference, such as alignment to a reference.

To facilitate analysis, in some cases the nucleic acid molecules are deposited on the surface at a density of at least one per 500 microns square, or in some cases, one per 400 microns square, one per 300 microns square, one per 200 microns square, one per 100 microns square, one per 50, microns square, one per 25 microns square, one per 10 microns square or less.

The system in some cases comprises a fluidic device, such as a fluidic device that provides or removes or provides and removes a liquid from the surface, for example a liquid comprising nucleic acids or chromosomes from a sample. The surface in some cases comprises fluidic channels or fluidic features, which in some cases facilitate nucleic acid molecule arrangement on the surface.

Nucleic acid molecules or chromosomes are positioned through a number of approaches consistent with the disclosure herein. Some positioning methods comprise deposition. Some deposition approaches comprise deposition onto the surface by first depositing the sample as a liquid and then employing a receding meniscus process to leave nucleic acid molecules deposited on the surface. Receding meniscus approaches consistent with the disclosure herein variously comprise blade coating, combing, or dip coating.

Alternately or in combination, deposition is effected through use of centripetal force, dispensing, or fluid flow, such as capillary flow through a fluidic device such as an open fluidic device. Some positioning methods comprise transfer printing.

A fluidic device may comprise fluidic features, such that nucleic acid molecules are preferentially deposited within the fluidic features. The fluidic features are in some cases sized to accommodate a nucleic acid molecule or chromosome or nucleic acid molecule harboring binding moieties such as exogenously added binding moieties or chromosome constituents of a certain identity, such as human chromosome 1, 2,3, 4, 5, 6, 7, 8, 9, 10, 11, 12, 13, 14, 15, 16, 17, 18, 19, 20, 21, 22, X or Y, either wild type or harboring one or more insertions, deletions, duplications, translocations, inversions, or other known or predicted structural permutations, such as may be associated with a condition such as cancer, or one or more ecDNA molecules. In some cases, fluidic features have a long axis selected to accommodate a target based upon the target's expected length along its long axis. Some such axes are selected, for example to accommodate human chromosomes or human ecDNA.

The fluidic features variously comprise topological elements, pillars, or channels, for example, such as those having a dimension spanning from 0.1 to 10 microns. The dimension variously comprises a width, length, depth, height or cross-section. In some cases, the fluidic features comprise a dimension comparable to that of the target molecule. The target molecule may in some cases be positioned between or within a field of pillars, particularly pillars situated so as to have a separation distance selected to accommodate the target molecule. In some cases, the dimension is a diameter.

Alternately, a fluidic feature mat comprises a region having a surface property, such as being differentially hydrophobic, hydrophilic, having anti-fouling capacity, or roughness such as roughness greater than or equal to or less than 50 nm rms, such as 10, 20, 30, 40, 50, 60, 70, 80, 90, or 100. Features may be selected to configure a molecule withing the feature or to exclude a molecule from the feature.

Examples of such features include a material such as a monolayer, a film, a polymer, a channel, such as one having a porous roof, such that a molecule may or may not transit the porous roof. Some features may lead to molecules being positioned preferentially at regions elevated relative to the surface, or depressed relative to the surface.

The systems disclosed above further comprise reagents or facilitate methods comprising the application of reagents to nucleic acid molecules of the surface. The application of such a reagent or reagent may lead to the nucleic acid molecule being further processed, such as while it is positioned on the surface. Such a reagent may comprise a gel, a liquid, a solution, a dissolved polymer, and may comprise an enzyme. Similarly, the methods and compositions may comprise exposing at least one nucleic acid molecule or other molecule to a reagent, such as an enzyme, oligo, fluorophore, or binding protein. The systems or methods may comprise exposure to an environmental condition, exposing or subjecting to at least one chemical reaction, or binding at least one body to the nucleic acid molecule or other target molecule. Processing variously comprises in some cases nicking, denaturing, cleaving, binding or otherwise interacting with a molecule of the surface. Application is effected through, in some cases, a deposition system, a contact probe system or an AFM.

Consistent with the systems and methods above and throughout the present disclosure, disclosed herein are surfaces, such as surfaces for accommodation of nucleic acid molecules alone or bound in chromosomes or bound to binding partners. Said surfaces are in some cases unmodified, while in other cases said surfaces comprise fluidic features such as parallel grooves. Surfaces may comprise at least or no more than 10, 20, 50, 100, 200, 500, 1,000, 2,000, 5,000, 10,000 or more than 10,000 such grooves. Grooves are in some cases configured to accommodate single file linear arrays of chromosomes, or alternately to accommodate single chromosomes or other nucleic acid complexes, such as human chromosome 1, 2,3, 4, 5, 6, 7, 8, 9, 10, 11, 12, 13, 14, 15, 16, 17, 18, 19, 20, 21, 22, X or Y, either wild type or harboring one or more insertions, deletions, duplications, translocations, inversions, or other known or predicted structural permutations, such as may be associated with a condition such as cancer, or one or more ecDNA molecules. In some cases, fluidic features have a long axis selected to accommodate a target based upon the target's expected length along its long axis. Some such axes are selected, for example to accommodate human chromosomes or human ecDNA.

Concurrent with disclosure of the systems comprising nonoverlapping nucleic acid molecules on surfaces and related systems, images, and methods, disclosed herein are compositions and methods for nucleic acid or condensed chromosome preparation, such as in expectation of deposition on a surface herein or use in a system herein.

Accordingly, disclosed herein are methods of enriching for condensed chromosomes. Many such methods comprise lysing a population of cells so as to produce a lysate comprising unlysed nuclei and free cytosol contents, and discarding nuclei form the lysate, thereby enriching for condensed chromosomes from anucleate cells. In some cases, the anucleate cells are cells in M phase or otherwise comprise condensed chromosomes, such as condensed chromosomes not contained within nuclei.

The population of cells is in some cases cultured, such as in contact with growth factors that allow the cells to progress through at least one phase of a eukaryotic cell cycle prior to lysing. The cells are in some cases cultured in contact with a cell cycle progression inhibitor, such as a cell cycle inhibitor that blocks cell cycle progression at M phase.

Exemplary inhibitors comprise a colcamid, or a cell cycle inhibitor such as resveratrol or lambersulfat, although additional cell cycle progression inhibitors that block cell cycle progression at or near M phase are also contemplated.

Alternately, cells are cultured in some cases under conditions that promote cell cycle synchronization, such as synchronization at M phase. Synchronization is effected, for example, by nutrient removal, temperature shock, or contacting with a small molecule cell cycle progression inhibitor.

Lysis is effected through any number of approaches consistent with the disclosure herein. In some cases, a lysis approach is selected to as to breach the cytoplasmic or cellular membrane phospholipid bilayer without disrupting or without substantially either the nuclear membrane or the assemblage of proteins bound to nucleic acid molecules in condensed chromosomes. Exemplary lysis approaches comprise generation of an osmotic gradient across cell membranes, or contacting the cell to an enzyme such a lytic enzyme or a phospholipid bilayer degrading enzyme.

Nuclei are discarded through any number of approaches consistent with the disclosure herein. In some cases, a nuclei clearance approach is selected to as to remove nuclei without disrupting or without substantially either the nuclear membrane or the assemblage of proteins bound to nucleic acid molecules in condensed chromosomes. Some exemplary approaches comprise centrifugation, immunoprecipitation or immunobead-based separation as effected by nuclei binding magnetic beads, or sedimentation.

Condensed chromosomes are in some cases added directly to a surface or otherwise used in or to form a system as disclosed herein. Alternately, condensed chromatin or other anucleate lysate products are contacted to a detergent, such as a detergent that coats nucleic acids or chromatin.

Similarly, condensed chromatin or other anucleate lysate products are in some cases contacted to or resuspended in a deposition buffer. Some exemplary deposition buffers exhibit one or more of the following properties: they are volatile at room temperature; they maintain or do not substantially disrupt integrity of the enriched condensed chromosomes, for example such that some or all of the chromatin constituents such as nucleosomes, histones H2A, H2B, H3, H4, or H1 condensin 1 or condensing 2 remains at least partially bound to the nucleic acid molecule of the chromosome; they comprising stabilizing salts, or they are acidic. Some exemplary deposition buffers have a pH of no greater than 4, 3, 2 or less than 2. Some exemplary buffers have a pH of about 2 or 2.

In some cases, the cell lysates are not substantially processed so as to create or introduce phosphodiester bonds, such as would be required to prepare the nucleic acids for deposition onto a flow cell for sequencing. That is, adapters such as P5 or P7 adapters or other adapters used in attaching nucleic acid libraries to flow cell surfaces pursuant to bridge amplification, or other sequencing adapters are not added to the nucleic acid molecules of the lysate. Similarly, the lysate is not processed to be compatible with long read sequencing, such as by formation of circular ‘smart bell’ molecules as used in some long read sequencing approaches.

Cell lysates are deposited onto a surface, such as a surface disclosed herein or otherwise consistent with the disclosure herein. Exemplary surfaces comprise, for example grooves configured to accommodate human metaphase or condensed chromosomes in single file lines along their long axes. Said surfaces are in some cases unmodified, while in other cases said surfaces comprise fluidic features such as parallel grooves. Surfaces may comprise at least or no more than 10, 20, 50, 100, 200, 500, 1,000, 2,000, 5,000, 10,000 or more than 10,000 such grooves. Grooves are in some cases configured to accommodate single file linear arrays of chromosomes, or alternately to accommodate single chromosomes or other nucleic acid complexes, such as human chromosome 1, 2,3, 4, 5, 6, 7, 8, 9, 10, 11, 12, 13, 14, 15, 16, 17, 18, 19, 20, 21, 22, X or Y, either wild type or harboring one or more insertions, deletions, duplications, translocations, inversions, or other known or predicted structural permutations, such as may be associated with a condition such as cancer, or one or more ecDNA molecules. In some cases, fluidic features have a long axis selected to accommodate a target based upon the target's expected length along its long axis. Some such axes are selected, for example to accommodate human chromosomes or human ecDNA.

Lysates are deposited such that the resulting surface comprises overlapping nucleic acid molecules or overlapping chromosomes at a frequency of no more than 1%. In some cases the nucleic acid molecules are deposited on the surface at a density of at least one per 500 microns square, or in some cases, one per 400 microns square, one per 300 microns square, one per 200 microns square, one per 100 microns square, one per 50, microns square, one per 25 microns square, one per 10 microns square or less.

Consistent with the methods herein, disclosed are compositions comprising intact chromosomes unbound by nuclei, a cell cycle progression inhibitor, a detergent, and a volatile solvent at an acidic pH. The compositions comprise in some cases at least 10,000, 20,000, 50,000, 100,000, 200,000, 500,000, 1,000,000 or more intact chromosomes. Intact chromosomes comprise in some cases one or more of nucleosomes histone H2A, Histone H2B, histone H3, histone H4, histone H1, condensing 1 or condensing 2 at native concentrations or at concentration of at least 99%, 95%, 90%, 75%, 50%, or 25% that of native concentrations.

Exemplary cell cycle inhibitors include a colcamid, resveratrol or lambersulfat, although additional cell cycle progression inhibitors that block cell cycle progression at or near M phase are also contemplated. Cell cycle inhibitors are often present at a low concentration consistent with their being present in grow medium and taken up by cells during cell cycle synchronization prior to cell lysis.

A number of detergents and volatile solvents are consistent with the disclosure herein and are disclosed in more detail in the section below. Some exemplary deposition buffers have a pH of no greater than 4, 3, 2 or less than 2. Some exemplary buffers have a pH of about 2 or 2.

Also disclosed herein are methods of analyzing chromosomes in a sample. Some such methods comprise one or more of depositing chromosomes on a surface such that at least 10,000 chromosomes are deposited and no more than 1% of the chromosomes overlap.

Alternately, methods may comprise using any system as disclosed herein comprising chromosomes or nucleic acids on a surface. The methods further comprise interrogating the surface comprising the chromosomes to create an image, and performing an automated identification of the chromosomes in the image.

In some cases, the methods comprise interrogating at least 10,000, 20,000, 50,000, 100,000, 200,000, 500,000, 1,000,000 or more intact chromosomes on a surface, of which no more than 1%. Or no more than 0.1%, or no more than 0.001% exhibit overlap.

In exemplary embodiments the chromosomes comprise condensed metaphase chromosomes, such as human host DNA, fetal DNA, paternal DNA, pathogen DNA, viral DNA.

Chromosomes may be unlabeled or bound to at least one label, such as a fluorescent label, comprising nucleic acid oligos, epitopes or epitope binding regions. Some labels comprise a first label that binds a first chromosomal segment and a second label that binds a second chromosomal segment.

Chromosomes are in some cases present in a proportion that is indicative of a fetal chromosome number abnormality. For example, some methods relate to identifying a fetal chromosomal number abnormality when a first chromosome population is present at an amount that differs substantially from that of at least one second chromosomal population.

Abnormalities may be detected by evaluating relative ratios of chromosome populations or by evaluating the presence or abundance of one or more types of chromosomal abnormalities such as a structural variation, translocation, insertion, deletion, inversion or duplication. In some cases the structural variation is present at no more than 1% of the chromosomes on the surface, or no more than 0.1% of the chromosomes on the surface, or lower abundance to even being present at no more than 100 copies, no more than 50 copies, no more than 20 copies, no more than 10 copies, no more than 5 copies, no more than 2 copies, or as a single copy on the surface.

Similarly, pathogens or foreign constituents of a sample may be detected through the approaches herein. In some cases a foreign constituent is present at no more than 1% of the chromosomes on the surface, or no more than 0.1% of the chromosomes on the surface, or lower abundance to even being present at no more than 100 copies, no more than 50 copies, no more than 20 copies, no more than 10 copies, no more than 5 copies, no more than 2 copies, or as a single copy on the surface. Exemplary foreign constituents comprise viral genomes, bacterial genomes, or non-host eukaryotic chromosomes, and are in some cases identified de novo in the samples, or are quantified as to abundance so as to assess severity of infection or efficacy of a treatment, for example.

Similarly, transgenic or modified chromosome constituents of a sample may be detected through the approaches herein. In some cases a transgenic or modified chromosome constituent is present at no more than 1% of the chromosomes on the surface, or no more than 0.1% of the chromosomes on the surface, or lower abundance to even being present at no more than 100 copies, no more than 50 copies, no more than 20 copies, no more than 10 copies, no more than 5 copies, no more than 2 copies, or as a single copy on the surface. Exemplary transgenic or modified constituents comprise an engineered base, a CRISPR edited site, a homologous recombination site, a transgenic insert site, or other exogenous genetic modification.

A number of analysis approaches are consistent with the methods described herein. Some such approaches comprise sorting chromosomes identified in an image of chromosomes on a surface. The analysis is facilitated by the low rate of overlap among the chromosomes on the surface. Sorting is effected by, for example, forming a set comprising two chromosomes identified as being similar to one another on the basis of sharing a similar property. One such similar property is genomic content, as may be determined by comparing chromosomal banding patters among chromosomes in a surface or to a precharacterized chromosome set. A first and a second chromosome may be assigned to a common set if, for example, they share across their length or across segments of each chromosome, a percentage of identical genomic content, such as at least 75%, at least 80%, at least 85%, at least 90%, at least 95%, at least 99%, or 100% identical content. Identity may be assessed as an aggregate over the length of compared segments or over the length of the chromosomes, and may be assessed in terms of the percent of the chromosomes or segments sharing complete identity, or may be assessed by factoring in local variation such as SNPs that may cause local identity to fall below 100%.

Similarity may be assessed by performing an alignment of the physical maps or binding patterns of all or segments of two chromosomes. Similarity may be determined by comparing chromosomal arm lengths, end to end or from a measuring point such as centromere locations in the at least two chromosomes. As an alternative marker or measuring point, one may use existence or location of at least one labeling body.

Some comparisons or chromosome analyses comprise forming an in silico model of a chromosome or of a pair of chromosomes to be compared. The model may be formed using any of the traits used to sort chromosomes mentioned above, such as length, identify, centromere location relative to arm length, or marker location.

Analyses may further comprise identifying a third chromosome that does not group into the set comprising the first and second chromosomes based upon the criterion used to sort the first and second chromosomes together. In some cases, the third chromosome sorts with a fourth chromosome into a second set whose membership is exclusive to the first set.

Some analyses comprise comparing the first set to the second set, by for example determining a ratio of the number of chromosomes in the first set to the number of chromosomes in the second set.

Such a ratio may be indicative of a number of characteristics of a sample from which the chromosomes were obtained. For example the ratio of the number of chromosomes in the first set to the number of chromosomes in the second set may be indicative of the number of cells in the sample that harbor the chromosomes of the first set or havening the similarity property of the first set, relative to the number of cells in the sample that harbor the chromosomes of the second set or havening the similarity property of the second set.

Similarly, such a ratio may be indicative of a chromosomal number abnormality such as an aneuploidy present in some or all of the cells in the sample. In these cases, the ratio of the first set to the second set may be indicative of the relative frequency of aneuploid cells in the sample.

Some additional similarity criteria comprise chromosome number, presence of a genomic lesion, or the type of chromosome or nucleic acid molecule to which binding proteins have assembled, such as the ratio of native chromosomes to ecDNA in a sample, either in aggregate or narrowed by the use of a marker common to a chromosome type and some or all ecDNA. Alternately or in combination, similarity properties may be chromosome morphology, such as shape, arm presence or absence, or length variation, or presence of telomeres or ends as opposed to circular morphologies.

A common feature of many of the methods herein is that they comprise an automated assessment of images of the chromosomes or nucleic acid molecule bound complexes on a surface. This feature represents a major advantage over current cytological analysis, which is heavily reliant upon human skill in delivering samples to surfaces and human judgement in scoring them. In some methods herein, automated identification does not comprise human assessment of the image prior to scoring. Automated assessment is completed, in some cases in no more than 6 hours, 1 hour, 30 minutes, 15 minutes, 10 minutes, 5 minutes 1 minute or less than one minute. Some approaches further comprise human verification of scored sample images.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

For all drawings, the use of roman numerals: i), ii), iii), iv), etc are to denote a passage of time. Unless specifically stated, the figures are not drawn to scale.

FIG. 1 demonstrates different, non-limiting embodiments of confined and non-confined channel types within a fluidic device.

FIG. 2 demonstrates different, non-limiting embodiments of patterning surface properties on a substrate.

FIG. 3 demonstrates a fluorescent image of chromosomes ordered by deposition onto a sawtooth ridge grating pattern of a fluidic device, where the periodicity of the ridges is 3.33 microns. The device was fabricated in PDMS by replication of a blazed optical grating. Chromosomes are packed at an inverse density of 15um2 /chromosome and a basepair density of 8.5 Mb/um2.

FIG. 4 demonstrates a fluorescent image of chromosomes positioned in channels of a fluidic device.

FIG. 5 demonstrates a fluorescent image of chromosomes positioned in channels of a fluidic device.

FIG. 6 demonstrates a fluorescent image of chromosomes positioned in channels of a fluidic device.

FIG. 7 demonstrates a fluorescent image of chromosomes flowing into a fluidic device (from bottom to top of the image) by capillary flow. In this particular embodiment, the substrate is silicon, and the features include channels approximately 2 micron wide.

FIG. 8 demonstrates a fluorescent image of positioned chromosomes in a fluidic device after capillary loading. The device comprises fluidic features including topological features and patterned surface modification features.

FIG. 9 demonstrates an embodiment of positioning chromosomes on the surface of a fluidic device by blade coating, whereby the device comprises fluidic features including topological features and patterned surface modification features.

FIG. 10 demonstrates an embodiment of positioning chromosomes on the surface of a fluidic device by blade coating, whereby the device comprises fluidic features including topological features.

FIG. 11 demonstrates an embodiment of positioning chromosomes on the surface of a fluidic device by dip coating, whereby the device comprises fluidic features including topological features and patterned surface modification features.

FIG. 12 demonstrates an embodiment of positioning chromosomes on the surface of a fluidic device by flow-cell coating, whereby the device comprises fluidic features including topological features and patterned surface modification features.

FIG. 13 demonstrates an embodiment of positioning chromosomes on the surface of a fluidic device by flowing whereby the device comprises fluidic features including topological features and patterned surface modification features.

FIG. 14 demonstrates an embodiment of positioning chromosomes on the surface of a fluidic device by flowing, whereby the device comprises fluidic features including closed channels, and a removable roof.

FIG. 15 demonstrates an embodiment of positioning chromosomes on the surface of a substrate by spin coating.

FIG. 16 demonstrates an embodiment of positioning chromosomes on the surface of a fluidic device by dispensing, whereby the device comprises fluidic features including topological features and patterned surface modification features.

FIG. 17 demonstrates an embodiment of positioning chromosomes on the surface of a fluidic device by dispensing, whereby the device comprises fluidic features including a porous roof over a fluidic channel.

FIG. 18 demonstrates an embodiment for manufacturing a fluidic device with a porous roof over a fluidic channel.

FIG. 19 demonstrates an embodiment of positioning chromosomes on the surface of a substrate by contract printing the chromosomes with an intermediate substrate.

FIG. 20 demonstrates a scanning electron microscope image of an embodiment of fluidic features for a fluidic device. In this particular embodiment, the substrate is silicon, and the features include channels approximately 2 micron wide, and 1 micron deep.

FIG. 21 demonstrates a scanning electron microscope image of an embodiment of fluidic features for a fluidic device. In this particular embodiment, the substrate is silicon, and the features include pillars approximately 1 micron long, 250 nm wide, and 1 micron tall.

FIG. 22 demonstrates a scanning electron microscope image of an embodiment of fluidic features for a fluidic device. In this particular embodiment, the substrate is silicon, and the features include pits approximately 800 nm wide, and 1 micron deep.

FIG. 23 demonstrates a scanning electron microscope image of an embodiment of fluidic features for a fluidic device. In this particular embodiment, the substrate is silicon, and the features include pits approximately 200 nm wide, and 1 micron deep.

FIG. 24 demonstrates a scanning electron microscope image of an embodiment of fluidic features for a fluidic device. In this particular embodiment, the substrate is silicon, and the features include pillars approximately 500 nm wide, and 1 micron tall

FIG. 25 demonstrates a fluorescent image of chromosomes physically confined within a 3 micron wide channel, and pushed up against a filter region whereby the channel width is reduced to 1 micron wide, preventing the chromosomes from progressing further (from right to left in the image) by physical obstruction.

FIG. 26 demonstrates a fluorescent image of chromosomes and ecDNA positioned in 3 micron wide channels of a fluidic device, whereby the chromosomes and ecDNA physically position themselves differently due to their respective physical size difference. The chromosomes span the width of the channel, contacting both sides, while the ecDNAs are drawn into the corners of the channels.

FIG. 27 demonstrates a fluorescent image of positioned chromosomes in channels of a fluidic device, whereby chromosomes containing a certain sequence associated with the BCR-ABL translocation are bound to with FISH probes.

FIG. 28 demonstrates a fluorescent image of chromosomes positioned in channels of a fluidic device, whereby the sub-micron wide channels sufficiently confine the chromosomes to enable elongation of the chromosomes along the lengths of the sub-micron channels.

FIG. 29 demonstrates a fluorescent image of chromosomes positioned in channels of a fluidic device, whereby the 2.5 micron wide channels are appropriately spaced to enable the chromatids to be physically separated for interrogation of the centromeres.

FIG. 30 demonstrates a fluorescent image of chromosomes positioned in channels of a fluidic device, whereby the 2.5 micron wide channels are appropriately spaced to enable the chromatids to be physically separated.

FIG. 31 demonstrates a fluorescent image of chromosomes positioned in channels of a fluidic device, whereby the centromere of the chromosomes is positioned on the bar that separates adjacent fluidic channels.

FIG. 32 demonstrates an embodiment of positioning banded chromosomes in channels of a fluidic device such that multiple chromosomes can be interrogated in parallel. In this particular drawn embodiment, chromosome #10 is identified multiple times by interrogation, and an abnormal version of chromosome #10 is also identified.

FIG. 33 An enclosed fluidic device that contains a patterned surface upon which chromosomes are ultimately deposited for interrogation 335, in addition to other fluidic features that restrict the number of cells that fit in the cell chamber 333, allow cells to be washed and manipulated via a frit or blocking pillar array 334, but which is small enough to allow intact chromosomes to move from cell chamber 333 to interrogation chamber 335. The second frit or pillar array 336 is smaller than 334, and is able to block intact chromosomes from exiting the device via outlet 332.

FIG. 34 Manipulations of a fluidic device that contains a surface upon which chromosomes are ultimately deposited in addition to a chamber that facilitates the processing of cells and purification of nuclei away from condensed chromosomes.

FIG. 35 demonstrates a bright field image of banded chromosomes positioned on a non-patterned surface of a glass substrate. Chromosomes are G-banded trypsin using Giemsa (GtG).

FIG. 36 demonstrates a bright-field image of banded chromosomes positioned within channels of a fluidic device. Chromosomes are G-banded trypsin using Giemsa (GtG).

FIG. 37 demonstrates a bright-field image of banded chromosomes positioned within channels of a fluidic device. Chromosomes are G-banded trypsin using Giemsa (GtG). Here, chromosome are expanded in the channel by long incubation in trypsin.

FIG. 38 demonstrates a bright field image of banded chromosomes positioned on a non-patterned surface of a glass substrate Chromosomes isolated from K562 cells, deposited onto an un-structured glass surface, stained with Giemsa banding with trypsin and imaged under brightfield illumination. Chromosomes are present on glass at an inverse density of 50 um2/chromosome and a basepair density of 2.4 Mb/um2 .

FIG. 39 demonstrates a fluorescent image of chromosomes on a non-patterned substrate. Chromosomes isolated from K562 cells, deposited onto an un-structured glass surface, stained with YOYO-1 and imaged using a fluorescent microscope.

FIG. 40 demonstrates a bright field image of a typical spread of banded chromosomes from a single cell generated by traditional cytogenetic methods of dropping a solution of cells on a glass slide.

### DETAILED DESCRIPTION

The practice of the techniques described herein may employ, unless otherwise indicated, conventional techniques and descriptions of cytogenetics, chemistry, biochemistry, electrical engineering (as pertains to microfabrication), optics, photophysics, biophysics, and bioinformatics, which are within the skill of those who practice in the art. Such conventional techniques include preparation of chromosomal spreads, surface chemical modifications ordered deposition of micron scale particles, nucleic acid hybridization, biochemical staining, fluorescence and transmitted light microscopy, quantitative image analysis and bioinformatic analysis. Specific illustrations of suitable techniques can be had by reference to the examples herein. However, other equivalent conventional procedures can, of course, also be used. Such conventional techniques and descriptions can be found in standard laboratory manuals such as The AGT Cytogenetics Laboratory Manual—Arsham, Marilyn S., Margaret J. Barch, and Helen J. Lawce. The AGT Cytogenetics Laboratory Manual. 4th ed. Hoboken, New Jersey: Wiley-Blackwell, 2017, all of which are herein incorporated in their entirety by reference for all purposes.

In traditional cytogenetic analysis, chromosomes are deposited on a plain glass slide while maintaining cellular registration as defined herein. In the preferred embodiments of this invention, cellular registration is lost and neighboring chromosomes might have come from the same or different cells. This generally is an undesirable loss of information, but enables the deposition of chromosomes at high spatial densities, which is beneficial for throughput.

Consistent with the above, disclosed herein are sample preparation and interrogation systems that rapidly analyze a very large number of chromosomes from one or more samples. The system comprises cellular and biochemical enrichment techniques for preparation of chromosomes from actively dividing cells, chemical compositions to encourage particle like behavior of individual chromosomes, substrates typically comprised of open fluidic devices, an apparatus for controlled deposition of chromosomes onto the substrate, staining chemistries and fluidic devices for staining in a rapid and controlled manner, an analysis instrument to interrogate the chromosomes on the device, and a computer system and software for analyzing the sum of the acquired data. The invention can be realized in a practical form as a subset of the former parts, a non-limiting example being that manual staining can be performed instead of automated staining, at the expense of requiring more human hands-on manipulations.

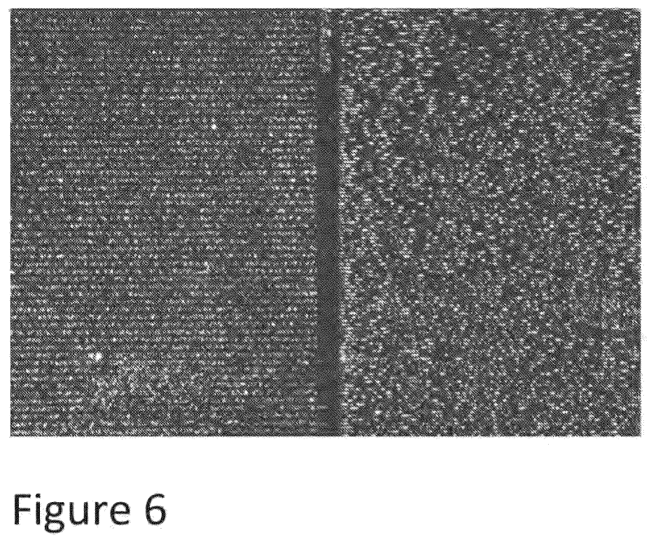

Also disclosed herein are methods to analyze genomic information with very high throughput. Extrapolating chromosome packing densities achieved in FIG. 6 to fill an 18×44 mm area—which represents the typical working area of a standard microscopy glass slide—it is possible to analyze over 46 million chromosomes. This is equivalent to the contents of one million healthy human cells, and 12 Pbp (peta basepairs, 10^15 bp) of DNA, representing 6 PBp of unique content.

Also disclosed herein are methods to search for exceedingly rare genomic events, such as looking for one aberrant cell in 100,000. Cancer cells frequently exhibit aneuploidies and lesions, but current cytogenetic techniques can only detect them when cancer cells are present in high abundance. Screening for early-stage cancer is a needle in a haystack problem in that the density of transformed cells is initially quite low. The invention can thus find cancer earlier, when therapeutic intervention has the best chance of working.

Also disclosed herein are methods to extend traditional cytogenetic analysis to look for numerical or structural abnormalities with a very large number of chromosomes, such as resulting from a heterogeneous population of cells such as a cancerous tissue. By counting occurrences of chromosomal abnormalities, it is possible to construct atlases of cancerous cells.

Also disclosed herein are methods to assess whether multiple genomic entities are present on the same or different chromosomes. When the sample is taken from a population of individuals, such as from an environmental sample of microorganisms or plants, it is possible to look for correlations or anticorrelations of multiple genes within an individual, as long as they are on the same chromosome.

Also disclosed herein are methods to generate high quality physical maps of chromosomes by controlling chromosomal positioning, including orientation, pose and relative spacing between adjacent chromosomes, prior to interrogation. Current analysis algorithms must contend with curved and bent chromosomes, which when present in a dense puddle of neighboring chromosomes, can be difficult to sort out. In particular, chromosomes that lie together can be extremely difficult to distinguish from translocations. Not only does the improved pose and straight orientation of chromosomes on the substrate improve the data quality of individual chromosomes, but the statistical sampling resulting from a massive number of chromosomes can provide a much more statistically sound diagnosis than current cytogenetic methods.

The disclosure is further understood in light of the following overview discussion of particular components, which may constituent components of systems or be used pursuant to practice of methods disclosed herein. The components are discussed preliminarily as a group, and then each section will be presented in more extensive detail later in the disclosure.

Surfaces. A number of surfaces are consistent with the disclosure herein. Any surface capable of receiving a suitable sample may be used in the simpler embodiments of the invention. For example, a glass slide onto which prepared samples may be deposited and dried, and subsequently imaged, represents one class of suitable surfaces. Most preferably, the surface comprises the surface of a substrate, or the surface of a fluidic device.

Some surfaces further comprise patterned or etched surface features, such as those that may facilitate positioning of chromosomes or nucleic acid molecules, bound or unbound, on the surface. Some such features comprise channels in the surface, such as those configured to accommodate a linear series of target molecules such as chromosomes, ecDNA molecules, or nucleic acid molecules either bound or unbound by chromosome constituents. Exemplary channels have a width comparable to target molecule widths, such that target molecules may lay down in single file along their long axes in the features but may not overlap or lay side by side.

Alternate surface features comprise inscribed or indented wells, configured in both a long and a short dimension to accommodate a single target molecule, or only molecules of a single target molecule size or smaller. For example, surface features may be configured to accommodate wild type or known structural variants of human chromosomes 1, 2, 3, 4, 5, 6, 7, 8, 9, 10, 11, 12, 13, 14, 15, 16, 17, 18, 19, 20, 21, 22, X, Y, mitochondrial DNA, or ecDNA.

Alternately or in combination, surfaces may be decorated by extruding features. Such features may serve to align or guide deposition of target molecules onto the surface. Such features may be configured to ensure or encourage separation of target molecules or other molecules deposited on the surface, such that the molecules do not overlap, are less likely to overlap, or overlap at a frequency of no more than, for example, 1% or at a frequency disclosed elsewhere herein.

Many surfaces compatible with the disclosure herein share the feature that they may receive target molecules suspended in a solution such as an aqueous solution, and hold the molecules in place noncovalently upon removal of the solvent. Often, this is accomplished without deposition of oligonucleotide probes or binder on the surface, such that the target molecules are deposited on the surface without being covalently bound to the surface or base-paired to oligos tethered to the surface. Similarly, the surfaces are in some cases not coated with or do not comprise a surface layer that specifically, covalently or noncovalently binds to deposited target molecules such as chromosomes or nucleic acid molecules, alone or bound to chromatin constituents. Alternately, some surfaces comprise a nonspecific target molecule binding moiety.

Surfaces are preferably of a size to accommodate a large number of target molecules, such as at least 1,000,000 human chromosomes, at a density such that no more than 10% of the chromosomes overlap. Some sample configurations exhibit an overlap of substantially below 1%, such as 0.5%. 0.2%. 0.1%, 0.05%, 0.02%, 0.01%, 0.005%, 0.002%, 0.001%, or less. These rates of overlap are attained on surfaces having densities of, for example, at least one per 500 micros square. Similarly, surfaces onto which nucleic acids are deposited may comprise at least 10,000 non-overlapping nucleic acid molecules positioned on the surface, and may exhibit a density of the non-overlapping nucleic acid molecules are positioned on a portion of the surface of no greater than 1000 mm square. The nucleic acid molecules comprise basepairs, and may a density of nucleic acid basepairs of at least 100 Gb/mm2 on the surface, or 200, 500, 1,000, 2,000, 5,000. 10,000 or greater than 10,000 Gb/mm2 on the surface. The at least 10,000 non-overlapping nucleic acid molecules in some cases have a median length of at least 100 kb, or 200, 500, 1,000, 2,000, 5,000, 10,000, or greater than 10,000 kb. The nucleic acids are in some cases unmodified, or synthesized in vivo, or naturally occurring, and may be chromosomes or portions of chromosomes or molecules comprising sat least some chromatin constituent molecules, such as histones H2A, H2B, H3, H4, linking histones H1, condensing 1, condensing 2, or other chromatin constituents, and may exhibit these proteins at a frequency of at least or no more than 25%, 50%, 75%, 80%, 85%, 90%, 95%, 99%, or may exhibit 100% of the chromatin constituents found in chromosomes, such as condensed chromatins in vivo.

A key feature of the surfaces herein is their ability, independent of surface coating, to tolerate sample deposition and removal of sample solvent, such as that disclosed below.

Surfaces are of a composition or possess surface characteristics conducive to imaging. That is, the surfaces possess characteristics that do not interfere with target molecule imaging. If imaging is to be performed through the surface, then the surface is to be transparent.

Surfaces are in some cases compatible with or integrated into fluidic systems, such as fluidic systems for the delivery of prepared samples for deposition onto the surface. Some such surfaces have integrated therein an outlet or outlets for the receipt of liquid reagents being delivered to the surface.

Surfaces are in some cases integrated into imaging systems, such as imaging systems having a camera or other image capture apparatus, and optionally a data storage or data transmission, or both a storage and a transmission apparatus. Alternately, some surfaces are compatible with exogenously provided imaging, data collection or data transmission apparatuses.

Imaging systems allow rapid, automated interrogation of target molecules on a surface.

Positioning. A number of sample positioning or deposition approaches are consistent with the disclosure herein. Prepared samples may be positioned on a surface directly or delivered through a microfluidic system.

Exemplary approaches comprise use of receding meniscus target molecule deposition. That is, a sample is deposited on a surface, and a receding meniscus approach is used to remove the solvent while leaving the chromosomes or other target molecules deposited on the surface. Blade coating and combing, to mention but two approaches, are types of receding meniscus approaches used in sample deposition and target molecule deposition upon solvent removal. Alternate approaches comprising, for example, evaporation of the solvent, wicking, or other approaches are also consistent with the disclosure herein. Discussion of sample deposition approaches in the art are found, for example, in U.S. Pat. No. 6,147,198, published Nov. 14, 2000, which is hereby incorporated by reference in its entirety, and U.S. Pat. No. 6,607,888, published Aug. 19, 2003, which is hereby incorporated by reference in its entirety. Additional target molecule deposition or positioning approaches include, for example, dip-coating, spinning or centripetal force, dispensing, fluid flow such as capillary flow, or direct positioning via stamping onto the surface.

A benefit of some deposition approaches herein is that they result in target molecules being oriented in parallel relative to one another relative to their long axes. This feature, alone or in combination with some of the surface features discussed above, facilitates deposition of target molecules at a high density, such as at least one per 500 microns square, while maintaining an overlap rate of no more than 1%.

Sample prep. Achieving the low rates of target molecule overlap, high densities and high overall molecule deposition amounts is enabled or facilitated in some cases through sample preparation that enriches for condensed, readily observable chromosomes in a sample. Various steps of the sample preparation disclosed herein, alone or in combination, facilitate such enrichment.

Sample preparation compositions variously comprise intact chromosomes unbound by nuclei. These are obtained by lysing cells so as to release cytosolic components without lysing nuclei, followed by differential removal of intact nuclei so as to yield condensed chromosomes from cells lacking nuclei, such as metaphase cells.

Cells may be enriched for condensed chromatin metaphase cell cycle status by, for example, culturing the cells so as to facilitate cell cycle progression, followed by or concurrently with inducing cell cycle arrest in metaphase. This can be effected by providing the cells with a cell cycle progression inhibitor such as a colcamid, or for example resveratrol or lambersulfat, or other compound that causes cell cycle arrest at metaphase or that causes chromatin condensation. Alternately or in combination, cell cycle arrest may be effected through selective nutrient withdrawal, environmental shock or other approach known in the art. As yet another alternative consistent with the disclosure herein, cells are treated with a cell cycle progression inhibitor or chromatin condensation inducing drug or treatment without culturing, or are analyzed without first enriching for metaphase cell status.

Metaphase is notable for the accumulation of condensed chromosomes and dissolution of the nuclear membrane. Thus, by lysing cell membranes while preserving nuclei, and then discarding the nuclei from the lysate, one enriches for condensed, metaphase chromosomes.

Sample preparation compositions often beneficially comprise a detergent such as a chromosome coating detergent. The components are suspended or dissolved in a solvent such as a volatile solvent, so as to facilitate target molecule deposition on the surface. The solutions are often give an acidic pH, such as no greater than or about 4, 3.5, 3, 2.5, 2, or less than 2. Exemplary compositions exhibit a pH of 2.

Compositions comprising target molecules in some cases comprise chromosomes or nucleic acids molecules harboring binding moieties such as exogenous binding moieties or native chromatin constituents such as nucleosomes, Histones H2A, H2B, H3, H4, or the nucleosome linker H1, condensin 1 and 2, or other chromatin constituents. The chromatin constituents are often present at a concentration on the nucleic acid core of at least 50%, 60%, 70%, 80%, 90%, 95%, 99%, or even 100% of the concentration on the nucleic acid core seen in native chromatin in cells.

Consistent with the compositions and methods above, disclosed herein are methods of preparing samples for cytological analysis comprising enriching for condensed chromosomes, such as my enriching for metaphase cells in a cell population, lysing the cell membranes of the cells, and discarding intact nuclei from the lysate.

Samples on Surfaces, Assaying-determinations and Rates. Application of samples as prepared herein to surfaces herein results in target molecules such as intact or partially intact chromosomes, or ecDNA or other molecules of interest, on a surface at high numbers, high density and low rates of overlap. The densities, total numbers of target molecules, and rates of overlap compare favorably to non-automated methods as currently used in the field of chromosome analysis and are conducive to automated scoring and analysis.

Assaying is readily effected through interrogation of the sample on the surface, such as through image capture. The subsequent data is conducive to automated analysis, and comprises enough nonoverlapping target molecules to render such analysis an efficient way to scale up analysis or perform high throughput analysis of large numbers of target molecules. The numbers of target molecules analyzed facilitates analysis approaches that are not available to traditional chromosome spreads, where one or a few cells contents are deposited on a surface, and contents from separate cells are not intermingled.

For example, because overlap events occur at relatively low frequency, one may discard overlaps rather than consuming substantial user time in making subjective estimates as to the chromosomes they represent.

Furthermore, because of the large number of cell contents analyzed, one may confidently detect rare events or rarely occurring chromosome or nucleic acid molecules in a sample, or conclude with a higher degree of confidence that they are not present in an individual from which a sample is taken. Similarly, one can determine with a high degree of statistical confidence the abundance of a particular chromosomal abnormality, transgenic event or contaminant in a sample population. Furthermore, one can determine the relative abundance of one target molecule population relative to a second, such as chromosomes harboring a genetic lesion relative to wild-type chromosomes, or aberrations in chromosome number as seen in ancuploidy events, even when they occur in only a subset of cells in a sample.

Assessments are made in some cases by sorting and counting types of target molecules in a given image or sample. That is, similar or identical instances of a particular first target molecule in a sample or image are grouped into a first set, while similar or identical instances of a particular second target molecule in a sample or image are grouped into a second set. Grouping may be effected on the basis of chromosomal banding patterns, predicted sequence, or presence of a label or probe. Grouping may based upon a threshold level of identity of a portion or all of a set of chromosomes as discussed elsewhere herein.

Upon assembling the first set and second set, one may, for example, compare their relative numbers so as to determine the relative abundance of, say, wild type chromosomes as compared to chromosomes of a common origin harboring a specific lesion, or of one or both relative to the abundance of a second type of lesion on the same chromosome of origin.

In particular, these comparisons or other assessments may be made with a degree of statistical confidence not attained though current methods. Accuracy of a determination is, in some cases, a function of the number of nonoverlapping target molecules in a sample. That is, if a particular chromosomal lesion is not seen in a sample comprising at least 1,000,000 target molecules, of which no more than 1% show overlap, one can conclude that the chromosomal lesion is present at a frequency of less than about 1 in one million in the assayed sample. Similarly, if a lesion is found in 100 copies in the same sample image, one may conclude that its concentration in the sample is about 1 in ten thousand. These assessments are made without reliance upon target molecule determination by human analysis of the image, though in some cases assessments are verified in full or in part by a human specialist subsequent to the analysis.

Of equal value, these assessments may be made in an automated, high throughput fashion. That is, not only are individual samples assessed with a high degree of statistical accuracy, but large numbers of samples may be assessed rapidly, without reliance on a human user to make chromosomal assessments.

Various steps or materials of the disclosure are now presented in further detail.

### Sample Preperation

Enrichment of specific cell types can occur prior to or in concert with sample preparation. In some embodiments, potentially cancerous cells can be enriched with circulating tumor or fetal cell enrichment kits or techniques such as those relying on different mechanical properties. In other embodiments, cell enrichment can take place on an integrated fluidic device that prepares cells for analysis.

In some embodiments, a fluidic device for sample preparation can be utilized for the short term culture of cells and subsequent chromosome isolation.

A wide variety of cells are amenable to analysis with the present disclosure. Immortalized cell lines have been primarily used for development, including K562 (ATCC® CCL-243), Jurkat E6-1 (ATCC® TIB-152), and A549 (ATCC® CCL-185). The invention can be applied to any type of live cells, including primary cell samples, that are routinely used for cytogenetic analysis, preferably though not exclusively cells that can be cultured for at least one cell cycle division.

For cells that are able to progress through the cell cycle, chromosomes can be arrested in a condensed state by the addition of cell-cycle synchronization agents, such as Colcemid at a suitable concentration such as 100 ng/ml. The invention is not limited to samples prepared with Colcemid, and will work with the variety of methods known in the art that synchronize cells in metaphase, such as present in Arsham, Marilyn S., Margaret J. Barch, and Helen J. Lawce. The AGT Cytogenetics Laboratory Manual. 4th ed. Hoboken, New Jersey: Wiley-Blackwell, 2017. For cells that are damaged, such as by ionizing radiation, and resist entry to mitosis, chemical additives such as Calyculin A and Okadaic acid can be used to produce premature chromosome condensation (Gotoh, Eisuke. “G2 Premature Chromosome Condensation/Chromosome Aberration Assay: Drug-Induced Premature Chromosome Condensation (PCC) Protocols and Cytogenetic Approaches in Mitotic Chromosome and Interphase Chromatin for Radiation Biology.” In Radiation Cytogenetics, edited by Takamitsu A. Kato and Paul F. Wilson, 1984: 47-60. Methods in Molecular Biology. New York, NY: Springer New York, 2019).

It is well known that the fraction of cells that possess condensed chromosomes, referred to as the Mitotic Index, can vary by cell type and chromosome condensation method. There is often observed an inverse correlation between chromosome length and mitotic index, with samples that are left in the condensed state for longer exhibiting a greater degree of axial compaction. More compact chromosomes have a mixture of desirable and undesirable properties. Dense chromosomes better resist cell lysis, in particular mechanical shearing. They are less likely to tangle upon each other in solution, when chromosomes are contacting each other in a pellet from centrifugation, during filtration and during deposition onto a substrate, be it plain glass or chemically patterned or topologically patterned. However the dense compaction limits the resolution with which cytogeneticists can distinguish genomic features, the limit often being set by the optical diffraction limit for commonly available oil immersion high NA objectives utilized with visible light. The discussion of chromosome length that follows is not completely understood in the art and is presented here without being bound by theory. For existing cytogentic methods, the aspect ratio of chromosomes is generally thought to be fixed upon cell harvest, with the width of chromosomes determined by the degree of compaction of the Condensin I complex, while axial compaction is determined by the extent of Condensin II loop extrusion in concert with Topoisomerase II and non-essential assistance from histones. A variety of “Anti-contraction agents” are known in the art, which when added during cell growth can presumably slow the rate of axial compaction via loop extrusion (Arsham, Marilyn S., Margaret J. Barch, and Helen J. Lawce. The AGT Cytogenetics Laboratory Manual. 4th ed. Hoboken, New Jersey: Wiley-Blackwell, 2017.). In some embodiments, cells are grown for the purposes of extracting chromosomes and media include nucleotide and nucleoside analogues, preferably BrdU and EdU, intercalating and DNA binding agents, preferably Actinomycin D and Ethidium Bromide. Chromosome length is often referred to in relation to standard protocols which use Colcemid to arrest metaphase cells, and prolonged exposure to Colcemid results in contracted chromosomes but achieves a higher yield of total metaphase chromosomes, so long chromosome preparation methods include cell synchronization techniques that maximize yields of metaphase chromosomes while requiring less time exposed to Colcemid. In some embodiments, cell synchronization techniques include Thymidine block, double Thymidine block, nocodazole, and preferably methotrexate. In further embodiments, phosphatase inhibitors Calyculin A and Okadaic acid are used with Colcemid, with low concentrations or short exposure times to Colcemid or in the absence of Colcemid, and are optionally used in concert with other methods to improve yields and consistency by cell cycle synchronization, preferably Thymidine block followed by short Calyculin A exposure. When the experimentalist has the luxury to choose between different cell types or tissues to sample from, it is frequently the case that longer chromosomes can be had from one type vs. another. Chromosomes can be expanded isotropically with the modulation of the ionic strength and pH of the containing solution (Beel, Andrew J., Pierre-Jean Matteï, and Roger D. Kornberg. “Mitotic Chromosome Condensation Driven by a Volume Phase Transition.” Preprint. Biophysics, Jul. 30, 2021). The application of proteases is known to isotropically expand chromosomes, and is employed to sensitize chromosomes to staining procedures such as Giemsa banding with Trypsinization, but in the present state of the art, isotropic expansion creates ghost like chromosomes that do not possess intrinsically better resolution.

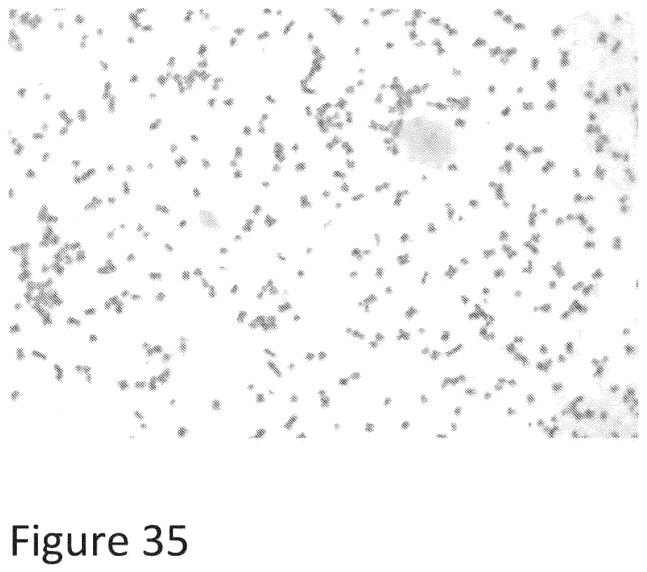

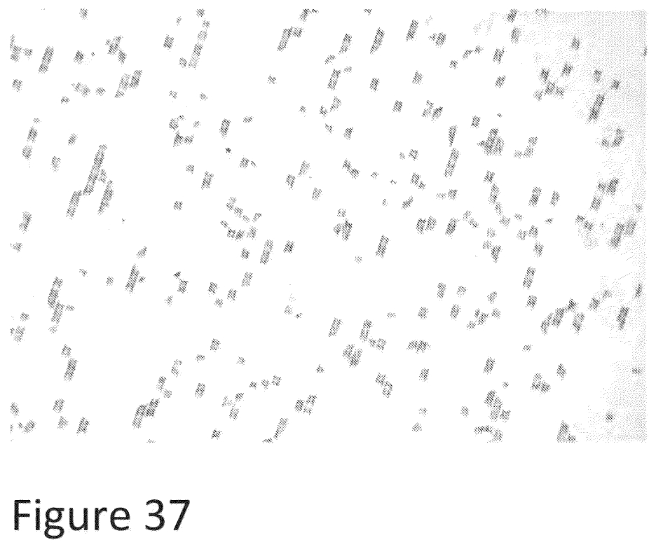

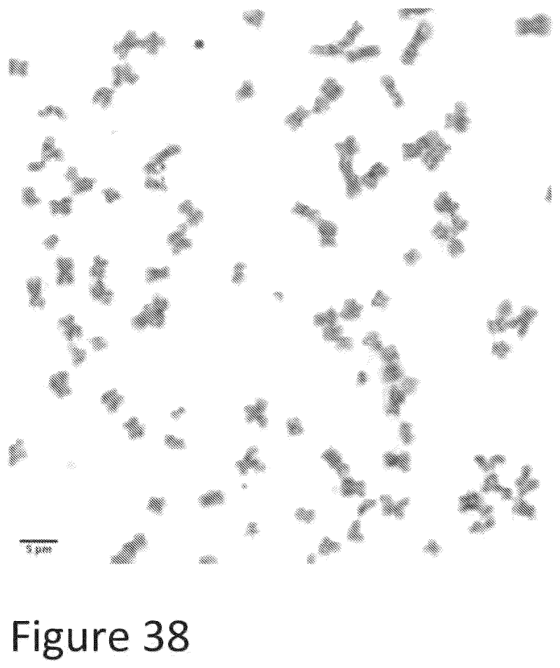

The present invention provides an opportunity to manipulate chromosomes after they have been deposited. When chromosomes are confined to a substrate with channels, the channels preserve the axial width of the chromosomes while chemical agents, biochemical agents and physical forces act to expand chromosomes in a manner that would in the current state of the art, expand chromosomes isotropically and not gain the increase in axial ratio necessary to obtain high resolution information. Such methods of elongation are not limited to changing the composition and ionic strength of salts, preferably reducing ionic strength and depleting di-valent and multi-valent cations, addition of proteases to digest chromatin, addition of phosphatases, acetylases and methyl transferases to alter the constituents of chromatin, addition of agents that modulate the persistence length of DNA, preferably increasing it, agents that disrupt protein-nucleic interactions, co-solvents or chaotropic agents such as formamide that disrupt nucleic acid and protein structures, fluidic flow, electrophoretic forces, magnetic forces in concert with magnetic handles on the chromosomes, most preferably iron nanoparticles, and centrifugal forces. A demonstration is shown in FIG. 37, wherein chromosomes were subjected to a long duration of trypsin digestion (180 seconds) and elongated, in comparison with the same sample deposited on a plain glass substrate and digested for only 45 s in FIGS. 35 and 38.

Cell lysis may accomplished by a variety of approaches such as incubation with detergents such as digitonin, saponin, NP-40, Triton X-100, subjecting cells to mechanical forces such as vortexing, dounce homogenization, sonication, passage through fine needles and repeated freeze thawing. The cell lysis buffer can contain chemical additives to preserve chromosome morphology, including protease and nuclease inhibitors, crowding agents such as sucrose, and mono-, di-, and multi-valent cations such as potassium, magnesium and spermidine. The sample pH can vary from highly acidic to neutral, preferably pH 2.0-7.5. In preferred embodiments, cells are lysed using a nuclear isolation process that is gentle enough to rupture cell walls, but not harsh enough to rupture intact nuclei; since the nuclear envelope has already broken down in mitotic cells, chromosomes are liberated by the nuclear isolation process while interphase DNA is held within nuclei. In some embodiments, the mixture of nuclei and chromosomes is separated by methods that exploit the physical size difference between nuclei and chromosomes as described below.

In some embodiments, cells are treated before, during or soon after lysis with agents that modify chromosomes and chromatin, such as heating, cooling, sonic disruption, microwave radiation, ultra-violet radiation, bead beating, chemical agents including ions, chemical agents including fixatives and chemical agents that modify RNA and chromatin. For modification before lysis, cell permeabilization agents can be used, preferably digitonin, saponin, saponins, and low concentrations of non-ionic detergents. A variety of ions can be employed as chemical agents, such as described in classical literature on chromosomal preparations Sone, Takefumi, Megumi Iwano, Shouhei Kobayashi, Takeshi Ishihara, Naoto Hori, Hideaki Takata, Tatsuo Ushiki, Susumu Uchiyama, and Kiichi Fukui. “Changes in Chromosomal Surface Structure by Different Isolation Conditions.” Archives of Histology and Cytology 65, no. 5 (2002): 445-55, such as acetate, ammonium, cesium, chloride, cobalt, citrate, hydride, hydroxyl, magnesium, manganate, phosphate, potassium, spermine, spermidine, triethylamine, and zinc. A variety of fixation agents can be employed, not limited to Alcoholic Formaline Fixative, formaldehye, paraformaldehyde, Ammonium Sulfate Fixative, BOUIN Fixing Solution, BOUIN-HOLLANDE's Fixative for IHC, BOUINs reagent (4% formaldehyde), Bouin Allen Fixative, CARNOY Fixing Solution (Chloroform), CARNOY Fixing Solution (Methanol and Acetic Acid), CARNOY fixing solution (formaldehyde-alcohol-acetic acid), CARNOY's Fixative (Chloroform & Iron(III) Chloride), Chromic Acetic Acid Fixative, Chromic Acid 2-15%, DAVIDSON solution, DELAUNAY's Fixative, Diethyl Ether Faxiation, ESPOSTI's Fixative for Urine Cytology, Ethanol Glacial Acetic Acid Fixative, FREIBURG's Fixation, Fixative after THIEL, Fixing solution according to STIEVE, Formaldehyde fixing solution for Bulbi, Formalin 2-37% whether buffered, unbuffered, acid free, with eosin, Formalin-acetone, Fixative F13, Zinc Fixative, Glutaraldehyde 0.65-6.5% with or without buffer, Glutaraldehyde fixing solution according to KARNOVSKY, Glutraldehyde Formaldehyde Cacodylate, JORES fixing solution, KAISERLING's Fixative, Merthiolate Formaline Solution, NAWASHIN's Fixative, Bi-functional imido-esters, Bi-functional NHS-esters including disuccinimidyl glutarate, O,O′-Bis[2-(N-Succinimidyl-succinylamino)ethyl]polyethylene glycol and dithiobis[succinimidyl propionate], Bi-functional maleimides, heterobifunctional crosslinkers including imido-ester NHS-ester crosslinkers, Paraformaldehyde (PFA) 4-10%, whether unbuffered or buffered, Paraformaldehyde (PFA) 4%, in Glutaraldehyde 0.5% & PBS pH 7.4, Picnic Acid, ROSSMANN's Fixative, SACCOMANNO's Fixative, SCHAFFER's Fixative, SCHAUDINN's Fixative, Sodium Carbonate Formaline after KOSSA, Trichloroacetic Acid Mercury Formol after ROMEIS, Wintergreen Oil after SPALTEHOLZ, ZAMBONI solution, ZENKER's Fixative, Zinc Chloride—Acetic Acid—Formaline. Agents that modify RNA and chromatin are not limited to RNAse, preferably RNAse A, polyamines, EDTA, proteases, preferably pepsin, Polyvinylpyrrolidone, coblock polymers, coblock polymers incorporating positively charged regions, most preferably PEI-g-PEG, small RNA preferably yeast RNA-phe, bulk proteins, preferably BSA, organic acids preferably acetic acid, and mineral acids, preferably dilute HCl.

Cells may be treated before or during lysis, or their lysates may be treated soon after lysis with agents that prevent modification of chromosomes and chromatin, such as inhibitors of nucleases and proteases, preferably PMSF, and anti-oxidizing agents preferably dithiothreitol.

It is often desirable to stabilize chromosomes and prevent one chromosome from becoming entangled with another chromosome. In some embodiments, chromosomes are coated with or encapsulated within a layer of one or more of the following: dextran, starch, hyaluronic acid, chitosan, protein aggregates including protein aggregates formed from collagen, gelatin and albumin, poly(lactide-co-glycolide), (3-hydroxybutyrate-co-3-hydroxyvalerate), poly(sebacic anhydride) and poly(s-caprolactone), poly(N-isopropyacrylamide), Eudragit L100 and S100, and high molecular weight polymers including homopolymers, co-block polymers and dentritic polymers, the polymers comprised of various moieties including but not limited to polyethylene oxide, polyethyleneimine, polyacrylamide, polyN-isopropylacrylamide, and polyamide. Some polymers contain cross-linking agents, and or heterofunctional molecules that possess one moiety that binds to chromatin and another moiety that binds to or participates in a covalent bond with the polymeric matrix, and in all cases the cross linkers can optional possess an additional moiety that permits selective cleavage, preferably a disulphide linkage. The particles can later be degraded by the addition of chemical moieties that do not also degrade chromosomes, including alginate degradation by chelation or pH change, Eudragit degradation by pH modification, and polymeric bead degradation by enzymatic digestion.

Centrifugation can be used to separate chromosomes from faster sedimenting contaminants such as cell lysis debris, intact and ruptured nuclei, and also from more slowly sedimenting contaminants such as uncondensed interphase chromatin, mRNA, ribosomes and other substantial contaminants. Centrifugation can take place in buffers with density and viscosity similar to phosphate buffered saline, or solutions with greater density or viscosity such as sucrose, percoll, ficoll or cesium chloride.

Alternately or in combination, samples can be enriched for chromosomes by means of filtration. Crude filter pores permit chromosomes to pass but block large debris, and fine pores can be used to collect chromosomes and pass through lighter contaminants such as interphase DNA.

Alternately or in combination, samples can be enriched by the application of fluidic shear forces, such as experienced when pipetting vigorously and repeatedly, that are strong enough to break individual strands of un-condensed DNA but are not strong enough to cause significant damage to chromosomes. In addition to pipetting, it is possible to create such fluidic forces by passage through a narrow orifice such as a 25 ga needle, between two plates or within a fluidic structure embedded in a microfluidic device. Flowing a mixture of chromosomes and contaminating interphase DNA across a network of fine pillars will also result in a combination of filtration and breakage. Additional purification can be obtained by gentle and infrequent cleavage of nucleic acids and or proteins attached to nucleic acids that contribute to chromosomal stability, at a rate of cleavage that leaves chromosomes substantially intact but which cleaves uncondensed nucleic acids into fragments, preferably between 10 and 50 kbp.

In some embodiments, a desired set of chromosomes or chromatin material may be enriched prior to application to the fluidic device for analysis. For example, the set may comprise enriched ecDNA, or enriched X chromosomes, or enriched Y chromosomes, or enriched chromosomes of human type 1, or enriched chromosomes of types 2, 3, and 4.

The chromosomes may originate from cells that have been pre-selected based on a criteria such as morphology, or the presence of a labeling body, or the lack of a labeling body, or the type of cell, or the stage of the chromosome condensation within the cell.

In some embodiments, cells are barcoded prior to lysis with barcodes that bind covalently or non-covalently to nucleic acids, chromatin or chromosomes and stay with that material during deposition onto a fluidic surface and interrogation. Some barcoding methods include barcoding methods described herein, most preferably using SPAAC chemistry to add nucleic acid barcodes to genomic material that has incorporated the metabolic label 5-(Azidomethyl)-2′-deoxyuridine (AmdU) and the nucleic acid barcodes comprise a DNA strand with a bicyclo[6.1.0]nonyne (BCN) moiety. In some embodiments, cells are isolated into containers, wells or droplets that hold one or more cells, and combined with barcodes. In exemplary embodiments, cells are hypotonically swollen, permeabilized with a mixture containing dilute saponins and a fixative prior to isolation and barcoding.

### Chromosome Condensation Densities

Chromosomes can vary in the degree of condensation, specifically measured as linear density which is the amount of DNA per unit length of the chromosome. In an uncondensed, fully elongated form, B-form DNA has a linear density of 0.003 Mb/um. Upon chromosomal condensation, this can increase 4 orders of magnitude, with 40 Mbp/um routinely observed in our hands when chromosomes are both well condensed at the time of cell harvest, and then prepared in a chemical buffer which favors condensation. At the other extreme, 5-7 Mbp/um is possible but difficult when viewing highly elongated prometaphase chromosomes, Yunis, Jorge J. “Mid-Prophase Human Chromosomes. The Attainment of 2000 Bands.” Human Genetics 56, no. 3 (February 1981): 293-98, Drouin R, Lemieux N, Richer C L. High-resolution R-banding at the 1250-band level. III. Comparative analysis of morphologic and dynamic R-band patterns (RHG and RBG). Hereditas. 1991; 114(1): 65-77.

Chromosomes and chromatin packing densities are influenced by multiple factors, At a physical level, chromatin behaves as axially scaffolded ionic hydrogel (Beel 2021). Once removed from a cell and free from interference from proteases and phosphatases, chromosomes can reversibly expand and contract as a function of ionic strength, ionic multivalency, and water activity. The form of the condensed chromosome, in particular its length to width ratio, is determined by a complex interplay of biological factors. Nucleosomes, Condensin I and II and Topoisomerase II all play significant roles in the establishment of the chromosome axial scaffold and chromosome condensation.

There are several different impacts of condensation. From the perspective of imaging resolution, less linear density results in an increase in genomic resolution. Basic brightfield or fluorescent imaging is limited to a resolution at the Rayleigh limit of about 220 nm with 500 nm light and a 1.4 NA microscope objective and the sample embedded in a high refractive index medium. With a packing density of 40 Mbp/um, this corresponds to a genomic resolution of 8 Mbp. At a packing density of 5 Mbp/um, this corresponds to a genomic resolution of about 1 Mbp.

### Total Amount of Target Molecules Per Device

The approaches herein scale up easily with regard to the total number of target molecules such as chromosomes that can be analyzed. Cell growth, mitotic index and the yield of harvested chromosomes may determine how many target molecules such as chromosomes are readily prepared per sample. The final density of chromosomes on the substrate, the space between chromosomes along a topological feature or patterned chemical feature, space between adjacent parallel topological or chemical features, and the total substrate area combine together to influence the total number of chromosomes that can be detected per substrate. During interrogation, the scanning speed of an interrogation moiety and spatial resolution place practical constraints on the time need to interrogate the whole sample, store the raw physical data and perform algorithmic or human-read extraction of chromosomal features.

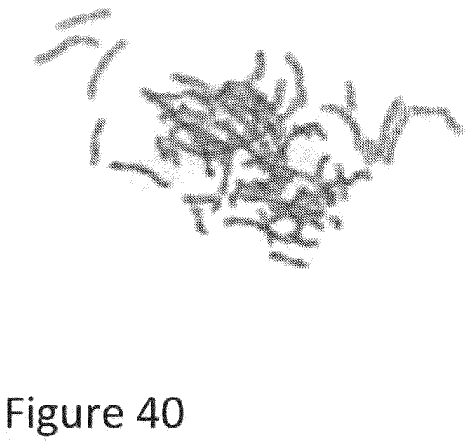

As a comparison, cytogenetic protocols for preparing chromosome spreads on a glass slide (25 mm×75 mm) for imaging typically yield about a maximum of 700 metaphase cells per slide, and often with substantially more than 1% overlap. See, for example, FIG. 40. This value is obtained from dispensing approximately 2-3 drops of 20-50uL of a solution, with cell concentration of 100,000 cells/mL, where the mitotic index is around 3-5%, and approximately 80% of the cells dropped on the surface adhere to the substrate. 700 metaphase cells could in theory yield 33,600 chromosomes, however practical limitations associated with the random placement of burst cells on the glass slide greatly reduces this number. Locations on the slide where the cell bursts releasing the chromosomes have a very high localized density of chromosomes randomly positioned within the cell spread, and consequently, these chromosomes have a very high probability of overlapping each other, obfuscating the ability of imaging to discern each chromosome's identity FIG. 40 demonstrates a typical cell spread of overlapping chromosomes generated by standard cytogenetic methods. As such, most cytogenetic slide protocols, Uttamatanin, Ravi, Peerapol Yuvapoositanon, Apichart Intarapanich, Saowaluck Kaewkamnerd, Ratsapan Phuksaritanon, Anunchai Assawamakin, and Sissades Tongsima. “MetaSel: A Metaphase Selection Tool Using a Gaussian-Based Classification Technique.” BMC Bioinformatics 14, no. S16 (October 2013): S13, are expected to yield only 10s of useful cell spreads, and thus only ~1000 useful chromosomes.

Through the present disclosure, however, there are in some cases at least 10,000 non-overlapping chromosomes positioned on the surface of a substrate, or at least 20,000 non-overlapping chromosomes positioned on the surface of a substrate, or at least 50,000 non-overlapping chromosomes positioned on the surface of a substrate, or at least 100,000 non-overlapping chromosomes positioned on the surface of a substrate, or at least 200,000 non-overlapping chromosomes positioned on the surface of a substrate, or at least 500,000 non-overlapping chromosomes positioned on the surface of a substrate, or at least 1,000,000 non-overlapping chromosomes positioned on the surface of a substrate, or at least 2,000,000 non-overlapping chromosomes positioned on the surface of a substrate, or at least 5,000,000 non-overlapping chromosomes positioned on the surface of a substrate, or at least, 10,000,000 non-overlapping chromosomes positioned on the surface of a substrate.

In some embodiments, at least 1,000 non-overlapping chromosomes are positioned on a 1 cm cubed area of a substrate surface, or at least 2,000 non-overlapping chromosomes are positioned on a 1 cm cubed area of a substrate surface, or at least 5,000 non-overlapping chromosomes are positioned on a 1 cm cubed area of a substrate surface, or at least 10,000 non-overlapping chromosomes are positioned on a 1 cm cubed area of a substrate surface, or at least 20,000 non-overlapping chromosomes are positioned on a 1 cm cubed area of a substrate surface, or at least 50,000 non-overlapping chromosomes are positioned on a 1 cm cubed area of a substrate surface, or at least 100,000 non-overlapping chromosomes are positioned on a 1 cm cubed area of a substrate surface, or at least 200,000 non-overlapping chromosomes are positioned on a 1 cm cubed area of a substrate surface, or at least 500,000 non-overlapping chromosomes are positioned on a 1 cm cubed area of a substrate surface, or at least, 1,000,000 non-overlapping chromosomes are positioned on a 1 cm cubed area of a substrate surface.

In some embodiments, non-overlapping chromosomes positioned on the surface of a substrate achieve an average genomic surface density content of at least 0.001 Mbp/um2 on a 1 cm cubed area of substrate surface, or non-overlapping chromosomes positioned on the surface of a substrate achieve an average genomic surface density content of at least 0.01 Mbp/um2 on a 1 cm cubed area of substrate surface, or non-overlapping chromosomes positioned on the surface of a substrate achieve an average genomic surface density content of at least 0.1 Mbp/um2 on a 1 cm cubed area of substrate surface, or non-overlapping chromosomes positioned on the surface of a substrate achieve an average genomic surface density content of at least 1 Mbp/um2 on a 1 cm cubed area of substrate surface.

In some embodiments, at least 10,000 non-overlapping chromosomes positioned on the surface of a substrate are interrogated, or at least 20,000 non-overlapping chromosomes positioned on the surface of a substrate are interrogated, or at least 50,000 non-overlapping chromosomes positioned on the surface of a substrate are interrogated, or at least 100,000 non-overlapping chromosomes positioned on the surface of a substrate are interrogated, or at least 200,000 non-overlapping chromosomes positioned on the surface of a substrate are interrogated, or at least 500,000 non-overlapping chromosomes positioned on the surface of a substrate are interrogated, or at least 1,000,000 non-overlapping chromosomes positioned on the surface of a substrate are interrogated, or at least 2,000,000 non-overlapping chromosomes positioned on the surface of a substrate are interrogated, or at least 5,000,000 non-overlapping chromosomes positioned on the surface of a substrate are interrogated, or at least 10,000,000 non-overlapping chromosomes positioned on the surface of a substrate are interrogated.

In some embodiments, at least 1,000 non-overlapping chromosomes positioned on the surface of a substrate are interrogated and at least one piece of genomic information determined for each of the chromosomes, or at least 2,000 non-overlapping chromosomes positioned on the surface of a substrate are interrogated and at least one piece of genomic information determined for each of the chromosomes, or at least 5,000 non-overlapping chromosomes positioned on the surface of a substrate are interrogated and at least one piece of genomic information determined for each of the chromosomes, or at least 10,000 non-overlapping chromosomes positioned on the surface of a substrate are interrogated and at least one piece of genomic information determined for each of the chromosomes, or at least 20,000 non-overlapping chromosomes positioned on the surface of a substrate are interrogated and at least one piece of genomic information determined for each of the chromosomes, or at least 50,000 non-overlapping chromosomes positioned on the surface of a substrate are interrogated and at least one piece of genomic information determined for each of the chromosomes, or at least 100,000 non-overlapping chromosomes positioned on the surface of a substrate are interrogated and at least one piece of genomic information determined for each of the chromosomes, or at least 200,000 non-overlapping chromosomes positioned on the surface of a substrate are interrogated and at least one piece of genomic information determined for each of the chromosomes, or at least 500,000 non-overlapping chromosomes positioned on the surface of a substrate are interrogated and at least one piece of genomic information determined for each of the chromosomes, or at least 1,000,000 non-overlapping chromosomes positioned on the surface of a substrate are interrogated and at least one piece of genomic information determined for each of the chromosomes.

In some embodiments, at least 1,000 non-overlapping chromosomes positioned on the surface of a substrate are interrogated in less than an hour, or at least 2,000 non-overlapping chromosomes positioned on the surface of a substrate are interrogated in less than an hour, or at least 5,000 non-overlapping chromosomes positioned on the surface of a substrate are interrogated in less than an hour, or at least 10,000 non-overlapping chromosomes positioned on the surface of a substrate are interrogated in less than an hour, or at least 20,000 non-overlapping chromosomes positioned on the surface of a substrate are interrogated in less than an hour, or at least 50,000 non-overlapping chromosomes positioned on the surface of a substrate are interrogated in less than an hour, or at least 100,000 non-overlapping chromosomes positioned on the surface of a substrate are interrogated in less than an hour, or at least 200,000 non-overlapping chromosomes positioned on the surface of a substrate are interrogated in less than an hour, or at least 500,000 non-overlapping chromosomes positioned on the surface of a substrate are interrogated in less than an hour, or at least 1,000,000 non-overlapping chromosomes positioned on the surface of a substrate are interrogated in less than an hour.

Accordingly, disclosed herein are systems comprising target molecules arrayed on a surface in the amounts or densities disclosed herein, as well as methods of depositing target molecules so as to attain target molecules arrayed on a surface in the amounts or densities disclosed herein.

### Deposition Buffer

The deposition buffer contains chemical additives that influence the performance of the deposition step through a variety of physical and chemical methods.

In some embodiments detergents are added to the deposition buffer, preferably non-ionic detergents, not limited to co-block polymers containing one or more polyethylene oxide chains and one or more aliphatic chains or chains with a octanol water partition coefficient log Pow>2.5, Brij 48 and derivatives, digitonin, Genapol X-080 and derivatives, IGEPAL, Kolliphor P 407 and derivatives, Methoxypolyethylene glycol 350, n-Nonyl-β-D-Glucopyranoside, Nonidet P-40 and its alternatives, Nonylphenyl-polyethyleneglycol acetate, Octaethylene glycol monododecyl ether, Pluronic F-68 and derivatives, Poly(ethylene glycol), Polysorbate 20 and derivatives, Saponin, Span 20 and derivatives, Tween 20 and derivatives, Tergitol and derivatives, Triton X-100 and derivatives and Undecyl-β-D-maltoside.

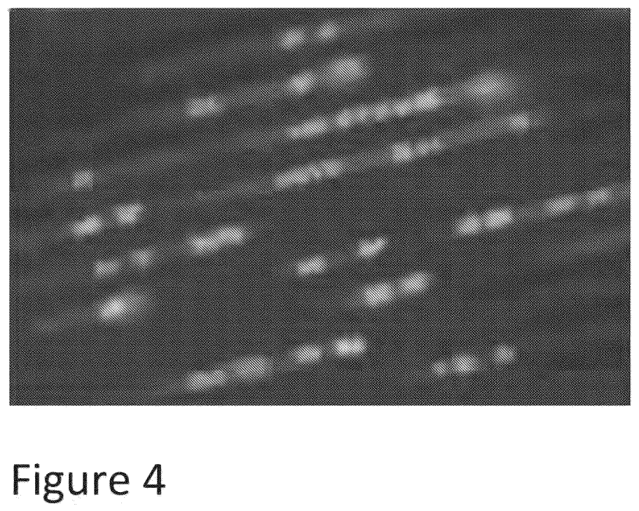

Without being bound by theory, detergents may control the surface tension of the deposition fluid, and can also change the chemical interaction energy of the chromosomes and the substrate. This is important because if the chromosomes happen to stick to the first surface that they encounter, this will result in chromosomes sticking non-specifically to aspects of the substrate topological patterns when it is desired that they only adhere to specific portions of the substrate patterns. Detergents must not be so harsh as to denature the proteins that hold chromosomes together, for example SDS in high concentrations can rapidly disrupt chromosomes and cause them to dissolve prior to surface deposition. Detergents also influence the interactions between nucleic acids, chromatin and chromosomes with the fluidic device, and can help chromosomes pose in an undistorted shape (FIG. 4) despite being positioned inside a channel that contains edges capable of capillary pull that can otherwise exert pulling forces on materials.

In some embodiments, the deposition buffer is comprised in part of ions, preferably ions that stabilize the structure of chromatin as discussed above, and most preferably ions that stabilize chromatin that do not leave a residue after the chromosomes, including but not limited to volatile ions, preferably triethylamine and acetate, volatile organic acids, ions that are effective at chromatin stabilization in low concentrations including multivalent metals, preferably magnesium, multivalent amines including spermine and spermidine, and positively charged polymers including polyethyleneimine, co-block polymers including polyethyleneimine, and zwiterionic or multivalent polymers that contain a mixture of positive and negative charges at the pH of the deposition buffer, preferably with a pl greater than the pH of the deposition buffer.

In some embodiments, the fluidic device contains surface features and surface chemical characteristics of sufficient hydrophobicity or superhydrophobicity that a drop of pure water or a physiological buffer such as Phosphate Buffered Saline will not wet the entirety of the surface but will rather position itself on the outer portions of the surface, in a Cassie-Baxter state. The deposition buffer in such embodiments can contain wetting agents to enable a greater degree of wetting of the surface features or substantially complete wetting of the surface features. In some embodiments the composition of the deposition buffer is initially sufficient to initiate greater or substantially complete wetting, but changes due to selective evaporation of the wetting agent such that the remaining deposition buffer is in a history dependent state whereby it is currently wetting features to a degree that by itself it is incapable of wetting, by virtue of it having previously contained wetting agents. One such embodiment is a surface comprised of a rectangular grating of duty cycle 50%, pitch 3 micron, depth 3 micron, fabricated in a bisphenol A based resin, preferably Su8, and the deposition buffer initially contains a mixture of methanol, acetic acid and water, preferably in the ratios 5:1:4. In further embodiments, chromosomes are placed on the device inside a buffer that by itself does not have the ability to fully wet the surface, and before the deposition buffer has fully evaporated, wetting is subsequently achieved by the addition of wetting agents to the layer of deposition buffer. In further embodiments, the wetting is achieved in a spatially selective and addressable manner by the application of a small volume of wetting agent via pipetting or microdispensing. In the cases where the amount of wetting agent is substantial enough to wet the entire surface, the regions that contact the wetting agent wet first, otherwise only a portion of the surface will fully wet, allowing controlled introduction of chromosomes to a restricted region of the fluidic surface.

In some embodiments, the deposition buffer contains additives that have a higher boiling point than water, additives including but not limited to DMSO, ethylene glycol, xylene, acetamide, n-butanol, acetic acid and formic acid. In alternate embodiments, the deposition buffer contains additives that have a lower boiling point than water, additives including but not limited to short chain alcohols including n-propanol, tert-butanol, isopropanol, ethanol and methanol, volatile acids including trifluoroacetic acid, and solvents including acetonitrile.

In some embodiments, the deposition buffer contains spacer particles, nanoparticles, microparticles or beads that are of similar size to chromosomes, preferably with one dimension between 1.2 and 1.5 um and another dimension between 0.4 and 1.2 um, and which pack inside fluidic device surface features in similar ways to chromosomes. The composition of spacer particles include but are not limited to polystyrene beads, melamine beads, quantum dots, iron nanoparticles, alginate particles, agarose beads, gelatin beads, beads comprised of hydrogels, beads comprised of acrylic moieties, silica particles, gold particles, zirconium beads, microorganisms including bacteria, dextran beads, starch beads, hyaluronic acid particles, chitosan particles, protein aggregates including protein aggregates formed from collagen, gelatin and albumin, poly(lactide-co-glycolide) particles, (3-hydroxybutyrate-co-3-hydroxyvalerate) particles, poly(sebacic anhydride) particles and poly(e-caprolactone) particles, poly(N-isopropyacrylamide) particles and Eudragit L100 Eudragit S100 particles. Most preferably, the particles can be degraded by the addition of chemical moieties that do not also degrade chromosomes, including alginate degradation by chelation, Eudragit degradation by pH modification, and polymeric bead degradation by enzymatic digestion.

### Methods of Positioning Target Molecules on a Surface

A variety of embodiment methods and devices can be used to position chromosomes or other target molecules on a surface. In some embodiments, the method may comprise a deposition method to position chromosomes suspended in a solution onto a surface. Said deposition method may comprise combing, blade coating, patterned-blade coating, bar coating, solution shearing, slot-die coating, zone casting, knife coating, wicking, flow coating, flow-cell coating, dispensing, dropping, spin coating, off-center spin coating, casting, edge casting, sandwich casting, dispensing, spraying, tape coating, dip coating, meniscus coating. In some embodiments, the device or method may comprise a substrate, a substrate comprising fluidic features, a patterned substrate, a functionalized substrate, a fluidic device, a fluidic device comprising fluidic features, or an open fluidic device. In the preferred embodiment, the result of positioning the chromosomes on a surface comprises having at least one of said chromosomes immobilized to the surface on which said at least one molecule is positioned.

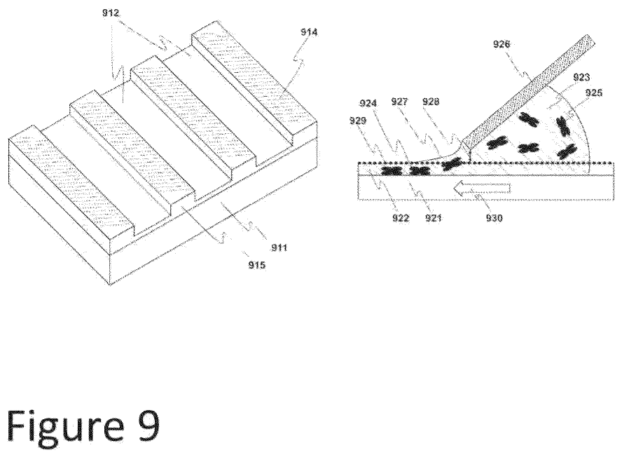

FIG. 9 demonstrates one particular embodiment of positioning chromosomes on a surface, whereby a solution of chromosomes are positioned on a surface with blade coating. In this particular drawn embodiment, the surface is the surface of an open fluidic device comprising fluidic features. The fluidic features are channels (912) and walls (915) patterned on the surface of an open fluidic device (911), whereby the top portion of the walls comprise a surface property that differs from the bottom of the channels (912). In some embodiments, the property comprises surface energy. In some embodiments, the property comprises hydrophobicity. In some embodiments, the property comprises anti-fouling. In some embodiments, the property comprises surface roughness. In the preferred embodiment, as demonstrated in FIG. 9, the top portion of the walls are more hydrophobic than the bottom of the channels, however in some embodiments, the top portion of the walls are more hydrophilic than the bottom of the channels.

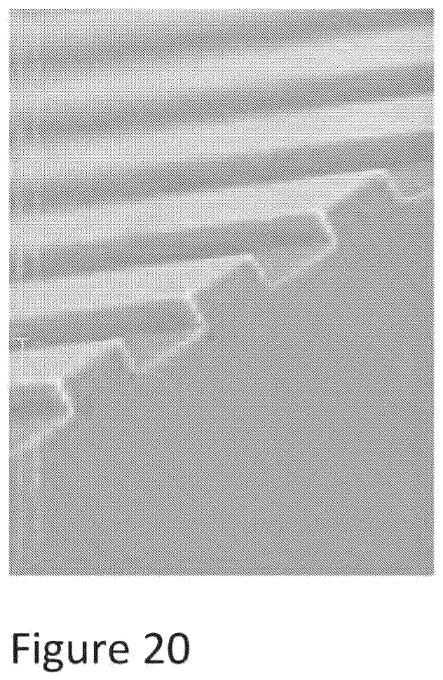

The chromosomes are positioned on the surface of the fluidic device by bringing a blade (926) within proximity to the open fluidic device (921), such that a critical distance (928) is obtained between the blade and the surface of the device such that bubble of solution (923) containing the chromosomes (925) can make contact with both the blade and the surface of the fluidic device. With the blade and device in fluidic contact, the device is then moved (930) relative to the physical position of the blade. As the relative movement of the device and blade progresses, a trailing meniscus (927) forms on the obtuse angle side of the blade, and the trailing meniscus removes the solution from the wall top (929), while leaving the channel (922) wetted with the solution and thus positioning the DNA molecule (924) in the channel. An example of such an embodiment is shown in FIG. 6, whereby chromosomes are positioned within the channels of a fluidic device. A further example of such an embodiment is shown in FIG. 20, showing a cross-sectional SEM of channels for an open fluidic device.

In some embodiments, the relative movement of the open fluidic device and the blade comprises the reverse direction, such that the trailing meniscus extends from the acuate angle side. In some embodiments, the angle at which the blade edge extends along the surface of the open fluidic device, and the relative direction of movement during operation is 90 degrees, or between 90 and 95 degrees, or between 95 and 100 degrees, or between 100 and 105 degrees, or between 105 and 110 degrees, or between 110 and 115 degrees, or between 115 and 120 degrees, or between 120 and 125 degrees, or between 125 and 130 degrees, or between 130 and 135 degrees, or between 135 and 140 degrees, or between 140 and 145 degrees, or between 145 and 150 degrees, or between 150 and 180 degrees.

In some embodiments, the bubble is positioned on the acute angle side of the blade, or the obtuse angle side of the blade, or both sides of the blade.

In some embodiments, the angle at which the blade edge extends along the surface of the open fluidic device, and the major axis of the channels is between 90 and 95 degrees, or between 95 and 100 degrees, or between 100 and 105 degrees, or between 105 and 110 degrees, or between 110 and 115 degrees, or between 115 and 120 degrees, or between 120 and 125 degrees, or between 125 and 130 degrees, or between 130 and 135 degrees, or between 135 and 140 degrees, or between 140 and 145 degrees, or between 145 and 150 degrees, or between 150 and 180 degrees.

In some embodiments, the relative motion is between 0 and 10 microns per second, or between 10 and 20 microns per second, or between 20 and 50 microns per second, or between 50 and 100 microns per second, or between 100 and 200 microns per second, or between 200 and 500 microns per second, or between 500 and 1000 microns per second, or 1000 microns per second and higher.

In some embodiments, the separation distance (928) between the surface of the open fluidic device and the blade is less than 10 microns, or less than 20 microns, or less than 30 microns, or less than 50 microns, or less than 75 microns, or less than 100 microns, or less than 200 microns, or less than 500 microns, or less than 1000 microns, or less than 2 mm, or less than 5 mm. In some embodiments, the separation distance is determined by a physical spacer that is positioned between the surface of the open fluidic device and the blade. In some embodiments, the physical spacer is integrated in the blade. In some embodiments, the physical spacer is integrated in the fluidic device.

In some embodiments, the blade comprises a fluidic device, in some embodiments, the blade comprises an open fluidic device. In some embodiments, the blade comprises fluidic features. In particular, the fluidic features may comprise open fluidic channels that improve control of the laminar flow of the solution of chromosomes onto the target surface to which said chromosomes will be positioned. In some embodiments, the blade comprises a substrate.

In some embodiments, the surface onto which the chromosomes are positioned by blade coating does not comprise patterned features.

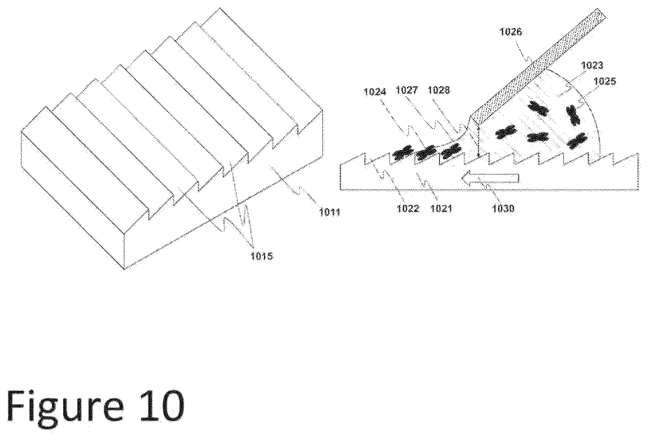

FIG. 10 demonstrates one particular embodiment of positioning chromosomes on a surface, whereby a solution of chromosomes are positioned on a surface with blade coating. In this particular drawn embodiment, the surface is the surface of an open fluidic device comprising fluidic features. The fluidic features are topological features comprising ridges (1015) patterned on the surface of an open fluidic device (1011). In this particular drawn embodiment, the surface properties of the wedges are uniform, and the topological edges of ridges provide a means of pinning the meniscus momentarily as the blade progresses across the surface of the fluidic device. In other embodiments, the ridges may comprise regions of different surface properties. In some embodiments, the property comprises surface energy. In some embodiments, the property comprises hydrophobicity. In some embodiments, the property comprises anti-fouling. In some embodiments, the property comprises surface roughness.

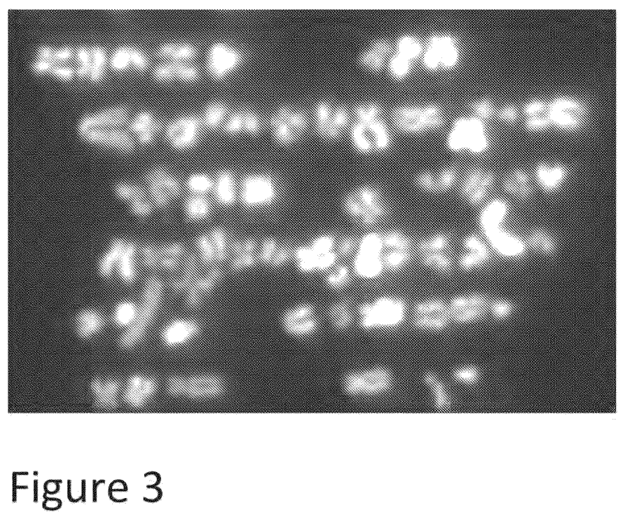

The chromosomes are positioned on the surface of the fluidic device by bringing a blade (1026) within proximity to the open fluidic device (1021), such that a critical distance (1028) is obtained between the blade and the surface of the device such that bubble of solution (1023) containing the chromosomes (1025) can make contact with both the blade and the surface of the fluidic device. With the blade and device in fluidic contact, the device is then moved (1030) relative to the physical position of the blade. As the relative movement of the device and blade progresses, a trailing meniscus (1027) forms on the obtuse angle side of the blade, and the trailing meniscus is intermittently pinned to the edges of the ridges, and positions the chromosomes (1024) along said ridges (1022). An example of such an embodiment is shown in FIG. 3, whereby chromosomes are positioned on the surface of a PDMS substrate patterned with ridges.

In some embodiments, the relative movement of the open fluidic device and the blade comprises the reverse direction, such that the trailing meniscus extends from the acuate angle side. In some embodiments, the angle at which the blade edge extends along the surface of the open fluidic device, and the relative direction of movement during operation is 90 degrees, or between 90 and 95 degrees, or between 95 and 100 degrees, or between 100 and 105 degrees, or between 105 and 110 degrees, or between 110 and 115 degrees, or between 115 and 120 degrees, or between 120 and 125 degrees, or between 125 and 130 degrees, or between 130 and 135 degrees, or between 135 and 140 degrees, or between 140 and 145 degrees, or between 145 and 150 degrees, or between 150 and 180 degrees.

In some embodiments, the bubble is positioned on the acute angle side of the blade, or the obtuse angle side of the blade, or both sides of the blade.

In some embodiments, the angle at which the blade edge extends along the surface of the open fluidic device, and the major axis of the channels is between 90 and 95 degrees, or between 95 and 100 degrees, or between 100 and 105 degrees, or between 105 and 110 degrees, or between 110 and 115 degrees, or between 115 and 120 degrees, or between 120 and 125 degrees, or between 125 and 130 degrees, or between 130 and 135 degrees, or between 135 and 140 degrees, or between 140 and 145 degrees, or between 145 and 150 degrees, or between 150 and 180 degrees.

In some embodiments, the relative motion is between 0 and 10 microns per second, or between 10 and 20 microns per second, or between 20 and 50 microns per second, or between 50 and 100 microns per second, or between 100 and 200 microns per second, or between 200 and 500 microns per second, or between 500 and 1000 microns per second, or 1000 microns per second and higher.

In some embodiments, the separation distance (1028) between the surface of the open fluidic device and the blade is less than 10 microns, or less than 20 microns, or less than 30 microns, or less than 50 microns, or less than 75 microns, or less than 100 microns, or less than 200 microns, or less than 500 microns, or less than 1000 microns, or less than 2 mm, or less than 5 mm. In some embodiments, the separation distance is determined by a physical spacer that is positioned between the surface of the open fluidic device and the blade. In some embodiments, the physical spacer is integrated in the blade. In some embodiments, the physical spacer is integrated in the fluidic device.

In some embodiments, the blade comprises a fluidic device, in some embodiments, the blade comprises an open fluidic device. In some embodiments, the blade comprises fluidic features. In particular, the fluidic features may comprise open fluidic channels that improve control of the laminar flow of the solution of chromosomes onto the target surface to which said chromosomes will be positioned. In some embodiments, the blade comprises a substrate.

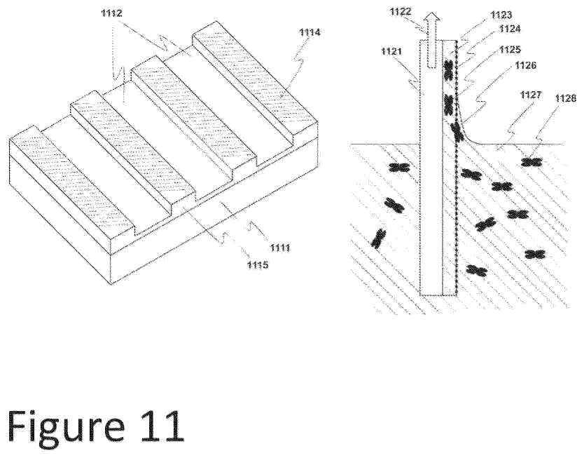

FIG. 11 demonstrates one particular embodiment of positioning chromosomes on a surface, whereby a solution of chromosomes are positioned on a surface with a dip coating method. In this particular drawn embodiment, the surface is the surface of an open fluidic device comprising fluidic features. The fluidic features are channels (1112) and walls (1115) patterned on a the surface of an open fluidic device (1111), whereby the top portion of the walls comprise a surface property that differs from the bottom of the channels (1112). In some embodiments, the property comprises surface energy. In some embodiments, the property comprises hydrophobicity. In some embodiments, the property comprises anti-fouling. In some embodiments, the property comprises surface roughness. In the preferred embodiment, as demonstrated in FIG. 11, the top portion of the walls are more hydrophobic than the bottom of the channels, however in some embodiments, the top portion of the walls are more hydrophilic than the bottom of the channels.

The chromosomes are positioned on the surface of the fluidic device by at least partially submerging the device (1121) in a solution (1127) of chromosomes (1128), and then extracting (1122) the device from the solution. As the device is extracted, a trailing solution meniscus (1126) forms on the surface of the device, leaving the hydrophilic channels (1123) wetted with the solution, while the top of the hydrophobic walls (1124) are unwetted, thus positioning the chromosomes (1125) in the channels.

In some embodiments, the angle at which the surface of the solution extends along the surface of the open fluidic device, and the relative direction of movement during operation is 90 degrees, or between 90 and 95 degrees, or between 95 and 100 degrees, or between 100 and 105 degrees, or between 105 and 110 degrees, or between 110 and 115 degrees, or between 115 and 120 degrees, or between 120 and 125 degrees, or between 125 and 130 degrees, or between 130 and 135 degrees, or between 135 and 140 degrees, or between 140 and 145 degrees, or between 145 and 150 degrees, or between 150 and 180 degrees.

In some embodiments, the angle at which the solution surface extends along the surface of the open fluidic device, and the major axis of the channels is between 90 and 95 degrees, or between 95 and 100 degrees, or between 100 and 105 degrees, or between 105 and 110 degrees, or between 110 and 115 degrees, or between 115 and 120 degrees, or between 120 and 125 degrees, or between 125 and 130 degrees, or between 130 and 135 degrees, or between 135 and 140 degrees, or between 140 and 145 degrees, or between 145 and 150 degrees, or between 150 and 180 degrees.

In some embodiments, the rate of extraction is between 0 and 10 microns per second, or between 10 and 20 microns per second, or between 20 and 50 microns per second, or between 50 and 100 microns per second, or between 100 and 200 microns per second, or between 200 and 500 microns per second, or between 500 and 1000 microns per second, or 1000 microns per second and higher.

In some embodiments, the surface onto which the chromosomes are positioned by dip coating does not comprise patterned features.

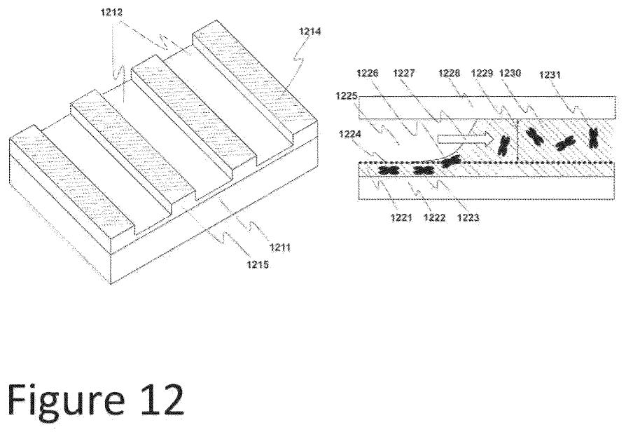

FIG. 12 demonstrates one particular embodiment of positioning chromosomes on a surface, whereby a solution of chromosomes are positioned on a surface with flow-cell coating. In this particular drawn embodiment, the surface is the surface of a closed fluidic device comprising fluidic features. The fluidic features are channels (1212) and walls (1215) patterned on the inner surface of a flow-cell of a closed fluidic device (1211), whereby the top portion of the walls comprise a surface property that differs from the bottom of the channels (1212). In some embodiments, the property comprises surface energy. In some embodiments, the property comprises hydrophobicity. In some embodiments, the property comprises anti-fouling. In some embodiments, the property comprises surface roughness, In the preferred embodiment, as demonstrated in FIG. 12, the top portion of the walls are more hydrophobic than the bottom of the channels, however in some embodiments, the top portion of the walls are more hydrophilic than the bottom of the channels.

The chromosomes are positioned on the surface of the fluidic device (1222) by flowing a solution (1230) of chromosomes (1231) through a fluidic flow cell channel (1225), said flow cell channel at least partially defined by a roof (1228). The solution is flowed in a direction (1227) such that a trailing meniscus is formed (1226), and said trailing meniscus removes the solution from the hydrophobic wall top (1224), while leaving the fluidic feature hydrophilic channels (1221) wetted with the solution and thus positioning the chromosomes (1223) in the channels.

In some embodiments, the angle at which the solution meniscus extends along the surface of the fluidic device flow-cell, and the direction of moving meniscus during operation is 90 degrees, or between 90 and 95 degrees, or between 95 and 100 degrees, or between 100 and 105 degrees, or between 105 and 110 degrees, or between 110 and 115 degrees, or between 115 and 120 degrees, or between 120 and 125 degrees, or between 125 and 130 degrees, or between 130 and 135 degrees, or between 135 and 140 degrees, or between 140 and 145 degrees, or between 145 and 150 degrees, or between 150 and 180 degrees.

In some embodiments, the angle at which the solution meniscus extends along the surface of the fluidic device flow-cell, and the major axis of the channels is between 90 and 95 degrees, or between 95 and 100 degrees, or between 100 and 105 degrees, or between 105 and 110 degrees, or between 110 and 115 degrees, or between 115 and 120 degrees, or between 120 and 125 degrees, or between 125 and 130 degrees, or between 130 and 135 degrees, or between 135 and 140 degrees, or between 140 and 145 degrees, or between 145 and 150 degrees, or between 150 and 180 degrees.

In some embodiments, the rate of meniscus movement along the surface of the flow-cell is between 0 and 10 microns per second, or between 10 and 20 microns per second, or between 20 and 50 microns per second, or between 50 and 100 microns per second, or between 100 and 200 microns per second, or between 200 and 500 microns per second, or between 500 and 1000 microns per second, or 1000 microns per second and higher

In some embodiments, the separation distance (1229) between the surface of the fluidic device flow-cell surface the roof is less than 10 microns, or less than 20 microns, or less than 30 microns, or less than 50 microns, or less than 75 microns, or less than 100 microns, or less than 200 microns, or less than 500 microns, or less than 1000 microns, or less than 2 mm, or less than 5 mm.

In some embodiments, the roof (1228) is detachable from the fluidic device (1222).

In some embodiments, the flow-cell surface onto which the chromosomes are positioned by flow-cell coating does not comprise patterned features.

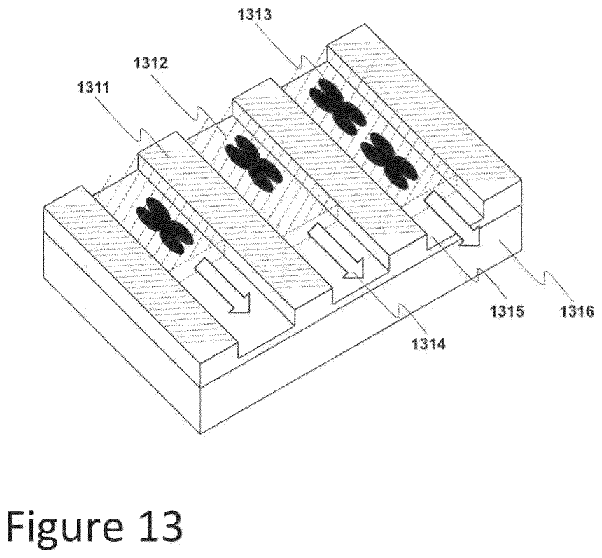

FIG. 13 demonstrates one particular embodiment of positioning chromosomes on a surface, whereby a solution of chromosomes are positioned on a surface with flow, in this example capillary flow. In this particular drawn embodiment, the surface is the surface of an open fluidic device comprising fluidic features. The fluidic features are channels (1315) and walls (1311) patterned on the surface of an open fluidic device (1316), whereby the top portion of the walls comprise a surface property that differs from the bottom of the channels. In some embodiments, the property comprises surface energy. In some embodiments, the property comprises hydrophobicity. In some embodiments, the property comprises anti-fouling. In some embodiments, the property comprises surface roughness. In the preferred embodiment, as demonstrated in FIG. 13, the top portion of the walls are more hydrophobic than the bottom of the channels, however in some embodiments, the top portion of the walls are more hydrophilic than the bottom of the channels.

The chromosomes are positioned on the surface of the fluidic device by flowing a solution (1312) of chromosomes (1313) through the channels (1315) via capillary flow (1314). The relatively more hydrophobic wall tops (1311) contain the solution in the channels during the capillary flow, thus positioning the chromosomes in the channels.

In some embodiments, the rate of flow through the channels is between 0 and 10 microns per second, or between 10 and 20 microns per second, or between 20 and 50 microns per second, or between 50 and 100 microns per second, or between 100 and 200 microns per second, or between 200 and 500 microns per second, or between 500 and 1000 microns per second, or 1000 microns per second and higher.

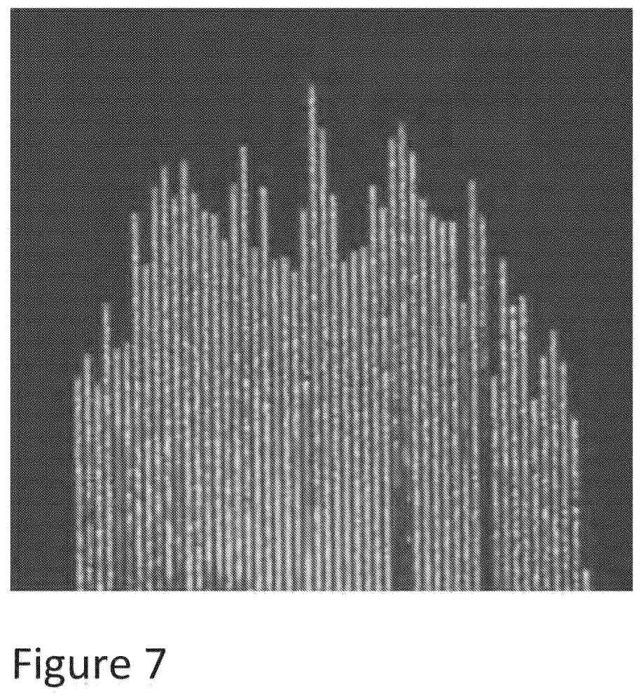

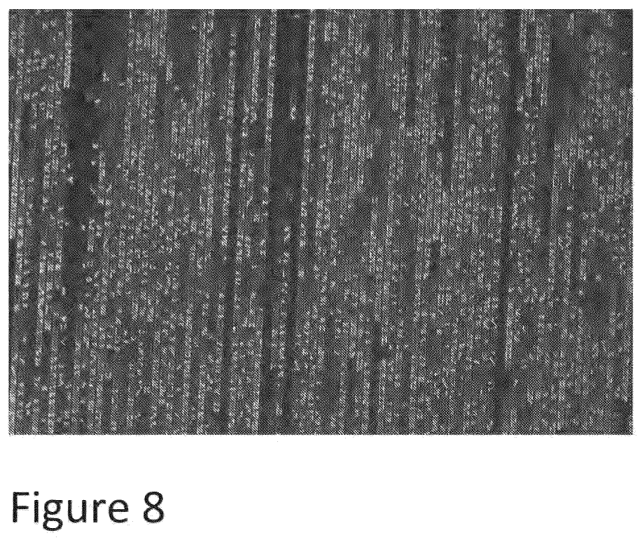

FIG. 7 is an example of chromosomes being flowed into an open fluidic device by capillary loading. In this particular example, a hydrophilic substrate is patterned with fluidic features that include hydrophobic channel walls, such that an aqueous solution containing chromosomes is contained within the channels by said channel walls. At the time of capture of this image, the solution is flowing from bottom to top by capillary forces. FIG. 8 is a similar open fluidic device as that shown in FIG. 7, however the image is captured after the capillary loading is complete, and the aqueous solution has evaporated, leaving the dry chromosomes confined within the channels on the surface of the device.

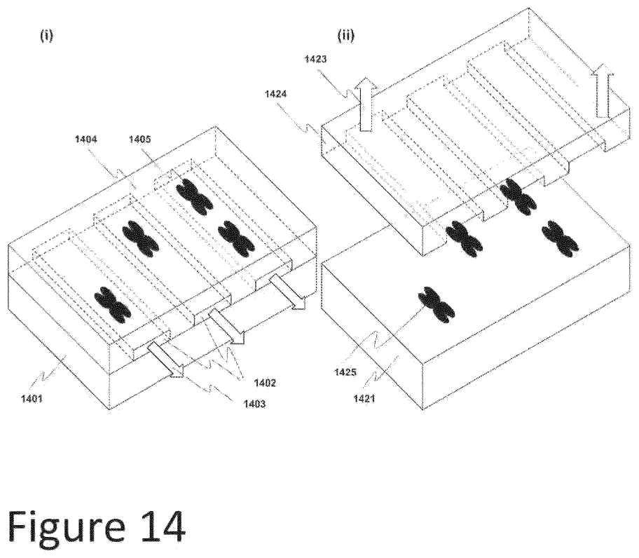

FIG. 14 demonstrates one particular embodiment of positioning chromosomes on a surface, whereby a solution of chromosomes are positioned on a surface with capillary flow within a fluidic device, where by the fluidic device includes a removable roof. In this particular drawn embodiment, the surface is the floor of the fluidic features (1402) (in this example the fluidic features are enclosed channels), whereby the enclosed channels are formed by the temporary bonding of the bottom substrate (1401) and the removable lid (1404), which together when bonded form the fluidic device. A solution containing chromosomes (1405) are flowed through the channels to locate the chromosomes within the channels. At a later point in time, the removable lid (1424) is detached (1423) from the substrate (1421), leaving the chromosomes (1425) positioned on the surface of the substrate.

In the example demonstrated in FIG. 14, the fluidic features (channels) were defined in the removable lid. In some embodiments, the fluidic features can be defined within the substrate. In some embodiments, the fluidic features can be defined in both the substrate and the removable lid. In some embodiments, at least one region of the removable lid and at least one region of the substrate share a property that differs from each other. In some embodiments this property is an anti-fouling property, or a hydrophobicity property, or a surface energy property, or a surface roughness property, or an optical property, or a surface functionalization property, or a material property, or a conductivity property. In some embodiments, the lid is removed after at least a portion of the solution has been removed from the channels. In some embodiments, the lid is removed after an interrogation of the chromosomes within the fluidic device.

In some embodiments, the rate of flow through the channels is between 0 and 10 microns per second, or between 10 and 20 microns per second, or between 20 and 50 microns per second, or between 50 and 100 microns per second, or between 100 and 200 microns per second, or between 200 and 500 microns per second, or between 500 and 1000 microns per second, or 1000 microns per second and higher.

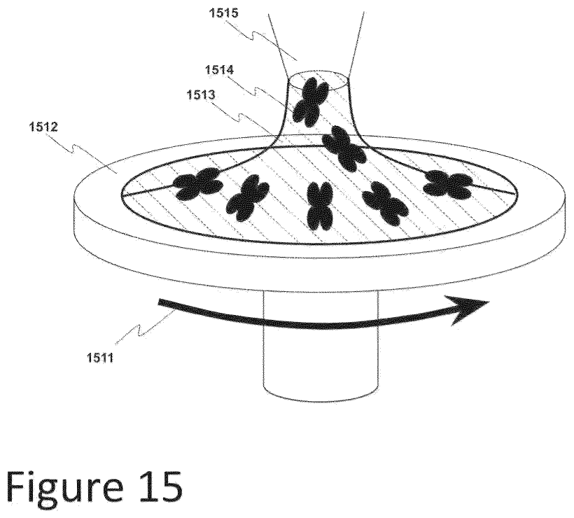

FIG. 15 demonstrates one particular embodiment of positioning chromosomes on a surface, whereby a solution of chromosomes are positioned on a surface of a substrate with spin coating. The chromosomes are positioned on the surface of the fluidic device by dispensing (1515) a solution (1513) of chromosomes (1514) onto a substrate (1512), which is then spun (1511) during and/or after the dispensing of solution. The centripetal force of the spinning substrate distributes the solution into a thin film of chromosomes on the surface of the substrate.

In some embodiments the substrate rotational speed comprises 0 -10 RPM, or 10-20 RPM, or 20-50 RPM, or 50-100 RPM, or 100-200 RPM, or 200-500 RPM, or 500-1000 RPM, or 1000-2000 RPM, or 2000-5000 RPM, or 5000-10000 RPM.

In some embodiments, the substrate comprises a fluidic device.

In some embodiments, the fluidic device is a closed fluidic device, and comprises a removable roof.

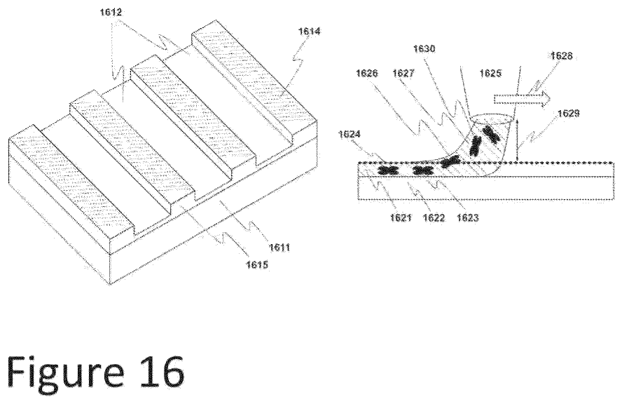

FIG. 16 demonstrates one particular embodiment of positioning chromosomes on a surface, whereby a solution of chromosomes are positioned on a surface of an open fluidic device by dispensing said solution with a dispensing system onto the surface of the open fluidic device. In this particular embodiment, the fluidic features are channels (1612) and walls (1615) patterned on the surface of an open fluidic device (1611), whereby the top portion of the walls comprise a surface property that differs from the bottom of the channels (1612). In some embodiments, the property comprises surface energy. In some embodiments, the property comprises hydrophobicity. In some embodiments, the property comprises anti-fouling. In some embodiments, the property comprises surface roughness. In the preferred embodiment, as demonstrated in FIG. 16, the top portion of the walls are more hydrophobic than the bottom of the channels, however in some embodiments, the top portion of the walls are more hydrophilic than the bottom of the channels.

The chromosomes are positioned on the surface of the fluidic device by bringing a dispensing system (1625) within proximity to the open fluidic device (1622), such that a desired distance (1629) is obtained between the dispensing system and the surface of the device, and with the dispensing tip positioned at a desired x-y coordinated above the surface of the device, whereby the xy plane is defined on the surface of the device. A solution (1627) containing chromosomes (1630) is then dispensed at the desired flow rate from the dispensing system onto the surface of the open fluidic device. In this particular drawn embodiment, the dispensing system is dispensing the solution while simultaneously moving (1628) the dispensing system relative to the xy coordinate system. In some embodiments, the dispensing system is at least partially maintained at a fixed position relative to the xy coordinate system during a portion of the dispensing.

As the dispensing system progresses dispensing the solution, a trailing meniscus (1626) forms from the dispensing system to the surface of the fluidic device, and the trailing meniscus removes the solution from the wall top (1624), while leaving the channel (1621) wetted with the solution and thus positioning the chromosomes (1623) in the channel.

In some embodiments, the solution dispensing comprises a continuous stream. In some embodiments, the solution dispensing comprises a dis-continuous stream. In some embodiments, the solution is dispensed only at particular xy coordinates. In some embodiments, the particular xy coordinates are at least partially determined by the position of certain fluidic features on the surface of the fluidic device. In some embodiments, the solution of chromosomes is flowed into fluidic features by capillary flow, whereby the flow originates from at least one fluidic well that is at least partially filled by the dispensing system.

In some embodiments, the relative motion between the dispensing system and the open fluidic device is between 0 and 10 microns per second, or between 10 and 20 microns per second, or between 20 and 50 microns per second, or between 50 and 100 microns per second, or between 100 and 200 microns per second, or between 200 and 500 microns per second, or between 500 and 1000 microns per second, or 1000 microns per second and higher.

In some embodiments, the separation distance (1629) between the surface of the open fluidic device and the dispensing system is less than 10 microns, or less than 20 microns, or less than 30 microns, or less than 50 microns, or less than 75 microns, or less than 100 microns, or less than 200 microns, or less than 500 microns, or less than 1000 microns, or less than 2 mm, or less than 5 mm, or less than 10 mm, or less than 100 mm.

In some embodiments, the surface onto which the chromosomes are positioned by dispensing does not comprise patterned features.

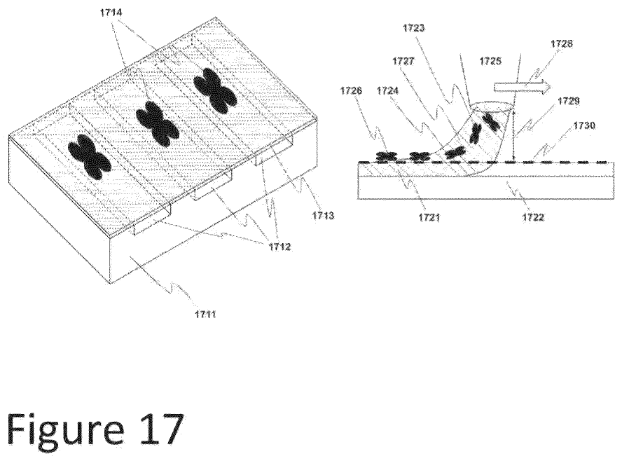

FIG. 17 demonstrates one particular embodiment of positioning chromosomes on a surface, whereby a solution of chromosomes are positioned on the surface of a porous roof defined within fluidic device by dispensing said solution onto the surface of said device. In this particular embodiment, the fluidic features are channels (1712) are defined within the surface fluidic device (1711), whereby a porous region (1713) forms both the roof of the channels, and the surface of the device. The porous region has a porosity property that allows solution to flow through it, while physically blocking the translocation of chromosomes of a certain property. In the preferred embodiment, the property is size, and the porous region blocks chromosomes larger than a certain size, as demonstrated in this particular drawn embodiment, whereby large chromosomes (1714) are positioned on top of the porous region. In some embodiments, the property is charge, and the porous region blocks chromosomes with a total charge, or charge density, greater than a certain value,

In this particular example, the chromosomes are positioned on the surface of the fluidic device by bringing a dispensing system (1725) within proximity to the open fluidic device (1722), such that a desired distance (1729) is obtained between the dispensing system and the surface of the device, and with the dispensing tip positioned at a desired x-y coordinated above the surface of the device, whereby the xy plane is defined on the surface of the device. A solution (1727) containing chromosomes (1723) is then dispensed at the desired flow rate from the dispensing system onto the surface of the open fluidic device. In this particular drawn embodiment, the dispensing system is dispensing the solution while simultaneously moving (1728) the dispensing system relative to the xy coordinate system. In some embodiments, the dispensing system is at least partially maintained at a fixed position relative to the xy coordinate system during a portion of the dispensing.

As the dispensing system progresses dispensing the solution, a trailing meniscus (1724) forms from the dispensing system to the surface of the fluidic device, and the trailing meniscus flows through the porous region surface (1730) into the channel (1721), leaving the chromosomes (1726) positioned on top of the porous region surface.

In some embodiments, the solution dispensing comprises a continuous stream. In some embodiments, the solution dispensing comprises a dis-continuous stream. In some embodiments, the solution is dispensed only at particular xy coordinates. In some embodiments, the particular xy coordinates are at least partially determined by the position of certain fluidic features on the surface of the fluidic device. In some embodiments, the solution of chromosomes is flowed into fluidic features by capillary flow, whereby the flow originates from at least one fluidic well that is at least partially filled by the dispensing system.

In some embodiments, the chromosomes are positioned on the surface of the porous region surface by any of the other previously mentioned deposition methods. In particular the methods can include combing, blade coating, patterned-blade coating, bar coating, solution shearing, slot-die coating, zone casting, knife coating, wicking, flow coating, flow-cell coating, dispensing, dropping, spin coating, off-center spin coating, casting, edge casting, sandwich casting, dispensing, spraying, tape coating, dip coating, meniscus coating.

In some embodiments, the surface onto which the chromosomes are positioned does not comprise patterned features.

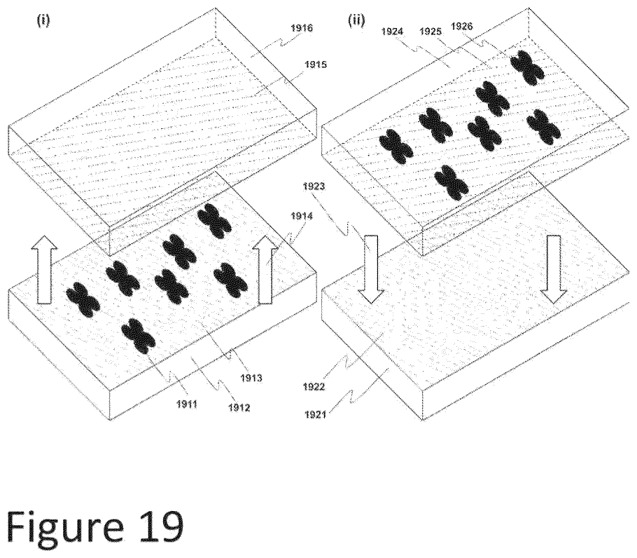

FIG. 19 demonstrates one particular embodiment of positioning chromosomes on a surface, whereby chromosomes are first positioned on the surface of an intermediate substrate, and then said chromosomes are transferred to the target surface by transfer printing. In this particular drawn embodiment, an intermediate substrate (1912) has chromosomes (1911) positioned on the surface (1913), and this intermediate substrate is brought (1914) into direct contact with the target surface (1915) of a substrate or fluidic device (1916). After contact, the chromosomes (1926) are transferred to the target surface (1925) due to said chromosomes having a greater affinity for being immobilized on said target surface, and removing (1923) the intermediate substrate (1921) from contact with the target substrate or fluidic device (1924), leaving the surface of the intermediate substrate (1922) free of chromosomes, or with a reduced number of chromosomes.

In the preferred embodiment, the intermediate substrate is deformable or malleable. In some embodiments, the intermediate substrate comprises silicone. In some embodiments, the target substrate or intermediate substrate or both comprise fluidic features or patterned features.

### Surfaces

The following describes a collection of surface embodiments, onto which the target molecule such as chromosomes are positioned. In some embodiments, the surface is a surface within a fluidic device. In some embodiments, the surface is a surface within or on an open fluidic device. In some embodiments, the surface is a surface of a substrate. In some embodiments, at least a portion of the surface is patterned. In some embodiments, at least a portion of the surface is un-patterned. In some embodiments, at least a portion of the surface comprises fluidic features. In some embodiments, at least a portion of the surface comprises a film.

In some embodiments, the patterned fluidic features may comprise patterned topologies or patterned surface energy regions that are patterned with at least one confining dimension suitably sized to accommodate at least a single macro-molecule. In some embodiments, the patterned fluidic features may comprise patterned topologies or patterned surface energy regions that are patterned with at least one confining dimension suitably sized to accommodate only one single macro-molecule. In some embodiments, the at least one confining dimension is a length dimension, a width dimension, a depth dimension, a height dimension, cross-sectional dimension, a radius dimension, a diameter dimension, an average distance between two planes dimension.

In some embodiments, the patterned fluidic features may comprise at least one confining or non-confining dimension that is constant over a length scale, or changes randomly over a length scale, or changes due to some design intent over a length scale on the surface of the fluidic device. In some embodiments, the patterned fluidic features may comprise a regular period or a random layout in their arrangement on the fluidic device surface.

For embodiments whereby the patterned fluidic features comprise a patterned topology, examples of patterned topologies include: open channels (isolated, connected, networked), pits, pillars, cones, corners, chevrons, branches, obstacles, pinch-points, forks, or intersections. In some embodiments, the cross-sectional profile of the topology may be square, rectangular, polygon, triangular, rounded, semi-oval, or semi-circular.

For embodiments whereby the patterned fluidic features comprise a patterned surface region, examples of pattern surface regions include: lines, rectangles, circles, hexagons, squares, open channel walls, open channel floors, open channel edges, open channel corners, pit walls, pit floors, pit edges, pillar bases, pillar walls, pillar roofs, pillar edges, cone walls, cone spikes. In some embodiments the surface region may be flat, or contoured, or have corners, or have edges.

The patterned fluidic feature may comprise an at least one confining dimension that is approximately 25% the length of said macro-molecule's axes, or is approximately 30% the length of said macro-molecule's axes, or is approximately 35% the length of said macro-molecule's axes, or is approximately 40% the length of said macro-molecule's axes, or is approximately 45% the length of said macro-molecule's axes, or is approximately 50% the length of said macro-molecule's axes, or is approximately 55% the length of said macro-molecule's axes, or is approximately 60% the length of said macro-molecule's axes, or is approximately 65% the length of said macro-molecule's axes, or is approximately 70% the length of said macro-molecule's axes, or is approximately 75% the length of said macro-molecule's axes, or is approximately 80% the length of said macro-molecule's axes, or is approximately 85% the length of said macro-molecule's axes, or is approximately 90% the length of said macro-molecule's axes, or is approximately 95% the length of said macro-molecule's axes, or is approximately 100% the length of said macro-molecule's axes, or is approximately 105% the length of said macro-molecule's axes, or is approximately 110% the length of said macro-molecule's axes, or is approximately 115% the length of said macro-molecule's axes, or is approximately 120% the length of said macro-molecule's axes, or is approximately 125% the length of said macro-molecule's axes, or is approximately 130% the length of said macro-molecule's axes, or is approximately 135% the length of said macro-molecule's axes, or is approximately 140% the length of said macro-molecule's axes, or is approximately 145% the length of said macro-molecule's axes, or is approximately 150% the length of said macro-molecule's axes, or is approximately 160% the length of said macro-molecule's axes, or is approximately 170% the length of said macro-molecule's axes, or is approximately 180% the length of said macro-molecule's axes, or is approximately 190% the length of said macro-molecule's axes, or is approximately 200% the length of said macro-molecule's axes, or is approximately 250% the length of said macro-molecule's axes, or is approximately 300% the length of said macro-molecule's axes, or is approximately 500% the length of said macro-molecule's axes.

The patterned fluidic feature may comprise an at least one confining dimension that is approximately 0.1-0.2 microns, or 0.2-0.3 microns, or 0.3-0.4 microns, or 0.4-0.5 microns, or 0.5-0.6 microns, or 0.6-0.7 microns, or 0.7-0.8 microns, or 0.8-0.9 microns, or 0.9-1.0 microns, or, 1.0-1.1 microns, or 1.1-1.2 microns, or 1.2-1.3 microns, or 1.3-1.4 microns, or 1.4-1.5 microns, or 1.5-1.6 microns, or 1.6-1.7 microns, or 1.7-1.8 microns, or 1.8-1.9 microns, or 1.9-2.0 microns, or 2.0-2.2 microns, or 2.2-2.4 microns, or 2.4-2.6 microns, or 2.6-2.8 microns, or 2.8-3.0 microns, or 3.0-3.5 microns, or 3.5-4.0 microns, or 4.0-4.5 microns, or 4.5-5.0 microns, or 5.0-6.0 microns, or 6.0-7.0 microns, or 7.0-8.0 microns, or 8.0-9.0 microns, or 9.0-10 microns.

In some embodiments, the patterned fluidic features may comprise a multitude of patterned topologies or patterned surface energy regions, wherein a variety or range of different confining dimensions are suitably sized to accommodate a variety or range of differently sized macro-molecules.

In the embodiments whereby the at least two macro-molecules comprise chromosomes, or more preferably meta-phase or pro-phase human chromosomes, the at least one confining dimension is between 0.25 to 10 microns, or more preferably between 0.5 to 5 microns. For example, a single human meta-phase chromosome that is approximately 1.5 micron in width, 1.5 micron in height, and 10 microns in length could be accommodated in an open channel that is 2 microns wide, 2 micron deep, and 50 microns in length.

In some embodiments, the patterned fluidic features may comprise patterned topologies or patterned surface energy regions that are patterned of a size to accommodate multiple macro-molecules in a single-file fashion. For example, a single-file row of 10,000 meta phase chromosomes, each approximately 10 microns in length, with 10 microns spacing the chromosomes would require a channel 200 mm in length.

In some embodiments, the patterned fluidic features may comprise of patterned topologies or patterned surface energy regions that are patterned of size to accommodate a macromolecule, a chromosome, or a long nucleic acid molecule in at least a partially elongated state, and thus comprises at least one confining dimension that is between 5-1000 nm, or more preferably, 10-500 nm. In some embodiments, the patterned fluidic features may comprise of patterned topologies or patterned surface energy regions that are patterned of a size to accommodate a macromolecule, a chromosome, or a long nucleic acid molecule in at least a partially elongated state, and thus comprises at least one non-confining dimension that is between 10-5000 microns, or more preferably, 25-2000 microns. For example, a single S mega-base length long nucleic acid molecule fully elongated can be accommodated in a single open channel of length 2000 microns.

In some embodiments, the patterned fluidic features may comprise of patterned topologies or patterned surface energy regions that are patterned of size to accommodate at least one ecDNA, and thus comprise at least one confining dimension that is between 0.1 to 5.0 microns, or more preferably 0.2 to 2.0 microns. For example, a 5 megabase ecDNA in a compact circular state may range in size from 0.01 to 2.0 micron in diameter, more typically from 0.1 to 1.0 micron in diameter.

FIG. 20 demonstrates a cross section SEM of an open channel fluidic device composed of silicon with 2 micron wide channels that are 1 micron deep and separated from each other by 2 microns. In the preferred embodiment, the surface of the barriers separating the channels is made hydrophobic or anti-fouling via chemical modification of the surface.

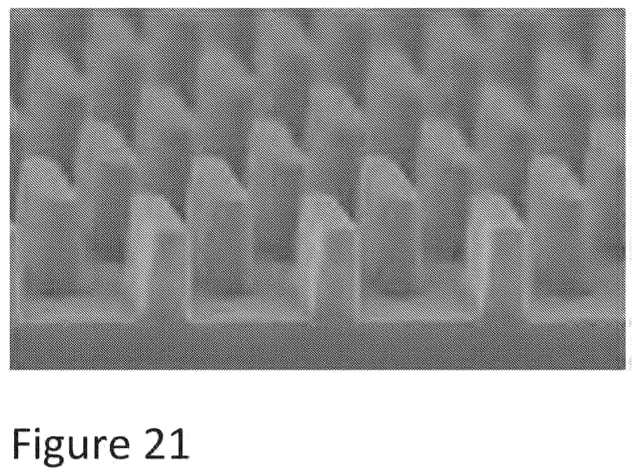

FIG. 21: demonstrates a cross section SEM of an open channel fluidic device composed of silicon pillars. In the preferred embodiment, the surface of the barriers separating the channels is made hydrophobic or anti-fouling via chemical modification of the surface.

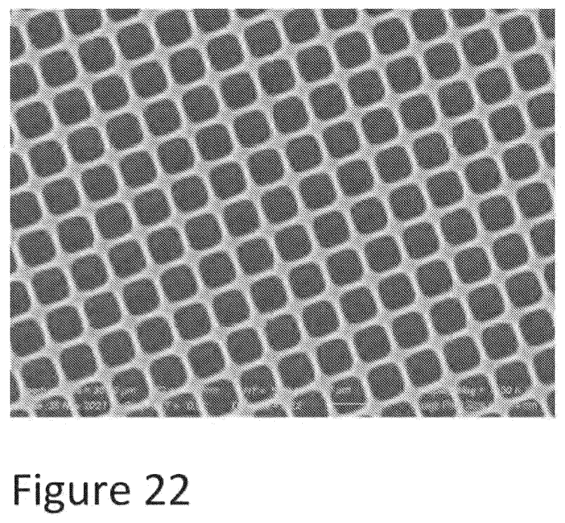

FIG. 22 demonstrates a top-down SEM image of an open pit fluidic device composed of silicon with 0.8 micron wide pits that are 1.0 micron deep. In the preferred embodiment, the surface of the barriers separating the channels is made hydrophobic or anti-fouling via chemical modification of the surface.

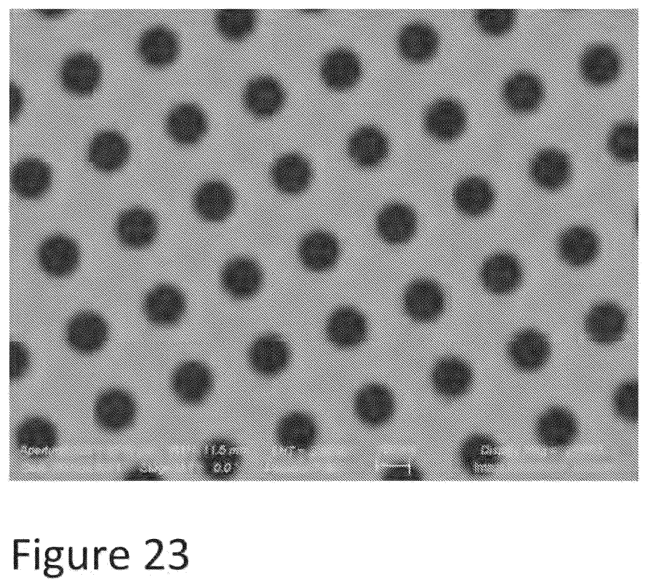

FIG. 23 demonstrates a top-down SEM image of an open pit fluidic device composed of silicon with 0.3 micron wide pits that are 1.0 micron deep In the preferred embodiment, the surface of the barriers separating the channels is made hydrophobic or anti-fouling via chemical modification of the surface.

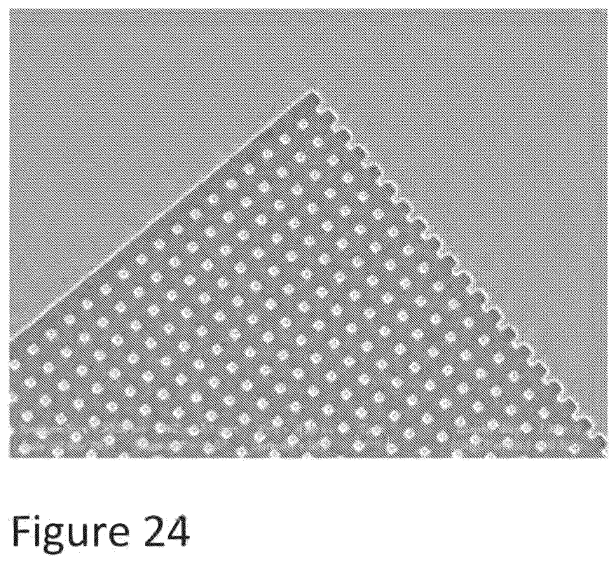

FIG. 24 demonstrates a top-down SEM image of an open pit fluidic device composed of silicon with 0.8 micron wide pillars that are 1.0 micron deep. In the preferred embodiment, the surface of the barriers separating the channels is made hydrophobic or anti-fouling via chemical modification of the surface.

In some embodiments, the patterned fluidic features may comprise patterned topologies or patterned surface energy regions that are patterned with at least one confining dimension suitably sized to exclude at least a single macro-molecule. In some embodiments, the at least one confining dimension is a length dimension, a width dimension, a depth dimension, a height dimension, cross-sectional dimension, a radius dimension, a diameter dimension, an average distance between two planes dimension. For example, an open fluidic channel with a width of 0.1 micron such that a human meta-phase chromosome is unable to be physically accommodated by the channel.

In some embodiments, the patterned fluidic features may comprise patterned topologies or patterned surface energy regions that are patterned with at least one confining dimension suitably sized to exclude at least a single macro-molecule of type A, and accommodate at least a single macro-molecule of type B, wherein type A and type B are macro-molecules of different sizes. In some embodiments, the at least one confining dimension is a length dimension, a width dimension, a depth dimension, a height dimension, cross-sectional dimension, a radius dimension, a diameter dimension, an average distance between two planes dimension. For example, an open fluidic channel with a width of 0.1 micron such that a human meta-phase chromosome is unable to be physically accommodated by the channel, however an ecDNA is able to be accommodated by the channel.

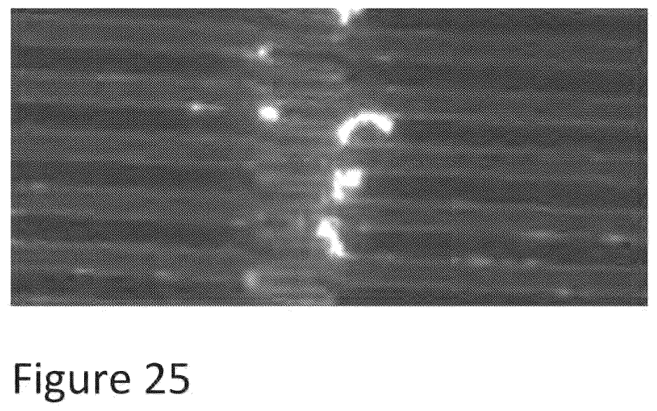

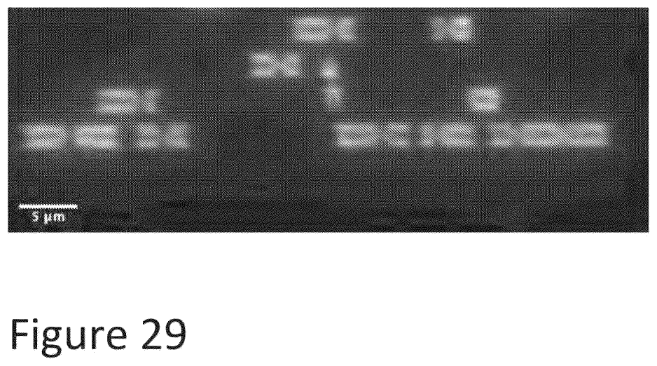

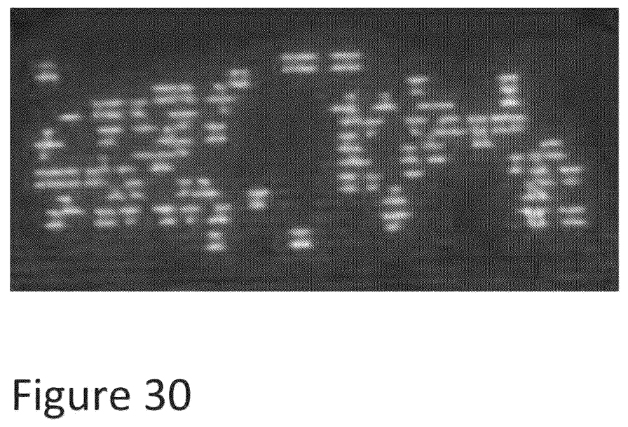

For purposes of staining, washing and further manipulation of nucleic acids, chromatin or chromosomes on surface, it is advantageous that this material stick firmly to the surface, preferably in a semi-flexible and chemically reversible manner. Chemical intuition can provide a starting point for speculating how exactly nucleic acids, chromatin and chromosomes dry on a surface and remain immobilized, particularly with the concepts of ionic interactions, charge screening, pH modification of charged groups and hydrophobic van der Walls forces, but the descriptions of methods that follow are not bound by theory. In some embodiments, nucleic acids are fixed to the surface by methods including but not limited to pH-dependent adhesion of nucleic acids to glass, interaction of nucleosides with a hydrophobic or moderately hydrophobic surface, interaction of phosphate backbone with a positively charged surface, interaction of phosphate backbones with a negatively charged surface in combination with positively charged bridging molecules, conformal drying of chromatin material onto a rough surface, capillary force pulling chromatin and chromatids into a sharp corner as seen in FIG. 29, FIG. 30, FIG. 25 and FIG. 37, Velcro-like interaction of protruding loops of nucleic acids or chromatin hooking on rough or nanopatterned or micropatterned surface features, solvent exclusion, liquid-liquid phase separation, non-covalent interaction of proteins with fluidic surface features, covalent interactions of nucleic acids and attached proteins with fluidic features including reactions of primary and secondary amines with glycidyl ethers including unreacted glycidyl ethers in photomasks and non-specific acid-catalyzed and radical catalyzed reactions of partially-reacted photomask materials with yet uncharacterized reactive moieties of chromatin, reaction of sulfhydryl groups and primary and secondary amines with chemical crosslinkers attached to the surface, not limited to poly glycidyl methacrylate, methacrylated matrices with unreacted methacrylate moieties including but not limited all methacrylate and acrylate polymers described herein, and interactions of the chromosome fixatives described herein with the surface chemical features of the device, preferably formaldehyde fixation of chromatin to a layer of gelatin methacrylate affixed to the surface. In further embodiments, semi-flexible mounting is achieved by placing nucleic acids, chromatin or chromosomes upon a monolayer, layer, oligomeric network or branched oligomeric network of polymers recognized for antifouling properties, preferably Poly[oligo(ethylene glycol) methyl ether methacrylate].

FIG. 25: an example of chromosomes being caught in a channel pinch point.

In some embodiments, the patterned fluidic features may comprise patterned topologies or patterned surface energy regions that are patterned with at least one fluidic feature suitably sized to capture at least a single macro-molecule of type A, and exclude at least a single macro-molecule of type B, wherein type A and type B are macro-molecules of different sizes. In some embodiments, the at least one fluidic feature is a channel, or a corner, or a pit, or a trench, or an edge.

In some embodiments, the patterned fluidic features designed to capture, exclude, or accommodate at least a single macro-molecule of type A is used to further analyze or process the at least a single macromolecule or chromosome of type A. In some embodiments, the analysis may comprise interrogation. In some embodiments, the analysis may comprise interaction with a contact probe. In some embodiments, the analysis may comprise interaction with a dispensing system. In some embodiments, the processing may comprise a chemical reaction with at least one reagent, or at least one enzyme. In some embodiments, the interrogation may comprise an analysis of the number or genomic state of the type A chromosomes. In some embodiments, the interrogation may comprise an analysis of the number or genomic state of the type B chromosomes.

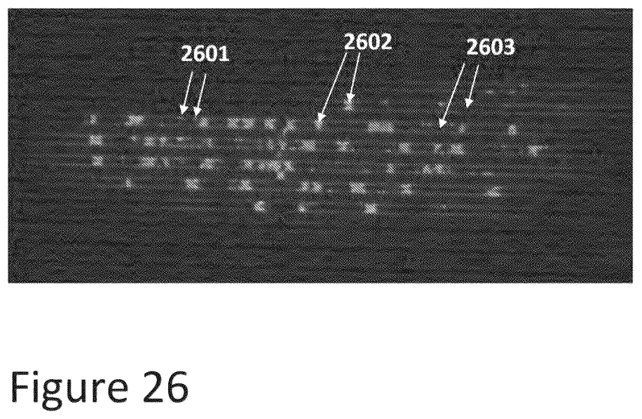

For example, FIG. 26 demonstrates an open fluidic channel (2603) with a width of 2 microns and a depth of 2 microns whereby human metaphase chromosomes (2602) and ecDNA (2601) originating from a single cell are accommodated by the channels. Here, the smaller ecDNA are further separated from the chromosomes and isolated along the bottom corners of the channels due to the surface tension force of the evaporating cell fluid in the channel, while the larger chromosomes experience surface tension forces from both bottom corners of the channels, and thus remain centered within the channels.

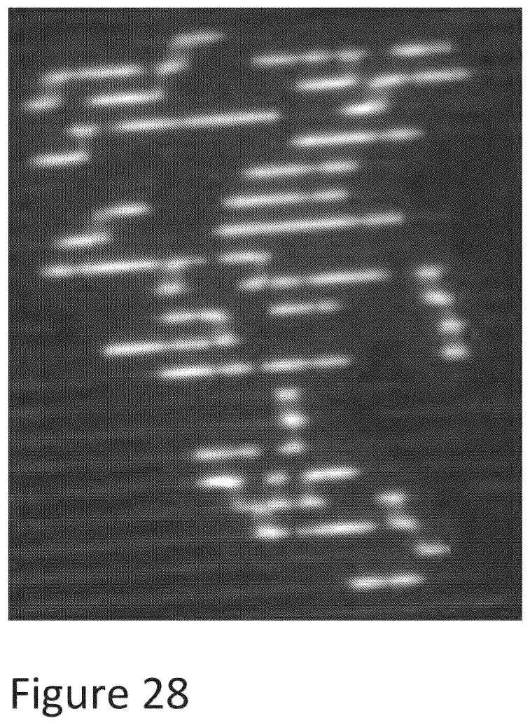

In some embodiments, the fluidic features comprise channels narrow enough to elongate the chromosomes and artificially increase their length, illustrated in FIG. 28. The problem of generating long chromosomes on a bare glass slide bas been a difficult problem and although several elegant cell biological and biochemical means to prepare long chromosomes exist, they exhibit high variability across different cell lines and can require considerable biochemical expertise to master, making them impractical for routine analysis.

In some embodiments, the width of the channels is larger than the natural separation between sister chromatids, and chromatids are pulled apart as illustrated in FIG. 29. This makes it easier to identify the centromere of a chromosome, or multiple centromeres in the case of aberrant chromosomes with duplicated centromeres Cell biological techniques and biochemical techniques to lengthen chromosomes often make it difficult to determine the exact position of the centromere, leading to uncertainty in the interpretation of standard karyograms, but that problem is lessened by suitable channel design. In other embodiments, centromeres are made easier to identify by deposition of chromosomes inside channels with channel widths larger than the natural width of a chromosome (FIG. 29), resulting in a pronounced X pattern around the centromere. The centromere is further emphasized by the fact that chromatin approaching the centromere no longer has the straight border that. chromatids have when they are in direct contact with the walls. The stretching force of the channels also results in lower density of chromatin around the centromere, providing a reduction in stain density in the centromere region, further enhancing its signal.

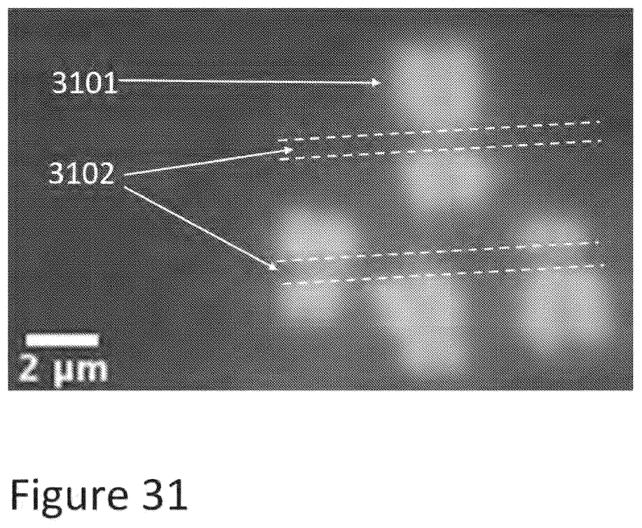

Chromosome identification procedures as used in clinical aneuploidy detection start with the classification of chromosomes according to centromere position as described in Arsham, Marilyn S., Margaret J. Barch, and Helen J. Lawce. The AGT Cytogenetics Laboratory Manual. 4th ed. Hoboken, New Jersey: Wiley-Blackwell, 2017, sorting chromosomes according to whether they are metacentric (centromere in the middle), submetacentric (centromere a little away from the middle so that one arm is longer than the other), acrocentric (centromere close to one end), or telocentric (centromere right at the end). In some embodiments, features in the fluidic device present narrow channel walls that position chromosomes such that sister chromatids are present in adjacent channels (FIG. 30) and centromeres are visualized as chromatin density that crosses across the narrow bars. In some embodiments, features in the fluidic device aid the classification process by providing narrow, low features that match the approximate dimensions of the chromosomal centromeres. For example, FIG. 31 demonstrates an embodiment whereby a chromosome (3101) is positioned with the centromere on a narrow bar (3102), while the arms of the chromosome are in channels of the fluidic device. In some embodiments, chromosomes do not interact with the walls of the device (FIG. 4).

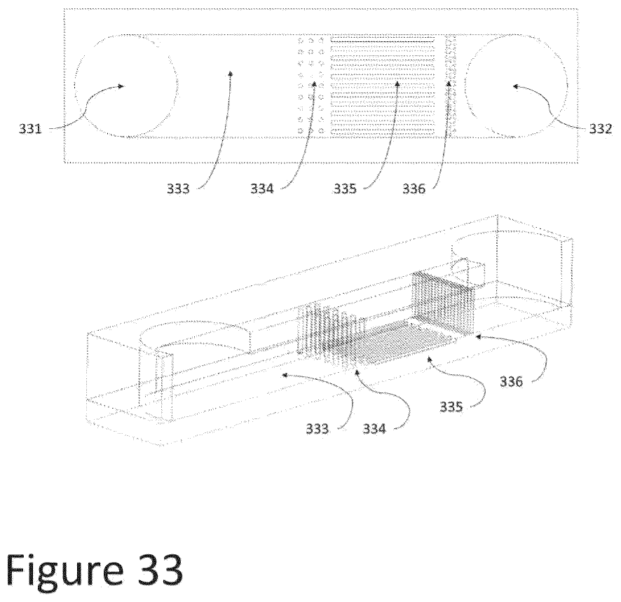

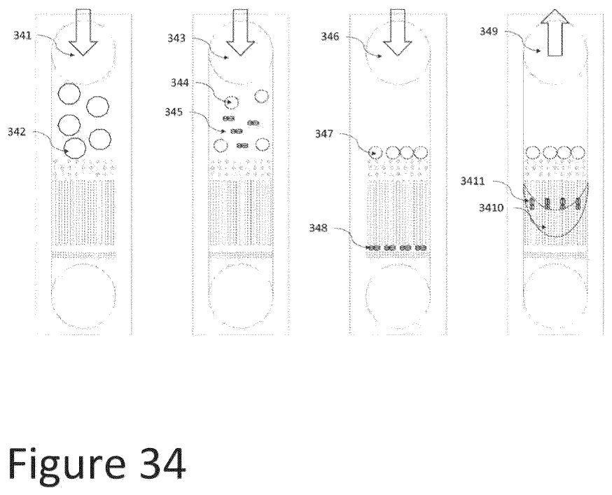

In some embodiments, the manipulation of cells and purification of chromosomes can occur within a fluidic, or preferably microfluidic, device that contains a surface upon which chromosomes will ultimately be deposited (FIG. 33, FIG. 34). In further embodiments, the fluidic device can also be used to apply chromosome condensation reagents such as Colcemid or Calyculin A to a cell while the cell is still alive, and in further embodiments the device can be used to culture and amplify cells for one or more generations. In some embodiments, a fluidic device can be assembled with a multiplicity of regions that each limit the number of cells that can contribute chromosomes to an adjacent interrogation region, such that the collection of chromosomes in an interrogation region can be reasonably assumed to have derived from one of a limited number of cells contributing to that region. In an embodiment that contains a cell deposition region small enough that only a single cell can fit in the region, or small enough that given an average proportion of cells that are in mitosis the chance of finding multiple mitotic cells within the region is <75% the data are consistent with single cell karyotyping and cases where multiple mitotic cells were present can be excluded without serious degradation of the overall throughput. In an embodiment where between 1 and approximately 10 mitotic cells are expected to be found on average, the data can be analyzed for aneuploidy of the sum of all cells, under the assumption that it is extremely unlikely that one cell has lost a particular chromosome and another cell has gained the same chromosome, thus causing an appearance of all cells being healthy. In an embodiment whereby the device and sample preparation together yield more than approximately 10 mitoic cells, more sophisticated models of ancuploidy are required to understand the mixture of chromosomes that are analyzed together.

In some embodiments, a fluidic device similar to the device described in FIG. 33 and FIG. 34 can be made with an additional interrogation chamber 335 and pillars 336 that are sized to discriminate between chromosomes of different sizes. In the preferred embodiment, the first set of 336 type pillars have a spacing of between 1.1 and 0.5 microns, most preferably 0.8 microns and arrayed in a hexagonal pattern, to permit ecDNA to pass through to the secondary interrogation chamber while larger chromosomes are confined to the first interrogation chamber. In further embodiments, additional sets of interrogation chambers and pillars filter chromosomes to finer degrees.

In some embodiments, a fluidic device similar to the device describe in FIG. 33 and FIG. 34 is comprised of two parallel surfaces upon which chromosomes are deposited. In some embodiments, only one chromosome deposition surface exists, and the alternate surface has a surface character or surface energy that is less favorable to the deposition buffer than the chromosome deposition surface and the meniscus that forms during deposition and evaporation is slanted as a result in order to selectively pull chromosomes towards the interrogation surface. In some embodiments, the walls of the fluidic device are coated in a similar fashion in order to direct chromosome deposition away from the walls. In some embodiments, channels are angled to deflect chromosomes away from the walls and do not extend the full distance across the width of the fluidic device.

In the preferred device embodiment, the fluidic features comprise regions of different surface energies. In particular, in the preferred device embodiment, the most elevated regions comprise a surface energy that is relatively hydrophobic with respect to the most depreciated regions. In another preferred device embodiment, the most elevated regions comprise a surface energy that is relatively antifouling with respect to the most depreciated regions.

In some embodiments, the channels are separated from adjacent and substantially parallel channels with a distance that can range from 0.2 to 100 microns. In some embodiments, the channels are patterned at fixed period. In some embodiments, the channels are patterned randomly. In some embodiments, the patterned features are at least partially comprising of pits or pillars. In some embodiments, the depth of the patterned features, or the height of the patterned features can range from 0 nm to 10 microns. (0 depth for embodiments where-by the channel is defined only by surface energy features.) In some embodiments, the patterned features are grouped in sets, in which they are physically co-located on the surface of the fluidic device.

In some embodiments whereby the fluidic device comprises a channel or chamber with a porous roof, the porous roof may comprise a silicon oxide, or silicon, or silicon nitride, or ITO, or glass, or quartz, or spin-on glass, or metal, or an oxidized metal, or a gel, or a polymer, or a cross-linked polymer. In some embodiments, the porous roof is manufactured with a process that comprises partially etching through a roof material until a certain level of porosity is achieved. Etching methods may comprise wet etching, acid etching, chemical etching, plasma etching, ion beam etching, reactive ion etching, inductively coupled plasma etching, sand blasting, focused ion beam etching. In some embodiments, the porous roof is manufactured with a process that comprises partially dissolving the roof material in a solvent. In some embodiments, the porous roof is manufactured by selectively removing a sacrificial material under the porous roof to form a channel or chamber under said roof. In the preferred embodiment, the sacrificial material is etched away with a solution, or dissolved away with a solvent. In some embodiments, the porous roof is manufactured with a process that comprises a degree of self-assembly. For example: self assembly of nano particles, or self-assembly of micro particles, or self-assembly of nanorods or nanotubes, or self-assembly of beads, or self-assembly of polymer cross-linking. In some embodiments, the porous roof is manufactured by selectively controlling the cross-linking density of a polymer, such that the regions of low cross-linking density are porous, and the regions of high cross-linking density are non-porous. In some embodiments, the porous roof is manufactured by depositing or growing material over a channel or chamber and accumulating sufficient material to form a porous structure over the chamber or channel.

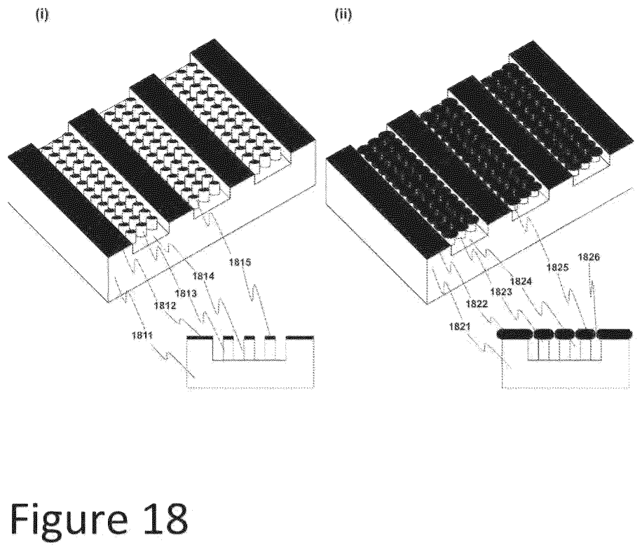

FIG. 18 demonstrates a particular embodiment of fabricating a fluidic device with a porous roof, whereby the porous roof comprises grown nickel deposits from a pattern array of seeds. In this drawn embodiment, a substrate is patterned with channels (1814), in which there are pillars (1813). Both between the channels (1812) and on top of the pillars (1815) there is a seed layer a material to support the deposition of Nickel via electroless growth. After the deposition, nickel films have formed (1822, 1825), growing up and out from the seeds. The result is nickel blobs on top of the pillars (1823), however the growth is stopped before completely sealing the roof, resulting in pores (1826) between the nickel blobs which allow for the formation of a porous roof over the channel (1824) of the fluidic device (1821). For a review on nickel plating micro-nanostructures, see Ahn, Jinseong, Seokkyoon Hong, Young-Seok Shim, and Junyong Park. “Electroplated Functional Materials with 3D Nanostructures Defined by Advanced Optical Lithography and Their Emerging Applications.” Applied Sciences 10, no. 24 (Dec. 8, 2020): 8780. In some embodiments the deposition process for generating the porous roof comprises sputtering, or evaporation, or thermal evaporation, or electron-gun evaporation, or electro-chemical growth, or spray coating, or spin coating, or lamination. In some embodiments, the porous roof comprises an adhesive film with porous properties.

In some embodiments, the surface of the porous roof is further modified In some embodiments the modification comprises functionalization, or a modification in surface energy, or a modification in the surface's anti-fouling properties, or a modification in charge density.

### Staining

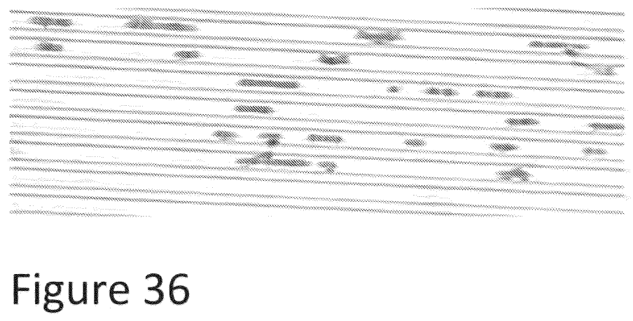

After chromosomes are positioned on a device, they can be imaged directly with methods that do not require labeling, or they can be stained using one or more of the full and rich variety of methods described in Arsham, Marilyn S., Margaret J. Barch, and Helen J. Lawce. The AGT Cytogenetics Laboratory Manual. 4th ed. Hoboken, New Jersey: Wiley-Blackwell, 2017, and known to those with skill in the art of cytogenetics, including without limitation Giemsa banding (G-bands), G-bands by trypsin using Giemsa (GtG) demonstrated in FIG. 36, Quinacrine banding (Q-bands), C-banding (C bands), G-11 staining, Centromere/kinetochore staining, Reverse banding (R-bands), DAPI/distamycin A staining (DA-DAPI), staining with a stain and a competitive binder, Silver staining (AgNOR) for nucleolus organizing regions, Replication banding using nucleotide analogues including 5-bromo-2′-deoxyuridine (BrdU) and 5-ethynyl-2′-deoxyuridine (EU), Sister chromatid exchanges stained with replication banding methods, T-banding/CT-banding, Antibody banding, Restriction endonuclease banding and FISH DAPI bands, Q-bands (Q), Q-bands by fluorescence (QF), Q-bands by fluorescence using quinacrine Q-bands by fluorescence using hoechst 33258 (GFh), G-bands (G), G-bands by trypsin (Gt), G-bands using Leishman (GtL), G-bands using Wright stain (GtW), G-bands using pancreatin and Giemsa stain (GpG), G-bands using urea and Giemsa stain (GUG), G-bands by acetic saline using Giemsa (GaG), C-bands (C), C-bands by barium hydroxide (CB), C-bands by barium hydroxide using Giemsa (CBG), r-bands (r), r-bands by fluorescence (rF), r-bands by fluorescence using acridine orange (rFa), r-bands by heating (rh), r-bands by heating using Giemsa (rhG), r-bands by BrdU (rB), r-bands by BrdU using Giemsa (rBG), r-bands by BrdU using acridine orange (rBa), t-bands (t), t-bands by heating (th), t bands by heating with Giemsa (thG), t-bands by heating with acridine orange (tha). In some embodiments, chromosomes are fixed and washed prior to further staining and manipulation, preferably using fixatives described prior in the section describing fixation before, during or after lysis, but most preferably by immersion in an anhydrous mixture of 3:1 methanol:acetic acid for 5 minutes.

In some embodiments, phase-based optical methods are used to detect biological material, the methods including phase contrast, differential interference contrast, reflective differential interference contrast, Nomarski contrast, Hoffman contrast and related illumination strategies asymmetric with respect to the detection pupil, methods that employ defocused or multifocus based phase imaging including transport of intensity equation based methods. In the case where the fluidic structure comprises a regular array of channels that function as an optical grating, the presence of chromatin can be detected in some embodiments using directional excitation incident upon the grating to construct positive or negative interference in the absence of chromatin, whereby the presence of chromatin modulates the degree of interference to create contrast. In alternate embodiments, contact methods including atomic force microscopy contact the chromosomes or chromatin to read the shape.

The presence of cellular debris can be confused for chromosomes in some phase and contact based methods, and can hinder the accessibility of nucleic acids, chromatin and chromosomes in other assays that require access by chemicals, macromolecules and or particles. In some embodiments, chromosomes are cleaned using a mild detergent including the non-ionic detergents described herein, preferably Tween 20, a block-co-polymer containing at least one charged moiety, preferably PEI-g-PEG, RNAse, preferably RNAse A, or a protease, preferably pepsin, or most preferably both RNAse A and pepsin sequentially applied, followed by washing with a dilute aqueous buffer.

In some embodiments, chromosomes are stained with non-specific absorbing or fluorescent dyes, preferably dimeric intercalators, most preferably YOYO-1, and are imaged to determine physical morphology including the number of chromatids, degree of completion of the most recently replicated chromatid, outer dimensions of the chromosome, location of the centromere with respect to the overall length, separation distance between chromatids, stiffness of the chromosome or chromatids with respect to deformation against a structured surface or other particle upon the surface, degree of axial compaction of the chromatids, number and density of attachments between adjacent chromatids, and degree of nucleic acid detachment from the main core of the chromatin.

In some embodiments, multiple staining is performed and the chromosomes are interrogated and analyzed for multiple stains, the data being combined for each chromosome. FISH in particular is performed in concert with specific and non-specific nucleic acid or chromatin staining using fluorophores, preferably DAPI, and in certain embodiments FISH is performed with multiple colour probes simultaneously and imaged with a multichannel fluorescence microscopy system. In further embodiments, single or multicolour FISH is performed multiple times and imaged multiple times, with or without additional cycles to abrogate the signal of prior probes including without limitation denaturation with fluid flow, chemical bleaching of dyes, photobleaching of dyes and blank imaging cycles to verify prior probe removal or quenching.

Replication banding with unnatural nucleosides (Drouin 1991, Hoshi, Osamu, and Tatsuo Ushiki. “Replication Banding Patterns in Human Chromosomes Detected Using 5-Ethynyl-2′-Deoxyuridine Incorporation.” ACTA HISTOCHEMICA ET CYTOCHEMICA 44, no. 5(2011 ): 233-37) can be performed by adding an unnatural nucleoside metabolic label containing one half of a reactive group, including without limitation 5-azido-2′-deoxyuridine (AdU), 5-(azidomethyl)-2′-deoxyuridine (AmdU) or preferably an alkyne capable of CuAAC click chemistry such as 5-ethynyl-2′-deoxyuridine, to actively dividing cells. This is preferably performed while cells are synchronized, such as results from growth under the blocking presence of an excess of thymidine, and subsequently released from the block by the addition of the metabolic label. Chromosomes can be harvested at a specific time in the cell cycle, preferably late metaphase, or trapped in a condensed state as usual using Colcemid, also applied at a specific time in the cell cycle. The resulting chromosomes can be stained by combining with fluorophores, dyes, or haptens, each present with a complementary lableing moiety including without limitation azide, picoyl azide, alkyne, alkyne derivative, bicyclo[6.1.0]nonyne (BCN) or dibenzocyclooctyne group (DBCO), and incubated with the standard buffer conditions as practiced in the art to perform the coupling reaction.

In some embodiments, the deposited nucleic acids, preferably chromatin fragments and most preferably chromatin, are labeled with a labeling body that is attached to a matrix that is site-specifically synthesized around one or more specific nucleic acid sequences. A plurality of nucleic acid molecules are deposited on a fluidic surface, preferably a patterned fluidic surface, with minimal overlap, and fixed using methods described herein, preferably by immersion in a 3:1 mixture of anhydrous Methanol: Acetic Acid for 5 minutes followed by drying. In some embodiments, labeled FISH probes are brought into contact with the nucleic acids, the probes comprising nucleic acid or peptide-nucleic acid regions covalently attached to a label precursor moiety, preferably by a 5′ or 3′ linkage, and FISH probes are hybridized and washed. In some embodiments, FISH label precursors are catalytic in nature, including but not limited to the photosensitizers Atto Thio12, Bodipy, anthraquinone, and preferably Rose Bengal, and combined with a source of radiation provide a local source of reaction initiation sites that start polymerization of a monomer that is subsequently applied, preferably an acrylate or methacrylate containing molecule as described for surface chemistry modifications herein. In some embodiments, FISH label precursors directly contain an initiator molecule capable of polymerization, preferably controlled polymerization including but not limited to Atom Transfer Radical Polymerization (ATRP), reversible addition-fragmentation chain transfer polymerization (RAFT), or ring opening metathesis polymerization (ROMP), and after non-binding FISH probes are washed away, the labeled nucleic acids are combined with a monomer, preferably an acrylate or methacrylate containing molecule as described for surface chemistry modifications herein, and reaction conditions known to polymerize the monomer in a controlled manner respective to the technique used, the preferred technique being oxygen-tolerant ATRP. In some embodiments, FISH label precursors are affinity labeling bodies, preferably biotin or digoxigenin, and after non-bound FISH labels are washed way, the labeled nucleic acids are brought into contact with complementary labeling bodies, preferably streptavidin and anti-digoxigenin antibodies, that are in turn labeled with a secondary label. In some embodiments the secondary label is an initiator for controlled polymerization including but not limited to ROMP, RAFT and preferably ATRP, and following the stringent application of the secondary label the sample is subjected to the polymerization conditions of the respective methods and results in local formation of a polymer around the sequence of interest. In other embodiments the secondary labels are one or more catalytic labels as described above, while in yet other embodiments the secondary labels are polymerizable proteins or preferably enzymes, most preferably horseradish peroxidase, that can catalyze polymerization of monomers, preferably NIPAAm, and again following the stringent application of the secondary label the sample is subjected to the polymerization conditions of the respective methods and results in local formation of a polymer around the sequence of interest.

In alternate embodiments, polymerases direct the formation of polynucleotides within a local region of the nucleic acid, region or chromatin or chromosome using a mixture of nucleotides wherein one or more natural nucleotides is replaced or partially replaced with a nucleotide analogue containing a label precursor moiety. Label precursor moieties include but are not limited to moieties that can partake in click chemistry reactions, preferably SPAAC or CuAAC click chemistry reactions, nucleotides including but not limited to 5-DBCO-PEG4-dUTP, Azidomethyl-dUTP, Azide-PEG 4-aminoallyl-dUTP, 5-Ethynyl-dUTP, C8-Alkyne-dUTP, 5-trans-Cyclooctene-PEG4-dUTPS-Vinyl-dUTP, C8-Alkyne-dCTP, 5-DBCO-PEG4-dCTP, 5-Azido-PEG4-dCTP, N6-(6-Azido)hexyl-dATP. In some embodiments, polymerization proceeds via in situ PCR performed using one or more site-specific PCR primers, while in other embodiments nick translation or CRISPR-directed nick translation is performed, preferably to a region of repetitious sequence. The label precursor molecules are coupled to further molecules or labels, including but not limited to initiators for controlled polymerization as described above, preferably oxygen-tolerant ATRP, but alternately the label precursors are attached to pre-existing polymers via complementary reaction chemistries, preferably CuAAC click chemistry.

In some embodiments, a pair of labels are introduced as described above for polymerase or FISH based methods, the labels comprising one initiator label and one catalytic, photosensitizer or radical generation label as the sole source of radicals for a radical polymerization step using Activators ReGenerated by Electron Transfer (ARGET) Or Activators Generated by Electron Transfer (AGET) controlled radical polymerization.

In some embodiments, nucleic acids, chromatin or chromosomes are labeled using FISH-based or polymerization based methods as described above, and the labels are directly conjugated to oligonucleotide primer sequences, or conjugated to a polymer containing oligonucleotide primer sequences as practiced in the art of cluster-based DNA amplification for high throughput sequencing. The nucleic acids, chromatin or chromosomes are prepared into a sequencing library using amplification primers that are complementary to sequences present on the labels, such that the nucleic acids that amplify are those within the local vicinity of the attached oligonucleotide labels. The nucleic acids are subsequently removed and sequenced with next generation sequencing.

In some embodiments, staining is preceded by or contemporaneous with the addition of a matrix to the deposited sample on the fluidic device, the matrix possessing a density of strands sufficient to immobilize or restrict diffusion of the sample in the event that the sample is digested with nucleases or proteases. Non-limiting examples are the deposition of an agarose gel, preferably low-melting point agarose, acrylamide and methacrylate based gels including without limitation polyacrylamide and NIPAM, hydrogels, alginate gels, gelatin, gelatin crosslinked with crosslinkers including methacrylaltes, and gelatin crosslinked with reversible crosslinkers including without limitation Ditbiobis(succimidy propionate).

### Systems

In some embodiments of the device and method, the substrate comprising the surface onto which the chromosomes are positioned, has said molecules interrogated by an interrogation system. In some embodiments, the chromosomes are positioned on the surface of the substrate prior to bringing the substrate into contact with the interrogation system. In some embodiments, the chromosomes are positioned on the surface of the substrate while in contact with the interrogation system. For example, the interrogation system may comprise a device for blade coating the substrate with chromosomes. In some embodiments, at least one DNA molecule of the positioned chromosomes on the surface of the substrate is bound with at least one labelling body. In some embodiments, the binding of said labeling body is performed prior to contacting said substrate with the interrogation system. In some embodiments, the binding of said labeling body is performed while said substrate is in contact with the interrogation system. For example, the interrogation system may comprise a sub-system for exposing DNA chromosomes to a stain, and following rinsing and drying steps.

In some embodiments, the interrogation of the Chromosomes comprises fluorescent imaging. For example, the fluorescent imaging of a fluorescent physical map along an elongated molecule, where in some embodiments, said physical map is comprising a plurality of bound intercalating dyes varying in density per base-pair in correlation with the underlying AT-CG content to form a melt-map. In some embodiments, the optical interrogation of the chromosome comprises brightfield imaging. For example, the bright field imaging of a physical map within a metaphase chromosome, where in some embodiments, said physical map is comprising a plurality of bound stain dye molecules that vary in density within the chromosome in correlation with the local AT-CG content to form a karyotype banding pattern.

In embodiments whereby the chromosome is immobilized prior to optical or contact probe interrogation, the molecule may be modified to generate a physical map before or after immobilization.

In some embodiments, a subject computer program will store one or more of the following information: 1) the physical location(s) of an immobilized chromosome on a substrate or open fluidic device; 2) an in-silco representation of said molecule's physical map generated by interrogation, with said physical map mappable along or within said molecule in base-pair space, or with said physical map mappable to the physical coordinates along or within said molecule on the substrate or open fluidic devices in physical position space; and 3) any ROI(s) (Region of Interest(s)) along with their respective coordinates with respect to the originating molecule or underlying substrate or open fluidic device determined at least in part by an analysis of the physical map aligned to at least one reference.

In some embodiments, an ROI may be determined by the identification of a structural variation on a chromosome, or by the identification of a chromosome of unknown origin, or by the identification of a chromosome associated with a disease.

In some embodiments, the process for determination of the ROI(s) within a chromosome may include additional information from features obtained with the interrogation of said molecule. In some embodiments, the additional feature may include the identification of higher order structures in the molecule. In some embodiments, the additional feature may include the identification of knots, folds, loops, or spirals in the molecule. In some embodiments, the additional feature may include the identification of a chromosome being a circular molecule, or having originated from a circular molecule. In some embodiments, the additional feature may include the identification of at least one labelling body bound to the molecule. In some embodiments, the at least one labelling body bound to the molecule may associate said molecule with a type of chromosome, a disease, a structural variation, an originating cell type, including circulating tumor cell or circulating fetal cell. In some embodiments, the additional feature may include the identification of at least one protein bound to the molecule. In some embodiments, the additional feature may include the identification of variation in the molecule's stretch or density per unit length or area on the substrate or open fluidic device. As an example, an ROI may be selected based on the observation of a loop structure in the chromosome that is in proximity of a gene that is identified by an analysis of the physical map. In another example, an ROI may be selected within a certain region of the physical map of a chromosome coupled with the observation that the molecule is circular in topology. In another example, and ROI may be selected based on the observation of a protein bound to a chromosome that is in proximity of a transcription factor that is identified by an analysis of the physical map.

In some embodiments, the substrate or open fluidic device will include fiducials, or markers, or physical registration points that allow for the interrogation system to obtain a repeatable x-y coordinate grid of the surface of the substrate or open fluidic device.

In one preferred embodiment of the open fluidic device with chromosomes positioned on the surface, the input sample solution and any associated reagent solutions required to operate the device, can be loaded via manual pipette dispensing or automated liquid handling systems. In one preferred embodiment of the open fluidic device, the operation of the device may be controlled by at least one control instrument, which in turn, may be controlled by a program, computer based system, or a person(s). Operation of the device by the control instrument can include manipulating the physical position and conformation of the chromosome via the application of external forces, exposing the molecule to various reagent compositions or concentrations for various time periods or temperatures, optically interrogating the molecule to facilitate analysis of its composition or physical map as part of a feedback system to control the operation of the device, or extracting desired molecules or portions of molecules from the device. The open fluidic device and control instrument can interface in a number of ways. A Non exhaustive list includes: fluidic ports (both open and sealed), electrical terminals, optical windows, mechanical pads, heat pipes or sinks, inductance coils. A non-exhaustive list of potential functions the control instrument may perform on the device include: temperature monitoring, applying heat, removing heat, modifying an environmental condition, measuring an environmental condition, applying pressure or vacuum to ports, measuring vacuum, measuring pressure, applying a voltage, measuring a voltage, applying a current, measuring a current, applying electrical power, measuring electrical power, exposing the device to focused and/or unfocused light, collecting the light generated or reflected from the device.

In one embodiment, the operation of the interrogation of the chromosome on the substrate or open fluidic device is controlled by a control instrument. The control instrument may be centrally located, or have different parts distributed for different or redundant functions. In order to run the operation software on the control instrument, and perform collection and analysis of the data generated via interrogation, optically or with a contact probe, a non-exhaustive list of potential options include: localized processing within the control instrument, adjacent processing via a direct communication connection, external processing via a network connection, or combination there-of. Various examples of processing modules include: a PC, a micro-controller, an application specific integrated micro-chip (ASIC), a field-programmable gate array (FPGA), a CPU, a GPU, a network server, cloud computing service, or combinations there-of.

In some embodiments, the interrogation system may include an imaging system capable of optical interrogation, which may include any of the following types of imaging, or combinations there-of: fluorescent, epi-florescent, total internal reflection fluorescence, dark field, bright field, confocal.

In some embodiments, the interrogation system may be able to fire multiple light sources simultaneously, or in series, and be able to image multiple colors simultaneously, or in series. If imaging multiple colors simultaneously, this may be done on different cameras, on a single camera but different regions of the sensor array, or on the same sensor of the same camera. In some embodiments, the wavelength of light fired by the control instrument is chosen so as to interact with the sample, the sample labeling body, or a functionalized surface in some way. Non limiting examples include: photo-cleaving of the nucleic acid, photo-cleaving photo-cleavable linkers, manipulating optical tweezers, activating photo-activated reactions.

In some embodiments, the interrogation system may have at least one photosensitive sensor, of which non-limiting examples include: CMOS camera, SCMOS camera, CCD camera, photomultiplier tube (PMT), Time Delay & Integration (TDI) sensor, photodiode, light dependent resistor, photoconductive cell, photo-junction device, photo-voltaic cell.

In some embodiments, the interrogation system may have at least one xy-stage or xyz stage, allowing for the imaging system to image different regions of the device, or other devices in the control instrument.

In some embodiments, the interrogation system may have 1 or more motors or actuators capable of adjusting the device's interrogation region relative the interrogation system's optical path, including rotation, z, tip, and tilt, based on an auto-focus feedback system, software analysis of image quality, device accessibility requirements, user access, or combination there of.

The interrogation system may be capable of robotic transport of one or more devices to different parts of the control instrument.

In some embodiments the substrate or open fluidic device can include fiducial markers or alignment markers that can be used to enable visual alignment of the substrate or device either manually or with the control instrument's program. In some embodiments, there are multiple zones on the substrate or open fluidic device, with each zone designed to physically isolate different input samples. In some embodiments, there are fiducial markers on the substrate or device that guide the user or automated dispensing system where on the device to dispense solution.

In some embodiments where-by chromosomes can be transported in a fluidic channel of an open fluidic device, the molecule's physical map can be interrogated by flowing the molecule into the detection region for optical interrogation. In some embodiments where-by the chromosomes are immobilized on the surface of a substrate or open fluidic device, the immobilized molecules can be interrogated by physically moving the substrate or open fluidic device relative to the detection region for optical interrogation.

In some embodiments where-by the chromosome is transported through a fluidic channel of an open fluidic device, the interrogation system may interrogate the molecule's ROI while said ROI is contained within said fluidic channel's solution. In some embodiments, the interrogation system interrogates the molecule's ROI after at least the solution containing said ROI is removed, allowing for said ROI to be immobilized on the surface. In some embodiments, a solution may be re-introduced, allowing for re-suspension of the molecule within open fluidic device, and subsequent additional transport of the molecule via the application of an external force.

In some embodiments, the ROI may be selected at least in part from an analysis of the alignment of at least one molecule's physical map to at least one reference, with selection criteria that can change with time, including user preferences, the family health history of the originating sample's organism, the symptoms of the originating sample's organism, data from a clinical or biological or molecular test associated with the originating sample's organism. The ROI may be a gene, a structural variation (SV), a methylation pattern, a labelling body, a portion of a physical map, a sequence, a portion of a sequence, a higher order nucleic acid structure. The ROI may be an unidentified region within the physical map, or a region that may have an association with another ROL, directly or indirectly. The ROI may be a regulatory region, or a transcription factor binding site. The ROI may be associated with at least one disease. The ROI may be associated with risk factors for development or onset of at least one disease. The ROI may be a chromosomal region, a chromatin section, a compaction feature, an interaction or binding site, a regulatory factor or complex, a binding site, a transcription factor binding site, a TAD, a CRISPR binding site or complex, an SV, a phasing block, a regulatory or modification enzyme binding site, a restriction enzymes sequence motif, a methylation binding body, a centromeric region, a sub-telomeric region, a portion of telomere, a mobile element, a repetitive element, a viral insertion site. The ROI may comprise at least a portion of a higher order structure. The ROI may comprise at least one labelling body that is bound to the chromosome, or a bound to a body that is bound to the chromosome. The ROI may comprise a region within the chromosome where the desired genomic information is unclear or only partially known from the optical interrogation of the molecule's physical map, and for which a higher resolution interrogation is required For example, analysis of the physical map may suggest the presence of a series of repeats flanked between two known regions identified by comparison or alignment to a reference, however the repeated sequence is too small to allow for a precise count of the repeats to be determined by an analysis of the physical map generated by optical interrogation. In this example, targeted inspection of the repeat region by a contact probe may be used to elucidate a more accurate assessment of the number of repeats. The ROI may comprise a component for which there is a temporal or dynamic aspect that may change the nature of the ROI, for example a cohesin loop that is in the process of being extruded. The ROI may comprise a chromosome believed to have originated from a circulating tumor cell, or a cancer cell, or a circulating cancer cell, or a fetal cell, or a circulating fetal cell.

The ROI may be selected based on the positional relationship of various genomic information within the physical map with respect to each other. For example, an ROI may be selected based on the order in which certain genes are located with respect to each other within the physical map. The ROI may be selected based on the positional relation of a regulatory region and a gene with respect to each other in the physical map. The ROI may be selected based on the positional relationship of a various genomic information within the physical with respect to a labeling body or a higher order nucleic acid structure. For example, an ROI may be selected based on the physical proximity of a gene to a knot, or the physical proximity of a gene to a labeling body specifically bound to a promoter region.

The ROI may be selected at least in part by some computer algorithm, or patient diagnosis, or disease hypothesis, or experimental hypothesis. The ROI may be selected by the user on-the-fly, or selected based on observations and analysis of other ROIs. The ROI may be selected at least in part based on the analysis or alignment of physical maps of other chromosomes.

In some embodiments all identified ROI(s) are targeted. Alternately, not every or any ROI need be targeted. In some embodiments, ROI(s) are identified such that they inform the identification of additional ROI(s). In some embodiments, only a subset of ROI(s) are targeted. In some embodiments, a subset of ROI(s) from a first subset of molecules are used to identify an additional a subset of additional ROI(s) in a second subset of molecules. The first and second subsets of molecules can both each have an occupancy of at least one molecule, and the union of the first and second subsets can be zero or more molecules.

In the preferred embodiment, optical images of the surface of the substrate or open fluidic device are captured at a rate of more than 100 microns squared per second, or more than 1,000 microns squared per second, or more than 10,000 microns squared per second, or more than 100,000 microns squared per second, or more than 1,000,000 microns squared per second, or more than 10,000,000 microns squared per second. In some embodiments, adjacent images of the surface are stitched together to form a single optical image for analysis.

In the preferred embodiment, the more than 1 chromosome can be interrogated per second, or more than 10 chromosomes can be interrogated per second, or more than 100 chromosome can be interrogated per second, or more than 1,000 chromosome can be interrogated per second, or more than 10,000 chromosome can be interrogated per second.

In some embodiments, at least one ROI may be exposed to a solution, a reagent, a photon of a certain wavelength, or an environmental condition by the interrogation system.

In some embodiments, the interrogation system may comprise a dispensing system.

### Applications

In some embodiments the output of interrogation of the positioned chromosomes on the surface of a substrate comprises a karyotype. In some embodiments, each chromosome type within the karyotype is determined by a representative selection of chromosomes interrogated. For example, if 100 chromosome #1 from a human sample are interrogated, one such image from this set is selected as chromosome #1. In some embodiments, each chromosome type within the karyotype is determined by a selection criteria. For example, of all chromosome #1 interrogated, an image of the chromosome from this set with the desired banding density is selected, or the desired morphology. In some embodiments, each chromosome type within the karyotype is an insilico representation generated by an analysis of all the chromosomes interrogated that are of that particular type. For example, a representation of chromosome #1 is generated by averaging the images of all chromosome #1s identified. In some embodiments, a weighted averaging is performed, whereby the weighting scheme is a function of a desired property of each chromosome in the set. For example: banding density, morphology, or signal-to-noise ratio.

In some embodiments, the karyotype generated by the interrogation is used to diagnose chromosomal abnormalities. In some embodiments, these abnormalities may comprise structural variations. In some embodiments, these abnormalities may comprise Wolf-Hirschhorn syndrome, or Jacobsen syndrome, or Charcot-Marie-Tooth disease. In some embodiments, the abnormalities may comprise deletions, duplications, inversions, insertions, translocations, ring-formation, isochromosome-formation, and chromosome instability.

In some embodiments, the relative number of each chromosome type in the karyotype is determined by analyzing the relative number of each chromosome type identified by interrogation. For example, if 100 chromosome #22 were identified and 150 chromosome #21 were identified by interrogation, then the relative ratio of chromosome #22 to #21 could be interpreted as 2:3. In some embodiments, the actual sample chromosome type ratio and the identified chromosome type ratio may differ do to factors that can be compensated for during the calculation. For example: chromosome type selection bias from sample prep, or chromosome type selection bias from image recognition bias during interrogation. In some embodiments, the relative number of each chromosome type is used to diagnosis an aneuploidy. In some embodiments the aneuploidy may comprise a monosomy, or a trisomy, or a tetrasomy, or a pentasomy. In some embodiments, the aneuploidy may comprise Truner Syndrome, or Trisomy 21 (Down syndrome), or Trisomy 18, or Trisomy 13 (Edwards Syndrome/Patau syndrome), or sex chromosome trisomy.

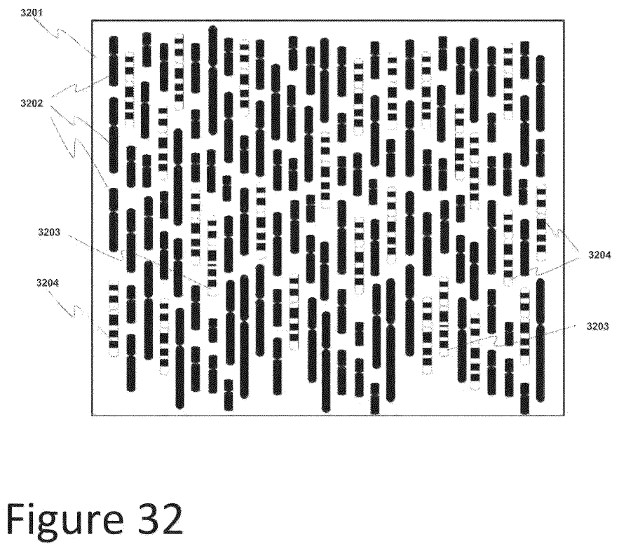

In some embodiments, the relative ratio of the number of “normal” to “abnormal” chromosomes of a certain type identified by interrogation is used to flag a rare event. This embodiment is demonstrated in FIG. 32 whereby chromosomes are positioned on a substrate (3201), and whereby there are 3 different chromosome sets identified by interrogation on the substrate. The first set of identified chromosomes in this demonstration are normal chromosome #10 (3204), recognized as such by their banding pattern. The second set of identified chromosomes in this demonstration are abnormal chromosome #10 (3203), recognized as such by their banding patterning showing an insertion. Finally, the third set of chromosomes (3202) are all other chromosomes that do not belong to the first or second set. In some embodiments, the ratio N:1 is sufficient to flag a rare event, whereby N is the number of normal chromosomes identified from a sample for every abnormal chromosome identified from a sample, and N is 1 or higher, or N is 10 or higher, or N is 100 or higher, or N is 1,000 or higher, or N is 10,000 or higher, or N is 100,000 or higher, or N is 1,000,000 or higher, or N is 10,000,000 or higher. In some embodiments, the rare event flag may be interpreted based on at least a certain condition of the patient from which the sample originated from. In some embodiments, the rare event flag may be interpreted as relative presence of cancer cells in the sample, or the relative number of fetal cells in the sample, or the relative number of abnormal cells in the sample.

In some embodiments, the “abnormal” chromosome is identified by a difference to a “normal” control chromosome. In some embodiments, the difference may comprise a genomic difference, a genomic variation, an epigenetic difference.

In some embodiments, the “abnormal” chromosomes may not be identifiable with respect to patient's “normal” chromosome, but instead may originate from a different organism from the patient, such as a bacteria or parasite.

In some embodiments, the rare event flag is used to identify early stage cancer, or the recurrence of cancer that previous was previously in remission, or to assess or monitor the progress of a cancer therapy, or to assess or monitor molecular residual disease (MRD) or long term presence of a pathogen or disease causing agent in an individual.

In some embodiments, the rare event flag is used to assess or monitor the genetic or epigenetic health of a patient, in particular, a patient who is at risk of random genomic or epigenetic aberrations from exposure to certain conditions, for example: chemical exposure, radiation exposure, extreme temperature exposure, toxin exposure, pollution exposure.

In some embodiments, an assessment or monitoring of the population of ecDNA in a sample is determined.

In some embodiments, nucleic acids, chromatin or chromosomes are probed using a plurality of labeling bodies sensitive to sequences, loci or chromatin domains per molecule such that multiple labeling bodies are present per chromosome, molecule, or macromolecule, and observation of multiple probes provides phasing linkages between the labeling body targets. In further embodiments, a plurality of chromosome types are each probed with a plurality of labeling bodies, the labeling bodies not necessarily being identical across different chromosomes. The labeling bodies include without limitation FISH probes, FISH probes to repeating regions, FISH probes to hypervariable regions, in situ PCR, single nucleotide polymorphism probes including without limitation labeled CAS-9 or catalytically inactive CAS-9, repeated CRISPR-CAS9 cutting followed by ligation, nick translation or other polymerase based incorporation of labelled nucleotides, most preferably fluorescently labeled nucleotides, restriction endonuclease based detection by nick translation or binding of labelled catalytically impaired restriction endonucleases, methyltransferase labeling, binding of labelled transcription factors, immunohistological or antibody-based recognition of DNA binding proteins including without limitation histones, transcription factors, enhancers, polymerases, cohesins, condensins, Ki-67 and enyzmes associated with DNA repair. In further embodiments, the same set of phasing probes are used to analyze parents and siblings to refine phasing data.

In some embodiments, non-invasive prenatal diagnosis (NIPT) is performed without enrichment for fetal cells, with cell types including lymphoid progenitors, hematopoietic stem cells, hematopoietic progenitor cells, mesenchymal stem cells, leukocytes, nucleated red blood cells and extravillous cytotrophoblasts.

In some embodiments, non-invasive prenatal diagnosis (NIPT) is performed with enrichment for fetal cells. Enrichment techniques are not limited to those described in Elicha Gussin, H. A. “Culture of Fetal Cells from Maternal Blood for Prenatal Diagnosis.” Human Reproduction Update 8, no. 6 (Nov. 1, 2002): 523-27 and Sabbatinelli, Giulia, Donatella Fantasia, Chiara Palka, Elisena Morizio, Melissa Alfonsi, and Giuseppe Calabrese. “Isolation and Enrichment of Circulating Fetal Cells for NIPD: An Overview.” Diagnostics 11, no. 12 (Nov. 30, 2021): 2239, and all references therein, including culture with low levels of erythropoietin, immunodepletion, immunoaffinity, fluorescence activated cell sorting, magnetic activated cell sorting, buoyant particle based cell sorting, dielectrophoretic particle separation, dielectrophoretic particle separation using immunolabeled particles, density gradient centrifugal separation, high molecular filtration, charge flow separation, lateral magnetophoresis, microfluidic methods, hyperaggregation, immunoaffinity chromatography, lectin induced separation, soybean agglutinin, selective lysis, selective lysis exploiting differential carbonic anhydrase activity and paramagnetic selection by the conversion of hemoglobin (Hb) into methemoglobin. Antibodies for recognition include antibodies with affinity for CD71, CD45, CD19, i-antigens, I-antigens, CD34, CD38, vimentin, fibronectin, vascular cell adhesion molecule, CD14, CD147, HLA antigens, zeta and epsilon chains of embryonic hemaglobin, gamma chain of fetal hemoglobin, 4B9, Gly A, CGB17, GB25, cytokeratins, CD105, human leukocyte G antigen, CD141.

In some embodiments, non-invasive prenatal diagnosis (NIPT) is performed in concert with metabolic labeling to differentiate maternal and fetal cells on the basis of Thymidine Kinase 1 levels. A thymidine analogue, preferably EdU, is added to cells prior to harvest, preferably a pulse of 1 hour followed by a 3 hr washout followed by 1 hour colcemid. Chromosomes are enriched and deposited on the surface of a fluidic device, preferably fixed, then stained with click chemistry and a labeling body, preferably a fluorophore, most preferably Cy5. Candidate fetal chromosomes are identified on the basis of enhanced labeling from the click conjugated label. In further embodiments, the thymidine analogue is introduced to cells that are not currently in S phase, including cells that are blocked with cell cycle inhibitors including nocodazole and cells that are in stationary phase or are exhibiting slow growth due to sub-optimal conditions not limited to crowding and depletion of nutrients in cell growth media. In further embodiments, the mixture of maternal and fetal cells are challenged with a low dose of ammonium chloride and potassium bicarbonate in the presence of an inhibitor of carbonic anhydrase II, acetazolamide. Only maternal cells contain carbonic anhydrase I, and will undergo osmotic stress, disrupting growth or lysing the cells (De Graaf, Irene M., Marja E. Jakobs, Nico J. Leschot, Ilya Ravkin, Simon Goldbard, and Jan M. N. Hoovers. “Enrichment, Identification and Analysis of Fetal Cells from Maternal Blood: Evaluation of a Prenatal Diagnosis System.” Prenatal Diagnosis 19, no. 7 (July 1999): 648-52). In this case the replication marker is added during or after the period of cell stress.

In some embodiments, non-invasive prenatal diagnosis (NIPT) is performed in concert with quantitative telomere labeling to differentiate maternal and fetal chromosomes, with fetal chromosomes displaying a greater length of telomeres.

In some embodiments, non-invasive prenatal diagnosis (NIPT) is performed in concert with FISH labeling to differentiate maternal and fetal content, whereby the FISH probe is selected for a gene known to be present or suspected to be present in fetal DNA such as Y chromosomal DNA in the case of a male fetus.

In some embodiments, non-invasive prenatal diagnosis (NIPT) is performed using a pair of maternal samples, one from before the pregnancy and one during the pregnancy. The samples are screened for structural and numerical aberrations and concerns are raised if the second sample contains aberrations not present in the first sample.

In some embodiments, at least one portion of one chromosome is isolated for analysis or further processing. In some embodiments, the processing comprises an isolation. In some embodiments, the processing comprises a reaction with a reagent. In some embodiments the reagent comprises an enzyme. In some embodiments the further processing comprises amplification. In some embodiments the further processing comprises sequencing. In some embodiments the further processing comprises capture into a droplet. In some embodiments, the further processing comprises exposure to a solution or gel dispensed from a dispensing system. In some embodiments, the further processing comprises further interrogation by a contact probe system, or physical manipulation by a contact probe system. In some embodiments, the further processing comprises exposure to at least environmental condition. In some embodiments, the chromosomes are positioned within the fluidic features of an open microfluidic device that includes patterning of topological and/or surface energy modifications to form wells on the surface of the device, so as to physically contain a dispensed solution within said wells.

Sequence Out of Place Analysis (SOPA): In some embodiments, chromosomes are deposited on a surface and probed for known sequences, preferably using a multiplicity of FISH probes, most preferably using sequential rounds of hybridization, clearing and rehybridization of a set of FISH probes, each with a different fluorescent label. In further embodiments, chromosomes are also analyzed to determine their identity by methods including without limitation morphological examination of chromosome shape, g-banding, arm length, centromere position, telomere position, in situ PCR and all other labeling and staining methods discussed herein. Interrogation results from the sequence FISH probing are analyzed by themselves or preferably analyzed as a collection of some or all FISH probes together, or most preferably combined with all FISH probe data and the interrogation results from chromosome identification to determine whether any sequences are present in unexpected locations, for example as a result of a chromosomal translocation.

In some embodiments, SOOPA is used to screen oncogenes to determine if oncogene relocation has occurred and to provide a means of screening against new, current, treated or relapsed cancer or to assess the degree of minimally resistant disease.

In some embodiments, a plurality of pachytene spermatocytes or oocytes are induced to the mitotic phase using, without limitation, Okadaic acid or Calyculin A and chromosomes are positioned on a fluidic substrate. Chromosomes are variously analyzed for aneuploidy, structural variations, epigenetic marks, viral integration events including HIV, telomere length, frequency with which two or more sequence variants, repeats, satellites, indels legions, other genomic feature, viral integration events including HIV appear on the same chromosome. In further applications spermatocyte screening is used to assess reproductive health of one or more human or non-human individuals, as a standalone sample of as a sequence exploring the effects of environmental perturbations on reproductive health, including without limitation exposure to chemicals, radiation, diet, food additives, microbiome, temperature, temperature of gonads and physiological exertion.

### Automated Interrogation

Target molecules arrayed on a surface are interrogated, for example via image capture. Target molecules having overlapping segments are optionally excluded from further analysis. Target molecules are then assessed through any number of approaches. Assessment may comprise grouping imaged target molecules as to similarity to one another or to expected target molecules, presence of a probe or label, local banding patterns or AT or GC content, arm length, arm number, centromere location, presence of any number of lesions, telomere length, or other characteristics.

The interrogation is often automated, such that target molecules are identified without requiring human involvement in the initial assessment process. Human oversight is in some cases used to confirm or assess the accuracy of automated assessment processes. However, in most cases the initial assessment is automated. This dramatically increases the throughput of some approaches, such that 10,000, 20,000, 50,000, 100,000, 200,000, 500,000, 1,000,000 or more target molecules can be rapidly assessed.

Assessment is effected through a computational moiety that is a structural component of a system, or can be effected remotely, through transmission of an interrogation outcome such a an image to a remote computation moiety. Assessment may be verified in full or in part by a human operator, such as a clinician trained in recognizing chromosomal aberrations.

Assessment outputs may variously comprise images, data related to chromosome abundance by type, relative frequencies, number and predicted identity of chromosomal lesions or pathogens.

### Definitions

As used herein “overlap” in the context of a nucleic acid or pair of nucleic acid segments refers a condition where, in a two dimensional view of a pair of nucleic acid segments, one segment cannot be viewed in total without a second segment being superimposed upon it.

“Chromosome” and “target molecule” are often used interchangeably in the disclosure herein. That is, methods, compositions and surfaces for the visualization or analysis of chromosomes may be used to accomplish the same for a broad range of target molecules, including wild type or structural variant human chromosomes, long nucleic acids, viral genomes, bacterial genomes or plasmids, mitochondrial genomes, or other potential targets for analysis herein.

A chromosome or other target molecule is “unmodified” if it does not comprise covalent bonds to exogenously added sequence as may be introduced through pcr amplification or other manipulation pursuant to sequencing library preparation.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The terms used in this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that way. Consequently, alternative language and synonyms may the same thing can typically be described in more than one be used for any one or more of the terms discussed here. Synonyms for certain terms are provided. However, a recital of one or more synonyms does not exclude the use of other synonyms, nor is any special significance to be placed upon whether or not a term is elaborated or discussed herein. All publications, patent applications, patents, and other references mentioned herein are incorporated by reference. In the case of conflict, the present specification, including definitions, will control. In addition, the materials, methods, and examples are illustrative only and are not intended to be limiting.

The invention is also described by means of particular examples. However, the use of such examples anywhere in the specification, including examples of any terms discussed herein, is illustrative only and in no way limits the scope and meaning of the invention or of any exemplified term. Likewise, the invention is not limited to any particular embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

As used herein, “about” or “approximately” in the context of a number shall refer to a range spanning +/−10% of the number, or in the context of a range shall refer to an extended range spanning from 10% below the lower limit of the listed range to 10% above the listed upper limit of the range.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

The use of the term “combination” is used to mean a selection of items from a collection, such that the order of selection does not matter, and the selection of a null set (none), is also a valid selection when explicitly stated. For example, the unique combinations including the null of the set {A, B} that can be selected are: null, A, B, A and B.

### Sample

The term “sample,” as used herein, generally refers to a biological sample of a subject which at least partially contains nucleic acid originating from said subject or patient. The biological sample may comprise any number of macromolecules, for example, cellular long nucleic acid molecules. The sample may comprise a cell or cell population. The sample may comprise a cell line or cell culture. The sample may comprise CTC (circulating tumor cells) or CFC (circulating fetal cells). The sample may comprise one or more droplets containing a biological material. The sample can include one or more microbes, virus, bacteria, pathogen, or parasite. The sample may comprise a nucleic acid. The biological sample may comprise material derived from another sample. The sample may comprise a tissue, such as a biopsy, core biopsy, needle aspirate, or fine needle aspirate. The sample may comprise a fluid, such as blood, a liquid biopsy, urine, or saliva. The sample may comprise skin. The sample may comprise a cheek swab. The sample may comprise plasma or serum. The sample may comprise a cell-free material such as extracellular polynucleotides. The sample may comprise material derived from blood, plasma, serum, urine, saliva, mucosal excretions, sputum, stool or tears, among others.

### Macromolecule

Used herein, “macromolecule” refers to a very large molecule composed of a thousand or more covalently bonded atoms. Macromolecules are often involved in biological processes, and are naturally formed. Examples include nucleic acid polymers, chromosomes, chromatin, long nucleic acid molecules, long nucleic acid molecules with a higher order structure, proteins, carbohydrates, or lipids. Non-biological macromolecules may comprise dendrimers, polymers, and carbon nanotubes, DNA origami.

### Nucleic Acid

The terms “nucleic acid”, “nucleic acid molecule”, “oligonucleotide” and “polynucleotide”, “nucleic acid polymer”, “nucleic acid fragment”, “polymer” are used interchangeably and refer to a polymeric form of nucleotides of any length, either deoxyribonucleotides or ribonucleotides, or analogs thereof, including both naturally occurring and synthesized. The terms encompass, e.g., DNA, RNA and modified forms thereof. Polynucleotides may have any three-dimensional structure, and may perform any function, known or unknown. Non-limiting examples of polynucleotides include a gene, a gene fragment, exons, introns, messenger RNAs (mRNA), transfer RNAs, ribosomal RNAs, IncRNAs (Long noncoding RNAs), lincRNAs (long intergenic noncoding RNAs), ribozymes, cDNA, ecDNAs (extrachromosomal DNAs) , Circular Polynucleotide, artificial minichromosomes, cfDNAs (circulating free DNAs), ctDNAs (circulating tumor DNAs), cffDNAs (cell free fetal DNAs), recombinant polynucleotides, branched polynucleotides, plasmids, vectors, isolated DNA of any sequence, control regions, isolated RNA of any sequence and configuration including circular RNA, nucleic acid probes, and primers.

Unless specifically stated otherwise, the nucleic acid molecule can be single stranded, double stranded, or a mixture there-of. For example, there may be hairpin turns or loops. Unless specifically stated otherwise, the nucleic acid molecule may contain nicks.

### Long Nucleic Acid Molecule

Unless specifically stated otherwise, a “long nucleic acid fragment” or “long nucleic acid molecule” is double strand nucleic acid of at least 100 kbp in length, and is thus a kind of macromolecule, and can span to an entire chromosome or multiple chromosomes. It can originate from any source, man-made or natural, including single cell, a population of cells, droplets, an amplification process, etc. It can include nucleic acids that have additional structure such as structural proteins histones, and thus includes chromatin. It can include nucleic acid that has additional bodies bound to it, for example labeling bodies, DNA binding proteins, RNA.

### Chromosome

A chromosome is a long nucleic acid molecule containing all, or part of the genetic material of an organism. In some embodiments, the chromosome comprises bound proteins. In some embodiments, the chromosome is a human chromosome. In some embodiments, the chromosome comprises at least one higher order nucleic acid structure. In some embodiments, the chromosome comprises a 3D structure. A chromosome may be an autosome or an allosome. A chromosome may be any type of chromosome (for example, any of the “normal” human chromosomes 1, 2, 3, 4, 5, 6, 7, 8, 9, 10, 11, 12, 13, 14, 15, 16, 17, 18, 19, 20, 21, 22, X, or Y, mitochondrial DNA), or combinations thereof, including at least a portion of at least one normal human chromosome type. A chromosome may comprise any state of the cell cycle, mitosis, and meiosis, including anaphase, metaphase, mitotic phase, prometaphase, prophase, synthesis phase, interphase, and resting phase. A chromosome in some cases refers to a nucleic acid to which chromosomal components are assembled, such as canonical huma chromosomes or ecDNA. A chromosome may be a plasmid, extrachromosomal DNA, mitochondrial DNA, eccDNA, ecDNA, a circular polynucleotide.

### Circular Polynucleotide

Used herein, “Circular Polynucleotide” or “Circular DNA” or “Circular Nucleic Acid” refers to a polynucleic acid molecule that forms a complete loop, and for which, is at least partially double-stranded. In some embodiments, the entirety of the molecule may double-stranded. In some embodiments, the molecule may have at least one nick, or at least one higher order nucleic acid structure, for example a hair pin.

The molecule may be a plasmid, a circular bacterial artificial chromosome, a circular yeast artificial chromosome, a circular Mitochondrial DNA, a circular Extrachromosomal circular DNA, a circular Chloroplast DNA, a circular chromosome, a circular virus, a covalently closed circular DNA. The molecule may comprise synthetic nucleotides, or natural nucleotides. The molecule may comprise synthetic sequence of nucleotides, wild sequence of nucleotides, cloned sequence of nucleotides, inserted sequence of nucleotides.

### Higher Order Nucleic Acid Structure

A “higher order nucleic acid structure”, or “structure”, or “higher order structure” refers to any 2nd, 3rd, or 4th order DNA structure, including any body bound to said nucleic acid molecule. The nucleic acid molecule may be linear or circular. Nucleic acids can have any of a variety of structural configurations, e.g., be single stranded, double stranded, triplex, replication loop or a combination of both, as well as having higher order intra-or intermolecular secondary/tertiary/quaternary structures, e.g., chromosomal territories, chromosome boundaries, chromosome regions, compartments, Topologically Associating Domains (TAD), chromatin loop and local direct regulatory factors binding, condensing associated loops, cohesin associated loops, guide nucleic acid, argonaut complexes, CRISPR Cas9 complexes, nucleoprotein complexes, insulator complexes, enhancer-promoter complexes, ribonucleic acid (RNA), small interfering RNA (siRNA), micro RNA (miRNA), guide RNA (gRNA), long non-coding RNA (IncRNA), repeat region binding proteins, telomere modification proteins, nucleic acid repair proteins, regulatory factor binding proteins, nucleic acid binding proteins, proteins, histone deacetylase (HDAC), chromatin remodeling protein, methyl-binding protein, transcription factor transcription complexes, bending with kinks of the genomic DNA polymers such as hairpins, replication loops, triple stranded regions, in cis or trans fashion etc. The nucleotides within the nucleic acid may have any combination of epigenomic state including but not limited to such as methylation or acetylation states. The nucleic acid can originate from any source, man-made or natural, including single cell, a population of cells, droplets, an amplification process, etc. In some embodiments, these structures include compounds and/or interactions of nucleic acids and proteins. In some embodiments, these structures include 2D and 3D configurations of the nucleic acid beyond the linear ID polymer chain. These 2D and 3D configurations can be formed via interactions with proteins, other nucleic acid molecules, or external boundary conditions. Non limiting examples of boundary conditions include a micro or nanofluidic chamber, a well on or in substrate or defined within a fluidic device, a droplet, a nucleus. The nucleic acid can include nucleic acids that has additional structure such as structural proteins including but not limited to such as any regulatory binding sites complexes, enhancer/transcription factor complex and their interaction with a nucleic acid molecule, Cohesins complex SMC (structural maintenance of chromosomes), ATPase subunits (Smc1 and Smc3), non-SMC regulatory subunits (Rad21/Scc1/Mcdand SA1/SA2/Scc3), Sgo1, mitotic kinases (pololike kinase 1 (Pik1) and aurora B), protein phosphatase 2A (PP2A), chromosome passenger complex (CPC), topo II decatenation, condesins, CTCF proteins, PDS5 proteins, WAPL proteins, condensin I, condensin II, CAP-G, histones and their derivative complexes, and thus includes chromatin. In some embodiments, higher order structure can include exogenous nuclei acid genome integration complex, in particular, an exogenous nuclei acid genome integration complex that comprises viral genome integration complexes or recombinant nucleus acid. In some embodiments, higher order structure can include extrachromosomal episomes physical docking complexes, in particular, where such complexes host chromosomes through binding sites. In some embodiments, the higher order nucleic acid structure comprises extrachromosomal nucleic acid deriving from a host chromosome. All of above, not limiting, could be target of labelling, physical or conformational biomarkers indicating the presence of certain state of genome organization or the shift between the states, that could be associated with pathogenomic consequences.

In particular, higher order nucleic acid structure can refer to the various levels of genome organization contained within a cell nucleus Jerković, Ivana & Cavalli, Giacomo. (2021). Understanding 3D genome organization by multidisciplinary methods. Nature Reviews Molecular Cell Biology. 22. 1-18, Kempfer, R., & Pombo, A. (2020). Methods for mapping 3D chromosome architecture. Nature Reviews Genetics, 21(4), 207-226 either individually, collectively, or a sub-set there of. Such genomic organization starts with linear primary DNA winding around histones to form nucleosomes, which are organized into clutches, each containing ~1-2 kb of DNA. Nucleosome clutches form chromatin nanodomains (CNDs) ~100 kb in size, where most enhancer-promoter (E-P) contacts take place. At the scale of~1 Mb, CNDs and CCCTC-binding factor (CTCF)-cohesin-dependent chromatin loops form topologically associating domains (TADs) and loop domains. On the higher scale up to 100s of megabases, chromatin segregates into gene-active and gene-inactive compartments (A and B, respectively) and into compartment-specific contact hubs, formation of sister chromatid axes. At the highest topological level, the nucleus is organized into chromosome territories.

Target molecule

The term “target molecule” encompasses any of the above molecule types or any other molecule to be assayed through the methods, compositions and systems of the disclosure herein.

### Hybridization

As used herein, the terms “hybridization”, “hybridizing,”hybridize,“annealing.” and “anneal” are used interchangeably in reference to the pairing of complementary or substantially complementary nucleic acids. Hybridization and the strength of hybridization (for example: the strength of the association between the nucleic acids) is influenced by such factors as the degree of complementary between the nucleic acids, stringency of the conditions involved, the Tm (melting temperature) of the formed hybrid, and environmental conditions, for example: temperature and pH. “Hybridization” methods involve the annealing of one nucleic acid to another, complementary nucleic acid, for example: a nucleic acid having a complementary nucleotide sequence.

Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In the context of this document, where hybridization occurs between nucleic acid strand and a double-stranded nucleic acid molecule, it should be understood that such hybridization is being done under conditions of either partial or full denaturation of the double-stranded nucleic acid molecule, unless otherwise specifically stated.

### Labeling Body

A “labelling body” or “label” used herein is a physical body that can bind to a nucleic acid molecule covalently or non-covalently, or to a body directly or indirectly bound to a nucleic acid molecule, which can be used to generate a signal that can be detected with interrogation, that differs from a detected signal (or lack there-of) that would be generated by said nucleic acid without said body. A series of non-limiting examples of labelling bodies follow: A labelling body may comprise a fluorescent intercalating dye that when bound to nucleic acid, can be used in a fluorescent imaging system to identify the presence of said nucleic acid. In another example, a labelling body may comprise a compound that binds specifically to methylated nucleotides, and gives a current blockade signal when transported through a nanopore, thus reporting a signal as to said molecule's methylation state. In another example, a labeling body may comprise a quantum dot. In another example, a labeling body may comprise a fluorescent probe specifically hybridized to a sequence of a nucleic acid, thus providing confirmation with a fluorescent imaging system that the sequence is present on said nucleic acid. In another example, a labeling body may comprise a fluorescent probe specifically binds to a specific protein (e.g.: DNA binding protein), with said protein bound to a long nucleic acid molecule or a macromolecule. In some cases, the absence of the labelling body, may comprise the signal. In some cases, the signal associated with the labelling body may comprise an attenuation, blocking, displacement, quenching, or modification of a signal from another labelling body. Non-limiting examples include: binding of a dark labelling body to the nucleic acid to displace an existing bond fluorescent body; binding of a dark labelling body to the nucleic acid to block a fluorescent labelling body from binding; quenching a near-by fluorescent labelling body bond to a nucleic acid; directly, or indirectly, reacting with a fluorescent labelling body bond to a nucleic acid to reduce its fluorescence. In some cases, the labelling body is not physically attached to the nucleic molecule at the time of interrogating said nucleic molecule and labelling body. For example, a labelling body may be attached to a nucleic acid molecule via a cleavable linker. At the desired time, the linker is cleaved, releasing said labelling molecule which is then detected by interrogation.

In some cases a labelling body comprises a multi-step labelling system, preferably with amplification at one or more steps, non-limiting examples including immunohistochemical detection with a primary antibody to the site of interest and a secondary antibody directed towards the primary antibody and attached to an addition labelling body.

In some cases the labelling body is capable of performing chemical reactions including without limitation photosensitizers, preferably Rose Bengal, radical generators, strong photoacids, chemical crosslinkers, moieties capable of click chemistry, strain-promoted Alkyne-Azide Click Chemistry (SPAAC) reactions including labels with Dibenzocyclooctyne moieties, but preferably CuAAC reactions, initiator sites for radical polymerization, most preferably initiator sites for controlled radical polymerization, most preferably 2-bromoisobutyrate groups for ATRP. In some cases labelling is comprised of two steps, the first step deposits labels and optionally washes excess label, while the second step combines further chemical reactants with the label to produce a further labelling body including without limitation the covalent or non-covalent attachment of light-absorbing. luminescent or fluorescent molecules, the production of light-absorbing, luminescent or fluorescent molecules from precursors, the growth of a polymeric matrix by growth of a polymer from a directly attached labelling body, with or without eventual attachment to molecules attached to the surface of fluidic device prior to deposition, the attachment of a pre-synthesized polymer or matrix to a directly attached labelling body, the growth of a polymeric matrix in the local vicinity of a labelling body where the growth is mediated by chemical diffusion in the local environment of the labelling body, and the continued growth of a polymeric matrix in the local vicinity of a labelling body where the growth is mediated by chemical diffusion in the local environment of the labelling body and an initial portion of the polymeric matrix was attached to the fluidic surface prior to deposition.

In some cases the labelling body is an enzyme capable of performing chemical reactions including without limitation alkaline phosphatase, horseradish peroxidase, beta-galactosidase, enzymes capable of light-generating reactions including without limitation bioluminescent proteins luciferase, firefly luciferase, click beetle luciferase, Renilla luciferase, NanoLuc, Gaussia luciferase, enzymes capable of removal of oxygen, preferably glucose oxidase, and enzymes capable of polymerization including oxidoreductases, peroxidases, peroxidases capable of polymerizing vinyl monomers, laccases, transferases, glycosyltransferases, phosphorylases, glycosyl transferases, acyltransferases, hydrolases, glycosidases, lipases, lipases capable of ring-opening polymerization of cyclic monomers, nucleases, endonucleases, exonucleases, RNAses and proteases.

### Barcodes

In some cases, a labelling body is comprised of a barcode consisting of polymers, peptide nucleic acids, polysaccharides, but preferably biological polymers including peptides and preferably nucleic acids, most preferably DNA. In some cases the barcode comprises a of positions wherein each position is deciphered independently and the identity of the barcode is determined by a combination of at least of portion of the positions. In some cases a nucleic acid barcode is attached using a polymerase to polymerize the barcode directly onto the sample as in the case of nick translation, or attached using a ligase, transposase, isomerase or other enzyme capable of creating phosphodiester bonds. In some cases the barcode is attached via non-enzymatic means, non-limiting examples being the use of SPAAC chemistry with metabolic labeling with 5-(Azidomethyl)-2′-deoxyuridine (AmdU) followed by reaction with a barcode or primer bearing an alkyne, alkyne derivative or preferably bicyclo[6.1.0]nonyne (BCN), or the use of CuAAC click chemistry to attach a barcode or primer bearing an azide or alkyne moiety, preferably attached to the 5′ end using a linker, to a complementary alkyne or azide attached to the nucleic acids, protein, chromatin or chromosomes under study, the attachments including metabolic labeling as in the case of EdU and crosslinking with crosslinkers comprising one moiety capable of click chemistry and another moiety capable of covalently or non-covalently binding the nucleic acids, chromatin or chromosomes under study. In some cases the labelling body is comprised of a scaffold structure including beads, high-molecular weight branched and unbranched polymers, nanoparticles, microparticles and viruses, one or more barcodes, present in one copy or preferably several thousand copies, most preferably in excess of several million copies, and in some cases functionalized surfaces or regions capable of binding to nucleic acids, chromatin or chromosomes, including but not limited to polymers containing a pl greater than the pH at which they are brought into contact with the sample, preferably amines, most preferably poly(ethyleneimine).

In some embodiments a barcode is a short nucleotide sequence (e.g., at least about 4, 6, 8, 10, 12, 14, 16, 18, 20, 25, 30, 35 nucleotides long) that encodes information. The barcodes can be one contiguous sequence or two or more noncontiguous sub-sequences. Barcodes can be used, e.g., to identify molecules in a partition or a bead, or a body to which an oligonucleotide is attached. In some embodiments, a bead-specific barcode is unique for that bead as compared to barcodes in oligonucleotides linked to other beads. In another example, a nucleic acid from each cell can be distinguished from nucleic acid of other cells due to the unique “cellular barcode.” Such partition-specific, cellular, or bead barcodes can be generated using a variety of methods. In some cases, the partition-specific, cellular, or particle barcode is generated using a split and mix (also referred to as split and pool) synthetic scheme, for example as described in Agresti, US2016/0060621. More than one type of barcodes can in some embodiments be in the oligonucleotides described herein.

In some embodiments, the information associated with the barcode may be an identification of a single, a particular, a type, a sub-set, a specific selection, a random selection, a group of body, where the body may be a molecule, a higher-order nucleic acid structure, an organelle, a sample, a subject. In some embodiments, the information associated with the barcode may be a process, a time-stamp, a location, a relationship with another body and/or barcode, an experiment id, a sample id, or an environmental condition. In some embodiments multiple information content may be stored in the barcode, using any encoding technique.

In some embodiments, the information encoded in the barcode includes uniquely identifying the molecule to which it is conjugated. These types of barcodes are sometimes referred to as “unique molecular identifiers” or “UMIs”. In still other examples, primers can be utilized that contain “partition-specific barcodes” unique to each partition, and “molecular barcodes” unique to each molecule. After barcoding, partitions can then be combined, and optionally amplified, while maintaining “virtual” partitioning based on the particular barcode. In some embodiments the presence or absence of a target nucleic acid comprising each barcode can be counted or tracked without the necessity of maintaining physical partitions.

In some embodiments, the barcode sequences are designed or randomly generated using a selection software for choosing barcodes that are: without hairpin, or containing even base composition (15%-30% A, T, G and C), or without homopolymers (default allows 3 bases of same nucleotides), or without simple repeats, or without low complexity sequences, or not identical to common vector or adaptor sequences. Furthermore, barcodes can be designed to be unique even if there are 3 mismatch sequencing errors.

Barcodes are typically synthesized and/or polymerized (e.g., amplified) using processes that are inherently inexact. Thus, barcodes that are meant to be uniform (e.g. a cellular, particle, or partition-specific barcode shared amongst all barcoded nucleic acid of a single partition, cell, or bead) can contain various N-1 deletions or other mutations from the canonical barcode sequence. Thus, barcodes that are referred to as “identical” or “substantially identical” copies can in some embodiments include barcodes that differ due to one or more errors in, e.g., synthesis, polymerization, or purification errors, and thus can contain various N-1 deletions or other mutations from the canonical barcode sequence. However, such minor variations from theoretically ideal barcodes do not interfere with the methods, compositions, and kits described herein. Therefore, as used herein, the term “unique” in the context of a particle, cellular, partition-specific, or molecular barcode encompasses various inadvertent N-1 deletions and mutations from the ideal barcode sequence. In some cases, issues due to the inexact nature of barcode synthesis, polymerization, and/or amplification, are overcome by oversampling of possible barcode sequences as compared to the number of barcode sequences to be distinguished (e.g., at least about 2-, 5-, 10-fold or more possible barcode sequences), or by using error correction encoding techniques. The use of barcode technology is well known in the art, see for example Shiroguchi, K., Jia, T. Z., Sims, P. A., & Xie, X. S. (2012). Digital RNA sequencing minimizes sequence-dependent bias and amplification noise with optimized single-molecule barcodes. Proceedings of the National Academy of Sciences of the United States of America, 109(4), 1347-1352 and [Smith, 2010]. Further methods and compositions for using barcode technology include those described in Agresti, 2016/0060621.

In some embodiments, at least a portion of the barcode can also be used as a primer binding site. In some embodiments, the primer binding site is for a PCR primer. In some embodiments, all barcodes that form a set of unique barcodes contain within said barcodes a globally identical primer binding site, such that a single primer sequence can be used to bind to all barcodes. In some embodiments, the primer will be the complement sequence of the primer binding site. In other embodiments, the primer will be the same sequence as the primer binding site, as the primer will bind to a previously amplified product of the original primer binding site. In some embodiments, there may be a combination.

In addition, in some embodiments, at least a portion of the barcode can also be used a primer.

### Interrogation

“Interrogation” is a process of assessing the state of a chromosome, a macromolecule, a nucleic acid, a long nucleic acid molecule, a higher order nucleic acid structure, a nucleic acid—protein complex, or other bio-molecule with an interrogation system. In some embodiments, the state of nucleic acid is assessed by interrogating the state of at least one labelling body on the nucleic acid by measuring a signal generated directly, or indirectly from the labelling body. It may be a binary assessment, such as the labelling body is present, or not. It may be quantitative, such as how many labelling bodies are present on a molecule. It may be a signal density or intensity along a line, an area, or volume. It may be a physical count, or distance between labelling bodies along the length the molecule.

In some embodiments, interrogation is used to generate a digitized or an in-silico representation of a physical map.

In some embodiments, interrogation is used to assess the physical state of a higher order nucleic acid structure. The physical state of the structure being interrogated may comprise the topology of the molecule such as the presence of a loop structure, a set of hierarchical loop structures, the number of supercoils present in a loop or the degree to which one or more loops from the same or separate molecules are intertwined. The physical state of the structure being interrogated may comprise the accessibility of a region of the nucleic acid to a binding partner or a cis or trans acting factor The physical state of the structure being interrogated may comprise the presence of partially replicated nucleic acid still in close proximity such as Okazaki fragments or a marker of newly synthesized nucleic acid such as results from a pulse of BrdU. The physical state of the structure being interrogated may comprise the level of cohesin left on metaphase chromosomes that has been manipulated experimentally or affected by genetic anomalies (e.g., by depleting either cohesin itself or Wapl), the resulting chromatids display substantially different lengths and shapes, becomes a quantitatively measurable biomarkers indicating of certain pathological states (Losada A, Hirano T. Dynamic molecular linkers of the genome: the first decade of SMC proteins. Genes Dev. 2005 Jun. 1; 19(11): 1269-87; Gandhi R, Gillespie PJ, Hirano T. Human Wapl is a cohesin-binding protein that promotes sister-chromatid resolution in mitotic prophase. Curr Biol. 2006 Dec. 19; 16(24): 2406-17; Shintomi K, Hirano T. Releasing cohesin from chromosome arms in early mitosis: opposing actions of Wap1-Pds5 and Sgo1. Genes Dev. 2009 Sep. 15; 23(18): 2224-36. The physical state of the structure being interrogated may comprise the amount, ratio, and distribution of condensins I and II in these chromatids. The physical state of structure being interrogated may comprise dynamic changes in genome organization, as in Cohesin release and sister chromatid resolution.

In some embodiments, the signal being interrogated may be fluorescent, photoluminescent, electro-magnetic, electrical, magnetic, physical, chemical, exhibit plasmon resonance or enhance raman signals by means of surface enhanced plasmon resonance.

The signal being interrogated may be analog or digital in nature. For example, the signal may be an analog density profile of the labelling body along the length of the nucleic acid in which the signal measured originates from multiple labelling bodies. In some embodiments, the state of the nucleic acid is directly interrogated without a labelling body, for example direct interrogation of a macromolecule or a long nucleic acid molecules in a cell via phase microscopy, or direct interrogation of a nucleic acid or macromolecule via a current blockade nanopore. Non exhaustive examples of different interrogation methods that may be used an interrogation systems either separately, or in combination include fluorescent imaging, bright-field imaging, dark-field imaging, phase contrast imaging, epi-florescent imaging, total internal reflection fluorescence imaging, nearfield/evanescent field imaging, a wave guide, a zero mode waveguide, plasmonic signaling, confocal, scattering, light sheet, structured illumination, stimulated emission depletion, super resolution, stochastic activation super resolution, stochastic binding super resolution, multiphoton, nanopore sensing of a current, voltage, power, capacitive, inductive, or reactive signal (either column blockade through the pore, and tunneling across the pore), chemical sensing (eg: via a reaction), physical sensing (eg. interaction with a sensing probe), SEM, TEM, STM, SPM, AFM. In addition, combinations of different labelling bodies and interrogation methods are also possible. For example: fluorescent imaging of an intercalating dye on a nucleic acid, while translocating said nucleic acid through a nanopore and measuring the pore current.

### Interrogation System

Used herein, “Interrogation System” is an automated, semi-automated, or manual system for interrogating the sample. In some embodiments, whereby the sample is interrogated while within or on a fluidic device, the interrogation system interfaces with the fluidic device and controls the operation of the fluid device. In some embodiments, the interrogation system comprises a multitude of separate systems that together can be coordinated by a controller or user. For example, an instrument for loading sample into a fluidic device, an instrument for flowing said sample in said fluidic device, an instrument for imaging said sample in said fluidic device, a controller for operating software for analysis of said imaging data. In some embodiments, the interrogation system comprises an integration of all or a sub-set of systems.

In some embodiments whereby a sample is contained within, or on, a fluidic device, operation of the device by the interrogation system can comprise: manipulating the physical position and conformation of the package, macromolecule, or long nucleic acid molecule via the application of external forces on said bodies; exposing the package, macromolecule, or long nucleic acid molecule to an environmental condition or reagent for a time period; optically interrogating the static or dynamic configuration of the package, macromolecule, or long nucleic acid molecule to facilitate analysis of their composition or as part of a feedback system to control operation of the device; extracting desired packages, macromolecules, or long nucleic acid molecules from the device. The fluidic device and interrogation system can interface in a number of ways. A non-exhaustive list includes: fluidic ports (both open and sealed), electrical terminals, optical windows, mechanical pads, heat pipes or sinks, inductance coils, fluid dispensing, surface scanning probes. A non-exhaustive list of potential functions the interrogation system may perform on the device include: temperature monitoring, applying heat, removing heat, applying pressure or vacuum to ports, measuring vacuum, measuring pressure, applying a voltage, measuring a voltage, applying a current, measuring a current, applying electrical power, measuring electrical power, exposing the device to focused and/or unfocused electromagnetic waves, collecting the electromagnetic waves light generated or reflected from the device, in far or near-fiend setting, creating and measuring a temperature, electromagnetic force, surface energy or chemical concentration differential or gradient, dispensing liquid into a device well or port, or on the device surface, contacting the device surface or entity on the device surface with a contact probe (for example: an AFM tip).

In some embodiments, confirmation of the presence of a long nucleic acid molecule, macromolecule, or package in a certain region of a fluid device and control over its physical position within said device is controlled by the interrogation system using a feedback controller system. Detection of the long nucleic acid molecule, macromolecule, or package is via detection of at least one interrogated signal. In the preferred embodiment, the signal is an electromagnetic signal originating from a labelling body bound to said long nucleic acid molecule, macromolecule, or package. In one embodiment, the control instrument feedback control system at least in part utilizes as input information the identification of a physical map profile within the long nucleic acid molecule, or absence of a physical map profile within the molecule.

In some embodiments, the interrogation system comprises localized computational processing modules within the system, adjacent computational processing modules via a direct communication connection, external computational processing modules via a network connection, or combination there-of. Various examples of computational processing modules include: a PC, a micro-controller, an application specific integrated micro-chip (ASIC), a field-programmable gate array (FPGA), a CPU, a GPU, System on Chip, a network server, cloud computing service, or combinations there-of.

The interrogation system may include at least one fluidic dispensing tip that is capable of dispensing fluid drops at the desired x, y, z coordinates on the surface of the device, and in some embodiments, extracting fluid drops at the desired x, y, z coordinates on the surface of the fluidic device. Fluid dispensing and extracting may be in volumes of microliters, nanoliters, picoliters, femtoliters, or attoliters.

The interrogation system may be able to illuminate multiple light sources simultaneously, or in series, and be able to image multiple colors simultaneously, or in series. If imaging multiple colors simultaneously, this may be done on different cameras, on a single camera but different regions of the sensor array, or on the same sensor of the same camera. In some embodiments, the wavelength of light illuminated by the control instrument is chosen so as to interact with the sample, the sample labelling body, or a functionalized surface in some way. Non limiting examples include: photo-cleaving of the nucleic acid, photo-cleaving photo-cleavable linkers, manipulating optical tweezers, activating photo-activated reactions, de-protecting photolabile protecting groups, IR thermal heating.

### Sequence

The term “sequence” or “nucleic acid sequence” or “oligonucleotide sequence” refers to a contiguous string of nucleotide bases and in particular contexts also refers to the particular placement of nucleotide bases in relation to each other as they appear in an oligonucleotide.

Sequencing can be performed by various systems currently available, such as, without limitation, a sequencing system by Illumina, Pacific Biosciences, Oxford Nanopore, Life Technologies (Ion Torrent), BGI, GenapSys, Element Biosciences, Singular Genomics Systems, Ultima Genomics. Sequencing can be performed by various technologies currently available, such as, without limitation: sanger sequencing, nanopore sequencing, nanogap sequencing, sequencing by ligation, sequencing by combinatorial probe anchor synthesis, sequencing by synthesis, pyrosequencing, polony sequencing, DNA nanoball sequencing, tunnelling current sequencing, sequencing by hybridization, sequencing by mass spectrometry, array-based sequencing, RNA polymerase based sequencing.

### Phasing

“Phasing” is the task or process of assigning genetic content to either the paternal or material chromosomes. The genetic content can be a nucleic acid molecule, a sequence, or a consensus from a set of sequences. The genetic content can be a single nucleic acid molecule whose sequence content may be known, unknown, or partially known. For example, it may be determined that a nucleic acid molecule originates from the mother, however the sequence content of said molecule is completely, or partially, unknown.

In some embodiments, within the context of this disclosure, phasing also refers to the identification that two separate genetic contents originate from the same maternal or paternal chromosome, however it may not be known to which, or that the two separate genetic contents originate from a different chromosome (one to the maternal, the other to the paternal), however again it may not be known to which. The said genomic content, in the concept of “genomic phasing” could be further expanded from separating the primary linear nucleic acid sequence information in the context of paternal, maternal, chromosomal, sister chromatids and extra-chromosomal entities, to include its native epigenomic information associated with the sequence, and to include the next level of secondary/tertiary/quaternary structures associated with the underlying sequence information, on maternal, paternal, chromosomal, sister-chromatids, large genomic regions and include but not limited to extra-chromosomal genomic entities, that were naturally occurring such as ecDNA or man-made artificial mini-chromosomes.

### Structural Variation

As used herein, “structural variation”, “structural variant”, or “SV” is the variation in structure of an organism's chromosome with respect to a genomic reference. These variations include a wide variety of different variant events, including insertions, deletions, duplications, retrotransposition, translocations, inversions short and long tandem repeats, rearrangements, and the like. These structural variations are of significant scientific interest, as they are believed to be associated with a range of diverse genetic diseases. In general, the operational range of structural variants includes events >50 bp, while the “large structural variations” typically denotes events >1,000 bp or more. The definition of structural variation does not imply anything about frequency or phenotypical effects.

### Reference

A “genomic reference” or “reference” is any genomic data set that can be compared to or aligned to another genomic data set. Any data formats may be employed, including but not limited to sequence data, karyotyping data, methylation data, genomic functional element data such as cis-regulatory element (CRE) map, primary level structural variant map data, higher order nucleic acid structure data, physical mapping data, genetic mapping data, optical mapping data, raw data, processed data, simulated data, signal profiles including those generated electronically or fluorescently. A genomic reference may include multiple data formats. A genomic reference may represent a consensus from multiple data sets, which may or may not originate from different data formats. The genomic reference may comprise a totality of genomic information of an organism or model, or a subset, or a representation. The reference may be a representation of a portion of a genome. The reference may be a representation of a portion of chromosome. The reference may be a representation of a gene or portion thereof. The reference may be a representation of a regulatory region or portion thereof. The reference may be a representation of a TAD, domain, region or portion thereof. The genomic reference may be an incomplete representation of the genomic information it is representing.

The genomic reference may be derived from a genome that is indicative of an absence of a disease or disorder state or that is indicative of a disease or disorder state. Moreover, the genomic reference (e.g., having lengths of longer than 100 bp, longer than 1 kb, longer than 100 kb, longer than 10 Mb, longer than 1000 Mb) may be characterized in one or more respects, with non-limiting examples that include determining the presence (or absence) of a particular feature, a particular haplotype, a particular genetic variations, a particular structural variation, a particular single nucleotide polymorphism (SNP), and combinations thereof, referring not only to being present or absent from the genomic reference in its entirety, but also from a particular region of genomic reference, as defined by the neighboring genomic content. Moreover, any suitable type and number of characteristics of the genomic reference can be used to characterize the sample nucleic acid, as derived (or not derived) from a nucleic acid indicative of the disorder or disease based upon whether or not it displays a similar character to the reference.

In some cases, the genomic reference is a physical map. This can be generated in any number of ways, including but not limited to: raw single molecule data, processed single molecule data, a digitized or an in-silico representation of a physical map generated from a sequence or simulation, a digitized or an in-silico representation of a physical map generated by assembling and/or averaging multiple single molecule physical maps, or combination there-of. For example, based on a known, or partially known sequence, a simulated in-silico physical map can be generated based on the method of generating a physical map used. In an embodiment where-by the physical map comprises labelling bodies at known sequences, a discrete ordered set of segment lengths in base-pairs can be generated. In an embodiment where-by the physical map comprises a continuous analog signal of labeling signal density along the sequence length, in base-pairs based on simulated local hydrogen bonds dissociation kinetics between the double helices, in chemical moiety modification, regulatory factor association or structural folding patterns based on nucleotide sequence and predicted functional element database maps.

In some cases, the genomic reference is data obtained from microarrays (for example: DNA microarrays, MMChips, Protein microarrays, Peptide microarrays, Tissue microarrays, etc), or karyotypes, or FISH analysis. In some cases, the genomic reference is data obtained from proximity 3D Mapping technologies or 3D physical mapping technologies.

In some cases, characterizations of the comparison or alignment with the genomic reference may be completed with the aid of a programmed computer processor. In some cases, such a programmed computer processor can be included in a computer control system.

### Alignment

An “alignment” is any process where-by genomic information that can be represented as a collection of information along at least one axis is statistically compared to at least one other genomic information that can be represented as a collection of information along at least one axis. In the preferred embodiment, the statistical comparison results in the orientation and overlap of the two genomic information that provides the best global similarity within their respective axis(axes). In the preferred embodiment, the statistical comparison output provides a similarity score or confidence score associated with the best global similarity, along with coordinates within their respective axis(axes) of the best global similarity. The genomic information can be raw, processed, digitized, in-silico, or simulated. Examples of different axis can include base-pairs, k-mers, domains, molecule length, molecule depth, molecule width, physical dimensions (for example: nm).

### Physical Mapping

“Physical mapping” or “mapping” of nucleic acid comprises a variety of methods of extracting genomic, epigenomic, functional, or structural information from a physical fragment of long nucleic acid molecule, in which the information extracted can be associated with a physical coordinate on the molecule. As a general rule, the information obtained is of a lower resolution than the actual underlying sequence information, but the two types of information are correlated (or anti-correlated) spatially within the molecule, and as such, the former often provides a ‘map’ for sequence content with respect to physical location along the nucleic acid. In some embodiments, the relationship between the map and the underlying sequence is direct, for example the map represents a density of AG content along the length of the molecule, or a frequency of a specific recognition sequence. In some embodiments, the relationship between the map the underlying sequence is indirect, for example the map represents the density of nucleic acid packed into structures with proteins, which in turn is at least partially a function of the underlying sequence. In some embodiments, the physical map is a linear physical map, in which the information extracted can be assigned along the length of an axis, for example, the AT/CG ratio along the major axis of long nucleic acid molecule. In some embodiments, the “linear physical map” or “ID physical map” is generated by interrogating labelling bodies that are bound along an elongated portion of a long nucleic acid molecule's major axis. For clarity, a string occupying 3D space in a coiled state can be represented as straight line, and thus extracted values along the 3D coil, can be represented as binned values along a ID representation of the string, and thus constitute a linear physical map. In some embodiments, the physical map is a “2D physical map”, in which the information extracted can be assigned within a plane that comprises the molecule, for example: karyotyping. In some embodiments, the physical map is a “3D physical map”, in which the information extracted can be assigned in 3D volume in which the molecule occupies. For example, tagging with super-resolution techniques to identify in (x, y, z) space the location of the tag within the chromosome as demonstrated with OligoFISSEQ Nguyen H Q, Chattoraj S, Castillo D, Nguyen S C, Nir G, Lioutas A, Hershberg E A, Martins N M C, Reginato P L, Hannan M, Beliveau B J, Church G M, Daugharthy E R, Marti-Renom M A, Wu C T. 3D mapping and accelerated super-resolution imaging of the human genome using in situ sequencing. Nat Methods. 2020 Aug; 17(8): 822-832, or in-situ genome sequencing, Payne A C, Chiang Z D, Reginato P L, Mangiameli S M, Murray E M, Yao C C, Markoulaki S, Earl A S, Labade A S, Jaenisch R, Church G M, Boyden E S, Buenrostro J D, Chen F. In situ genome sequencing resolves DNA sequence and structure in intact biological samples. Science. 2021 Feb. 26; 371(6532): eaay 3446.

In some embodiments, the physical map of a long nucleic acid molecule comprises multiple physical map types that are merged into a single physical map. For example, a long nucleic acid molecule with a fluorescent physical map that correlates with the localized AT density along the length of the molecule merged with a second physical map that indicates the locations of higher order structures along the length of the molecule.

The first and most widely used form of physical mapping is karyotyping, where-by metaphase chromosomes are treated with a stain process that preferentially binds to AT or CG regions, thus producing ‘bands’ that correlate with the underlying sequence as well as the structural and epigenomic patterns of the nucleic acid (Moore, C. M., & Best, R. G. (2001). Chromosome preparation banding. Encyclopedia of Life Sciences). However, the resolution of such a process with respect to nucleotide sequence is quite poor, about 5-10 Mbp, due to the condensed nature of nucleic acid being imaged. More recent methods of using linear mapping of elongated interphase genomic DNA have been generated by imaging nucleic acid digested at known restriction sites, Schwartz, 1988, U.S. Pat. No. 6,147,198, imaging attached fluorescent probes at nicking sites, Xiao, M., Phong, A., Ha, C., Chan, T. F., Cai, D., Leung, L., . . . Kwok, P. Y. (2007). Rapid DNA mapping by fluorescent single molecule detection. Nucleic Acids Research, 35(3), imaging the fluorescent signature of a nucleic acid molecule's methylation pattern, Sharim, H., Grunwald, A., Gabrieli, T., Michaeli, Y., Margalit, S., Torchinsky, D., . . . Ebenstein, Y. (2019). Long-read single-molecule maps of the functional methylome. Genome Research, 29(4), 646-656, imaging the fluorescent signature of a chromatin's histone, Lim S F, Karpusenko A, Sakon J J, Hook J A, Lamar T A, Riehn R. DNA methylation profiling in nanochannels. Biomicrofluidics. 2011 Sep; 5(3): 34106-341068. doi: 10.1063/1.3613671. Epub 2011 Jul. 25, electrical detection of bound probes to a nucleic acid through a sensor, Rose, 2013, US2014/0272954, and electrical detection of the methylation signature on a nucleic acid using a nanopore sensor, Rand, A. C., Jain, M., Eizenga, J. M., Musselman-Brown, A., Olsen, H. E., Akeson, M., & Paten, B. (2017). Mapping DNA methylation with high-throughput nanopore sequencing. Nature Methods, 14(4), 411-413.

Another method of linear physical mapping is to measure the AT/CG relative density or local melting temperature along the length of an elongated nucleic molecule. Such a signal can either be used to compare against other similar maps, or against a map generated in-silico from sequence data. There are many ways of generating such a signal. For example, the signal can be fluorescent or electrical in nature. Nucleic acid can be uniformly stained with an intercalating dye, and then partially melted resulting in the relative loss of dye in regions of rich AT content (Tegenfeldt, 2009, U.S. Pat. No. 10,434,512). Another method is to expose double stranded nucleic acid to two different species that compete to bind to the nucleic acid. One species is non-fluorescent and preferentially binds to AT rich regions, while the other species is fluorescent and has no such bias Nilsson, A. N., Emilsson, G., Nyberg, L. K., Noble, C., Stadler, L. S., Fritzsche, J., . . . Westerlund, F. (2014). Competitive binding-based optical DNA mapping for fast identification of bacteria-multi-ligand transfer matrix theory and experimental applications on Escherichia coli. Nucleic Acids Research, (1). Yet another method is to use two different color dyes that differentially label the AT and CG regions.

Mapping using such non-condensed interphase nucleic acid polymer strands bas improved upon the resolution of the primary sequence information, however the maps were stripped of any native structural folding or bound supporting proteins information and are often extracted from bulk solution of pooled samples with many potentially heterogeneous cells. Recently, 3D physical maps have been demonstrated where-by tags attached to chromosomes as specific locations are interrogated directly or indirectly to determine their relative position within the chromosome in 3D space (see Jerkovic, 2021 for a review of the various methods). These methods can include super resolution microscopy methods such as SIM, SMLM, and STED, Oligopaint FISH methods, multiplexed oligopaint FISH methods, and OligoFISSEQ methods. In addition, also included are in-situ sequencing methods such as OligoFISSEQ (Nguyen, 2020). Note, in this document, “3D physical Map(ping)” is different from “Proximity 3D Map(ping)”, which is defined elsewhere in this document.

### Genomic Information

The term “genomic information” or “genomic data” here includes any information content obtained directly or indirectly from the interrogation of a nucleic acid molecule that relates directly or indirectly to the underlying conventional genomic and epigenomic content of said molecule. Such information may include at least a portion of sequence information, a portion of a physical map, a banding pattern, an identification of chromosome number or origin. The information may include the orientation (5-prime, 3-prime) of the molecule with respect to said molecule's physical environment or the molecule itself, where the positional reference within the molecule may be physical length, sequence, a physical map along the molecule, or at least one labeling body bound to said molecule. The physical position of a base, or sequence with respect to said molecule's physical environment, or the molecule itself, where the positional reference within the molecule may be physical length, sequence, a physical map along the molecule, or at least one labeling body bound to said molecule. The physical position of a structural variant with respect to said molecule's physical environment, or the molecule itself, where the positional reference within the molecule may be physical length, sequence, a physical map along the molecule, or at least one labeling body bound to said molecule. The physical position of a higher order nucleic acid structure with respect to said molecule's physical environment, or the molecule itself, where the positional reference within the molecule may be physical length, sequence, a physical map along the molecule, or at least one labeling body bound to said molecule. The physical position of epigenetic data with respect to said molecule's physical environment, or the molecule itself, where the positional reference within the molecule may be physical length, sequence, a physical map along the molecule, or at least one labeling body bound to said molecule. The physical position of epigenetic data with respect to said molecule's physical environment, or the molecule itself, where the positional reference within the molecule may be physical length, sequence, a physical map along the molecule, or at least one labeling body bound to said molecule. The physical position of a labelling body bound to said molecule with respect to said molecule's physical environment, or the molecule itself, where the positional reference within the molecule may be physical length, sequence, a physical map along the molecule, or at least one additional labeling body bound to said molecule. The physical position of a body bound to said molecule with respect to said molecule's physical environment, or the molecule itself, where the positional reference within the molecule may be physical length, sequence, a physical map along the molecule, or at least one labeling body bound to said molecule.

### Binding

“Binding”, “bound”, “bind” as used herein generally refers to a covalent or non-covalent interaction between two entities (referred to herein as “binding partners”, e.g., a substrate and an enzyme or an antibody and an epitope). Any chemical binding between two or more bodies is a bond, including but not limited to: covalent bonding, sigma bonding, pi ponding, ionic bonding, dipolar bonding, metallic bonding, intermolecular bonding, hydrogen bonding, Van der Waals bonding. As “binding” is a general term, the following are all examples of types of binding: “hybridization”, hydrogen-binding, minor-groove-binding, major-groove-binding, click-binding, affinity-binding, specific and non-specific binding. Other examples include: Transcription-factor binding to nucleic acid, protein binding to nucleic acid.

### Specific Binding

As used herein, the terms “specifically binds” and “non-specifically binds” must be interpreted in the context for which these terms are used in the text. For example, a body may “specifically bind” to a nucleic acid molecule but have no significant preference or bias with respect the underlying sequence of said nucleic acid molecule over some genomic length scale and/or within some genomic region. As such, in the context of molecule's sequence, the body “non-specifically binds” to said nucleic acid molecule.

When in the context of binding between physically distinct molecules, “Specific binding” typically refers to interaction between two binding partners such that the binding partners bind to one another, but do not bind other molecules that may be present in the environment (e.g., in a biological sample, in tissue) at a significant or substantial level under a given set of conditions (e.g., physiological conditions).

### Substrate

As used herein, the term “substrate” is intended to mean a solid or semi-solid support that can serve as the foundation for a surface. In some embodiments, the substrate can comprise features. Non limiting examples of features include films, coatings, wells, immobilized molecules, pillars, channels, pits. The features can randomly positioned on the substrate, or patterned, or non-patterned, or uniform, or homogeneous, or inhomogeneous. A substrate as provided herein can be modified to accommodate attachment or immobilization of biopolymers by a variety of methods well known to those skilled in the art. Exemplary types of substrate materials include glass, modified glass, functionalized glass, inorganic glasses, silicon, silicon di oxide, silicon nitride, quartz, fused silica, microspheres, including inert and/or magnetic particles, polysaccharides, nitrocellulose, hydrogels, films, membranes, plastics (including e. g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

Those skilled in the art will know or understand that the composition and geometry of a substrate as provided herein can vary depending on the intended use and preferences of the user. Therefore, although planar substrates such as slides, chips or wafers are often exemplified herein, given the teachings and guidance provided herein, those skilled in the art will understand that a wide variety of other substrates exemplified herein or well known in the art also can be used in the methods and/or compositions herein.

The substrate may comprise of multiple substrates that are physically connected, for example using any combination of bonding mechanism, an adhesive, a film, a vacuum. The substrate may include various combinations of coatings. The substrate may have a patterned surface. The patterning may be additive or subtractive in nature, or combination of both. The substrate may comprise a component of a microfluidic device or a flow-cell. A substrate may be a film, which itself, may be in contact with another substrate.

A substrate can be of any desired shape. For example, a substrate can be typically a thin, flat shape (e.g., a square or a rectangle or oval). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

In some embodiments where the substrate is modified to contain one or more features, including but not limited to, wells, projections, ridges, features, or markings, the features can include physically altered sites. For example, a substrate modified with various features can include physical properties, including, but not limited to, physical configurations, magnetic or compressive forces, chemically functionalized sites, chemically altered sites, surface energy altered sites, hydrophobic/hydrophilic altered sites, and/or electrostatically altered sites.

In some embodiments, a substrate includes one or more markings on a surface of a substrate, e.g., to provide guidance for correlating spatial information with the characterization of interest. For example, a substrate can be marked with a grid of lines (e.g., to allow the size of objects seen under magnification to be easily estimated and/or to provide reference areas for counting objects). In some embodiments, fiducial markers can be included on a substrate. Such markings can be made using techniques including, but not limited to, printing, etching, sand-blasting, and depositing on the surface.

In some embodiments, a fiducial marker can be present on a substrate to provide orientation of the sample with features on the substrate, or the substrate itself.

In some embodiments, a substrate is also a fluidic device. In particular, an open fluidic device.

### Functionalized

A “functionalized” surface is a surface of a substrate that has been modified or engineered such as by certain chemicals, or macromolecules to elicit certain desired properties. For example: to bind specifically or non-specifically to a macromolecule, or to provide a reagent.

### Immobilized

As used herein, the term “immobilized” when used in reference to a molecule, a long nucleic acid molecule, a chromosome, a macromolecule, or body in direct or indirect attachment to a substrate via covalent or non-covalent bond(s) or stationery state by physical confinement, local energy minimization, local-entropy minimization, or held stationery by an external force. Indirect attached to the substrate may be via at least one additional intermediary molecule or body. In certain embodiments, covalent attachment can be used, but all that is required is that the molecules, long nucleic acid molecules, macromolecules, or bodies remain co-localized to the substrate under conditions in which it is intended to use. Non limiting examples include the entire molecule, long nucleic acid molecules, macromolecule, or body may be held stationary with respect to the substrate, or a portion of the molecule, long nucleic acid molecule, macromolecule, or body held stationary with respect to the substrate, while the remainder of the molecule, long nucleic acid molecule, macromolecule, or body has limited freedom of movement, or the molecule, long nucleic acid molecule, macromolecule, or body is indirectly attached to the substrate via an intermediary, and the entire molecule, long nucleic acid molecule, macromolecule, or body has some limited freedom of movement. For example, immobilization of an oligonucleotide to a substrate can occur via hybridization of said oligonucleotide to a secondary oligonucleotide, said secondary oligonucleotide at least partially containing a complementary sequence to the first, and itself immobilized to the substrate.

In certain embodiments, the molecule, long nucleic acid molecule, macromolecule, or body may be immobilized on a surface via a process that comprises physisorption, hydrogen bonding, van-der waals forces, or stiction.

In certain embodiments, the molecule or macromolecule can comprise biomolecules, nucleic acid molecules, proteins, peptides, nucleotides, long nucleic acid molecules, chromosomes, or any combination thereof. In certain embodiments, the body can comprise cells, droplets, dendrimers, packages, nucleosomes, beads, nanoballs, nanoparticles, nanodots, quantum dots.

Certain embodiments may make use of a substrate which has been functionalized, for example by application of a layer or coating of an intermediate material comprising reactive groups which permit covalent attachment to biomolecules, such as polynucleotides.

Exemplary bonding examples include click chemistry techniques, non-specific interactions (e.g. hydrogen bonding, ionic bonding, van der Waals interactions etc.) or specific interactions (e.g. affinity interactions, receptor-ligand interactions, antibody-epitope interactions, avidin-biotin interactions, streptavidin-biotin interactions, lectin-carbohydrate interactions, etc.). Exemplary bonding mechanism are set forth in U.S. Pat. No. Pieken, 1998, U.S. Pat. No. 6,737,236; Kozlov, 2003, U.S. Pat. No. 7,259,258; Sharpless, 2002, U.S. Pat. No. 7,375,234 and Pieken, 1998, U.S. Pat. No. 7,427,678; and US Pat Pub. No. Smith, US 2011/0059865, each of which is incorporated herein by reference.

### Surface Energy

Surface tension of a fluid is the energy parallel to the surface that opposes extending the surface. Surface tension and surface energy are often used interchangeably. Surface energy is defined here as the energy required to wet a surface. To achieve optimum wicking, wetting and spreading, the surface tension of a fluid is decreased and is less than the surface energy, of the surface to be wetted. The wicking movement of a fluid through the channels of a fluid device occurs via capillary flow. Capillary flow depends on cohesion forces between liquid molecules and forces of adhesion between liquid and walls of channel. The Young/Laplace Equation states that fluids will rise in a channel or column until the pressure differential between the weight of the fluid and the forces pushing it through channel are equal. Walter J. Moore, Physical Chemistry 3rd edition, Prentice-Hall, 1962, p. 730.

Δp=(2γ cos θ)/r

where Δp is the pressure differential across the surface, γ is the surface tension of the liquid, θ is the contact angle between the liquid and the walls of the channel and r is the radius of the cylinder. If the capillary rise is h and ρ is the density of the liquid then the weight of the liquid in the column is πr2ghρ or the force per unit area balancing the pressure difference is ghρ, therefore:

(2γcos θ)/r=ghρ

For maximum flow through capillary channels, the radius of the channel should be small, the contact angle θ should be small and γ the surface tension of the fluid should be large. The theoretical explanation of this phenomenon can be described by the classical model know as Young's Equation:

γSV=γSL+γLV cos θ

which describes the relationship between the contact angle θ and surface tension of liquid-vapor interface γLV, the surface tension of the solid-vapor interface γSV, and surface tension of the liquid-vapor interface γSL. When the contact angle θ between liquid and solid is zero or so close to 0, the liquid will spread over the solid. A contact angle measurement test is used as an objective and simple method to measure the comparative surface tensions of solids. In general, a material is considered to be hydrophilic when the contact angle in this test is below 90°. If the contact angle is above 90°, the material is considered to be hydrophobic.

### Combing

Defined herein, “molecular combining” or “combing” refers to the process of immobilizing at least a portion of a macromolecule, in particular nucleic acid molecules, to a substrate surface, or within a porous film on a substrate surface, such that at least a portion of the macromolecule is positioned on the surface of said substrate. The elongated portion can be fully immobilized to the substrate, or at least of portion of said portion have some degree of freedom. In some embodiments at least a portion of the molecule is elongated within a porous material film parallel to the surface of said substrate, or at least a portion of the molecule is elongated on top of a porous material film parallel to the surface of said substrate, or at least a portion of the molecule is elongated and suspended between two points. In some embodiments, the substrate surface is at least part of a fluidic device.

In one embodiment, a single nucleic acid molecule binds by one or both extremities (or regions proximal to one or both extremity) to a modified surface (e.g., silanised glass) and are then substantially uniformly stretched and aligned by a receding air/water interface. Schurra and Bensimon (2009) “Combing genomic DNA for structure and functional studies.” Methods Mol. Biol. 464: 71-90; See also U.S. Pat. No. Bensimon, 1995, U.S. Pat. No. 7, 122,647, both of which are herein incorporated by reference in their entirety.

The percentage of fully-stretched nucleic acid molecules depends on the length of the nucleic acid molecules and method used. Generally, the longer the nucleic acid molecules stretched on a surface, the easier it is to achieve a complete stretching. For example, according to Conti, et al. (2003) Current Protocols in Cytometry John Wiley & Sons, Inc, over 40% of a 10 kb chromosomes could be routinely stretched with some conditions of capillary flow, while only 20% of a 4 kb molecules could be fully stretched using the same conditions. For shorter nucleic acid fragments, the stretching quality can be improved with the stronger flow induced by dropping coverslips onto the slides. However, this approach may shear longer nucleic acid fragments into shorter pieces and is therefore may not suitable for stretching longer molecules. See e.g., Conti, et al. (2001) Current Protocols in Cytometry John Wiley & Sons, Inc. and Gueroui, et al. (Apr. 30, 2002) “Observation by fluorescence microscopy of transcription on single combed DNA.” PNAS 99(9): 6005-6010, both of which are hereby incorporated by reference in their entirety. See also Bensimon, 1994, U.S. Pat. No. 5,840,862, Bensimon, 1995, WO 97/18326, Bensimon, 1999, WO 00/73503, Bensimon, 1995, U.S. Pat. No. 7,122,647 which are hereby incorporated by reference in their entirety. Lebofsky, R., & Bensimon, A. (2003). Single DNA molecule analysis: Applications of molecular combing. Briefings in Functional Genomics and Proteomics, 1(4), 385-396, hereby incorporated by reference in its entirety.

In another embodiment, the nucleic acid molecule is stretched by dissolving the long nucleic acid molecules in a drop of buffer and running down the substrate. In a further embodiment, the long nucleic acid molecules are embedded in agarose, or other gel. The agarose comprising the nucleic acid is then melted and combed along the substrate.

In another embodiment, the nucleic acid molecule is combed on the surface by a receding meniscus, whereby the receding speed is controlled by a physical blade or mechanical fixture (herein collectively called “blade”) positioned above the surface onto which the molecule is to be combed, and said blade is moved relative to the surface of the combing surface, while maintaining a solution that comprises the meniscus pinned to the blade. In the preferred embodiment, the height of the blade and its speed relative to the combing surface are optimized for the combing application. In some embodiments, the blade's speed is more than 1 micron/second, or more than 10 microns/second, or more than 100 microns/second, or more than 1,000 microns/second. In some embodiments, the blade is in direct contact with the combing surface. In some embodiments, the blade is more than 1 micron above the combing surface, or more than 10 microns, or more than 100 microns, or more than 1,000 microns. In some embodiments, the height of the blade above the combing surface is maintained by a physical spacer. In some embodiments, the space is integrated in the blade. In some embodiments, the spacer is integrated in the substrate that comprises the combing surface.

In another embodiment, the nucleic acid molecule is combed on a transfer substrate, and then said transfer substrate is made contact with a target substrate, transferring the molecule. As an example, nucleic acid molecules are combed onto a PDMS substrate, which is then contacted with the target substrate, as previously demonstrated (Guan, Jingjiao, and L. James Lee. “Generating Highly Ordered DNA Nanostrand Arrays.” Proceedings of the National Academy of Sciences of the United States of America 102, no. 51(2005): 18321-25).

### Fluidic Device

The term “microfluidic device” or “fluidic device” as used herein generally refers to a device configured for fluid transport and/or transport of bodies through a fluid, and having a fluidic channel in which fluid can flow with at least one minimum dimension of no greater than about 100 microns. The minimum dimension can be any of length, width, height, radius, or cross-sectional axis. A microfluidic device can also include a plurality of fluidic channels. The dimension(s) of a given fluidic channel of a microfluidic device may vary depending, for example, on the particular configuration of the channel and/or channels and other features also included in the device.

A fluidic device may comprise any device that can contain milliliters volume (or less) of fluid. Physical boundaries that contain the fluid may be not necessarily be all solid, and can also include gas and/or secondary liquid boundaries. In some embodiments the microfluidic device can be exposed to a large volume of fluid during at least some portion of the operation, whereby the total volume exceeds milliliter scale. Non limiting examples include submerging the device in a reservoir, or submerging the device in a bath of liquid, or flowing large volumes of liquid through the device, or flowing large volumes of liquid over the device.

It should be understood that some of the principles and design features described herein can be scaled to larger devices and systems including devices and systems employing channels and features reaching the millimeter or even centimeter scale channel cross-sections. Thus, when describing some devices and systems as “microfluidic,” it is intended that the description apply equally, in certain embodiments, to some larger scale devices. In addition, it should be understood that some of the principles and design features described herein can be scaled to smaller devices and systems including devices and systems employing channels and features that are 100s of nanometers, or even 10s of nanometers, or even single nanometers in scale channel cross-sections. Thus, when describing some devices and systems as “microfluidic,” it is intended that the description apply equally, in certain embodiments, to some smaller scale devices. As an example, a device may have input wells to accommodate liquid loading from a pipette that are millimeters in diameter, which are in fluidic connection with channels that are centimeters in length, 100s of microns wide, and 100s of nm deep, which are then in fluidic connection with nanopore constriction devices that are 0.1-10 nm in diameter.

Microfluidic devices described herein can also include any additional components that can, for example, aid in regulating fluid flow, such as a fluid flow regulator (e.g., a pump, a source of pressure, etc.), features that aid in preventing clogging of fluidic channels (e.g., funnel features in channels; reservoirs positioned between channels, reservoirs that provide fluids to fluidic channels, etc.) and/or removing debris from fluid streams, such as, for example, filters. Moreover, microfluidic devices may be configured as a fluidic chip that includes one or more reservoirs that supply fluids to an arrangement of microfluidic channels and also includes one or more reservoirs that receive fluids that have passed through the microfluidic device. In addition, microfluidic devices may be constructed of any suitable material(s), including polymer species and glass, or channels and cavities formed by multi-phase immiscible medium encapsulation. Microfluidic devices can contain a number of microchannels, valves, pumps, reactors, mixers and other components for producing and manipulating (merging, sorting, splitting, etc.) the droplets. Microfluidic devices may contain active and/or passive sensors, electronic and/or magnetic devices, integrated optics, or functionalized surfaces. The physical substrates that define the microfluidic device channels can be solid or flexible, permeable or impermeable, or combinations there-of that can change with location and/or time. Microfluidic devices may be composed of materials that are at least partially transparent to at least one wavelength of light, and/or at least partially opaque to at least one wavelength of light.

A microfluidic device is typically designed and operated to manipulate a sample contained in a solution in order to achieve some desired outcome. The sample may be loaded into the device manually, for example using a pipette, or in an automated fashion, for example via an automated liquid handling system. The sample may further be introduced by exposing the microfluidic device to a fluidic stream. In addition, various other solutions including buffers and reagents may be added simultaneously with the sample input, or separately, at different input ports and/or different time points. In some cases, the microfluidic device is manufactured with liquids and reagents contained within.

A microfluidic device can be fully independent with all the necessary functionality to operate on the desired sample contained within. The operation may be completely passive, such as with the use of capillary pressure to manipulate fluid flows (Juncker, D., Schmid, H., Drechsler, U., Wolf, H., Wolf, M., Michel, B. Delamarche, E. (2002). Autonomous microfluidic capillary system. Analytical Chemistry, 74(24), 6139-6144, or may contain an internally power supply such as a battery. Alternatively, the fluidic device may operate with the assistance of an external device that can provide any combination of power, voltage, electrical current, electrical waveform, magnetic field, pressure, vacuum, light, heat, cooling, sensing, imaging, digital communications, encapsulation, acceleration forces, movement of one part of the microfluidic device relative to another, movement of a fluidic meniscus across at least a portion of the microfluidic device, sonic or ultrasonic energy, environmental conditions, etc. The fluidic device may also operate by chemical energy supplied by one or more fluids, solids or gasses applied to the device, such as energy from decomposition, combustion, chemical reaction of one more fluids solids or gasses, energy of mixing, hydration, solvation or desolvation of solids, fluids, and or gasses, wetting or meniscus forces, energy of phase transition from one state to another such as energy of vaporization or fusion. The external device maybe a mobile device such as a smart phone, a microcontroller based device, embedded computing platform, a larger instrument, with or without direct human control.

In some embodiments, the fluidic device includes an “electrowetting device” or “droplet microactuator”, which is a type of microfluidic device capable of controlled droplet operations within the fluidic device via specific application of local electric fields. Non limiting examples of such devices include a liquid droplet surrounded by air on an open surface, and a liquid droplet surrounded by oil sandwiched between two surfaces. A detailed review of the various configurations of use, and physics of droplet control are provided by Mugele, F., & Baret, J. C. (2005). Electrowetting: From basics to applications. Journal of Physics Condensed Matter, Vol. 17, pp. 705-774 and Zhao, Y.-P., & Wang, Y. (2013). Fundamentals and applications of electrowetting: A critical review. Reviews of Adhesion and Adhesives, 1(1), 114-174, both of which are provided here for reference.

Unless specifically stated otherwise, a “fluidic feature” is a feature within or on the fluidic device with a property that comprises the ability by itself, or conjunction with other fluidic features, to at least partially contain, directly or indirectly, a liquid or body within said fluidic feature(s), or a property that comprises the ability to interact with a body. Examples of bodies include macromolecules, cells, nucleus, nucleosomes, microconidia, DNA, chromosomes, ecDNA, circular polynucleotides, proteins, enzymes, nucleotides, carbohydrates, RNA, beads, dendrimers, nanoparticles, lipids, nanoballs, quantum dots, Pickering emulsion, and droplets.

In some embodiments, the fluidic feature's interaction with the with a biological body may comprise: shear-force, or bonding, or repulsion, or attraction, puncture, compression, extrusion, expansion, electrochemical reaction, or charge exchange. In some embodiments, the fluidic feature comprises a physical obstacle.

In some embodiments the fluidic feature may comprise a channel, a wall, a corner, an edge, a barrier, a physical obstacle, a pillar, a pit, a cone, a rod, a sphere, a partial sphere, an oval, a circle, a polygon, a cylinder, a mound, a bill, a trough, a cube, a parallelogram, an ellipse, a triangle, a trapezoid, a trapezium, a crescent, a cuboid, an ellipsoid, a triangular prism, a pyramid, a tetrahedron, an octahedron, a line, a dash, a dot, a torus, a prism, a polyhedron.

In some embodiments, the fluidic feature may comprise a region with a desired surface energy property, or desired surface hydrophobicity property, or desired surface hydrophilicity property, or desired anti-fouling property, or desired surface-roughness property, or desired surface-smoothness property.

In some embodiments, an antifouling property or behavior includes preventing the adhesion of an intact package, semi-intact package, long nucleic acid molecule, macromolecule, or molecule to at least a portion of the surface with said property. In some embodiments, the antifouling property is further influenced by the chemical composition of the delivery fluid or at least one environmental condition. The optimum antifouling surface treatments for the various classes of intact packages, semi-intact packages, long nucleic acid molecule, macromolecules, and molecules are not necessarily the same, and the effects of a particular antifouling coating can be influenced by prior treatment of the package, long nucleic acid molecule, macromolecule, or molecule.

In some embodiments, a surface roughness property is correlated with the preventing, or promoting of the adhesion of an intact package, a semi-intact package, a long nucleic acid molecule, a macromolecule, or a molecule to at least a portion of the surface with said property. In some embodiments the roughness is more than 0.1 nm rms, or more than 0.2 nm rms, or more than 1 nm rms, or more than 2 nm rms, or more than 5 nm rms, or more than 10 nm rms, or more than 20 nm rms, or more than 50 nm rms, or more than 100 rms, or more than 200 nm rms, or more than 500 rms, or more than 1000 nm rms. In some embodiments the surface roughness is selected to achieve a certain hydrophobic property. In some embodiments, the surface roughness is selected to achieve a certain hydrophilic property. In some embodiments, the surface roughness is selected to achieve a certain anti-fouling property. In some embodiments, the surface roughness is tuned to achieve a certain adhesion property.

The containment or flow of a fluid or body “within the fluidic feature(s)” of the fluidic device can be by any means in which at least a portion of the fluid or body can be maintained within, among, along, or on, at least a portion of at least one fluidic feature defined within or on the fluidic device for a period of time.

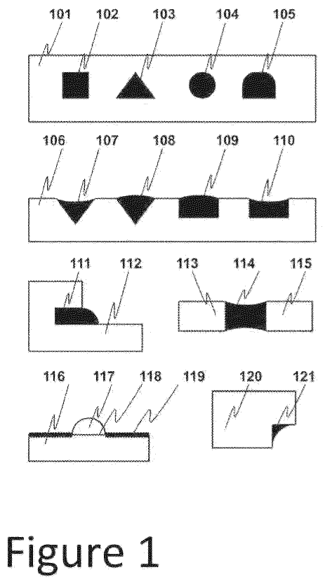

In some embodiments, at least a portion of the fluid is contained by the solid or semi-solid physical boundaries of the channel walls. FIG. 1 shows embodiments where-by channel walls with cross-sections such as rectangles (102), triangles (103), ovals (104), and mixed geometry (105) are all defined within a fluidic device (101). In other embodiments, fluidic containment within the fluidic device may be at least partially contained via solid physical features in combination with surface energy features Casavant, B. P., Berthier, E., Theberge, A. B., Berthier, J., Montanez-Sauri, S. I., Bischel, L. L., . . . Beebe, D. J. (2013). Suspended microfluidics. Proceedings of the National Academy of Sciences of the United States of America, 110(25), 10111-10116, or an immiscible fluid Li, C., Hite, Z., Warrick, J. W., Li, J., Geller, S. H., Trantow, V. G., . . . Beebe, D. J. (2020). Under oil open-channel microfluidics empowered by exclusive liquid repellency, Sci adv 2020 Apr. 17; 6(16)eaay9919, etrieved from website://advances.sciencemag.org/. Examples of a fluid being at least partially confined within physical boundaries include various channels physically defined on the surface of a fluidic device (106) such as grooves (107, 108) and rectangles (109, 110), all of which can contain at least a portion of a fluid via surface tension. In other embodiments, the channel (111) could be a defined by a groove in a corner (112) of a fluidic device, or the channel (114) could be defined by two physically separated boundaries (113 and 115) of a fluidic device, or the channel (121) could be defined by a corner (120) of a fluidic device. In other embodiments, the channel (217) is defined by a hydrophilic section (218) on the surface of a fluidic device (216) where-by the hydrophilic section is bounded by hydrophobic sections (219) on the surface of the fluidic device. In all cases, these embodiments are non-limiting examples and additional embodiments include combinations thereof. Used herein, an “open fluidic device” is a fluidic device that comprises at least one fluidic feature (for example: a channel) in which the solution in said fluidic feature is at least partially exposed to a gas-phase interface. Examples include air, water vapor, solvent vapor, oxygen, nitrogen, or mixtures thereof. In particular, with regards to the operation of an open fluidic device, the selection of the gas composition, pressure, and other environmental conditions may be controlled, and may be critical to the desired operation of the open fluidic device. For example, for a particular period of time, a particular temperature, or humidity, or dew point, or solvent vapor partial pressure, or wavelength exposure may be desired.

A multitude of fluidic features with desired surface property regions are possible. Non-limiting examples of surface properties include a degree of hydrophobicity, degree of hydrophilicity, or degree of anti-fouling, or degree of roughness, or degree of smoothness, or surface charge density, or surface functionalization, or surface energy. In some embodiments, a fluidic feature may comprise one region with a desired surface property. In some embodiments, a fluidic feature may comprise at least one region with a desired surface property. In some embodiments, a fluidic feature may comprise a multitude of regions, each with a specific desired surface property.

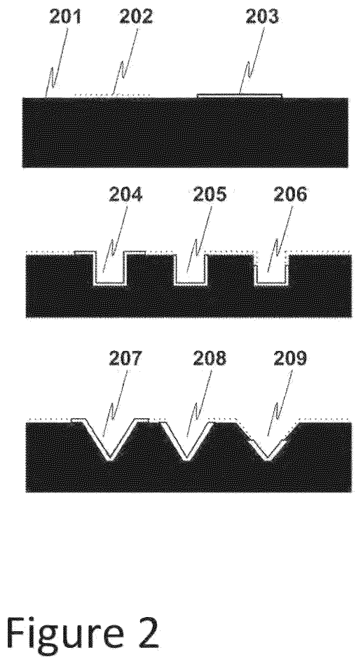

FIG. 2 demonstrates some embodiments of fluidic features with selective patterning of different desired surface properties of type “A” (202) and type “B” (203) on the surface of a fluidic device (201). The fluidic features defined on the fluidic device may take on any shape. In addition, any surface region on any said shape, including the entirety or portion of a surface plane, may comprise a desired surface property. For example, fluidic features comprising of open fluidic rectangular cross-section channels (204, 205, 206) may have different regions with a surface property “B” inside the channel, and surface property “A” outside the channel. In another example, fluidic features comprising of open fluidic triangular cross-section channels (207, 208, 209) may have different regions with a surface property “B” inside the channel, and surface property “A” outside the channel.

There are numerous different methods for modifying the surface energy properties or anti-fouling properties of a surface. For example, by contact transfer stamping of a hydrophobic or anti-fouling molecule to the glass fluidic device surface, leaving the naturally hydrophilic open and depressed channels on the fluidic device surface un-modified. Other methods may comprise spin coating a film, dip coating a film, growing a film, depositing a film, printing a film, dispensing a film, spraying a film, self-assembling a monolayer. Modification of the surface may be selective to certain regions, wherein the modified regions are selected by the means of modification, or their physical position relative to other regions, or use of a mask, or use of a temporary sacrificial material. Modification of the surface may be selective to certain regions, wherein the modified regions are selected by the means of chemistry, such that only materials of certain surface chemistry properties will participate in the modification process. Modification of the surface may be selective to certain regions that comprise a seed, or additional material that comprises a surface property that allows for selective modification.

Transfer contact printing (or micro-contact printing) is a widely used method of transferring a molecule from a donor substrate to an acceptor substrate. The most common donor substrates are typically flexible elastomers that provide some degree of compliance with the acceptor substrate. For a review of different micro-contact methods and materials refer to Rachel K Smith, Penelope A Lewis, Paul S Weiss, Patterning self-assembled monolayers, Progress in Surface Science, Volume 75, Issues 1-2, 2004, Pages 1-68, Lamping, Sebastian, Christoph Buten, and Bart Jan Ravoo. “Functionalization and Patterning of Self-Assembled Monolayers and Polymer Brushes Using Microcontact Chemistry.” Accounts of Chemical Research 52, no. 5 (May 21, 2019): 1336-46, and Qiu, Shi, Jiawen Ji, Wei Sun, Jia Pei, Jian He, Yang Li, Jiao Jiao Li, and Guocheng Wang. “Recent Advances in Surface Manipulation Using Micro-Contact Printing for Biomedical Applications.” Smart Materials in Medicine 2 (2021): 65-73. In some embodiments the transferred molecules form a monoloyer on the acceptor substrate, or a bilayer, or a multi-layer, or a concatemer, or a film. In some embodiments, multiple contact printing steps may be employed.

In some embodiments the surface modifications modulate the hydrophobicity of a portion of the fluidic device, and work in concert with topological features to direct fluidic flow, specifically the flow of an interface that separates phases, most preferably the meniscus between a fluid and a gas phase. Surface modifications with a high degree of hydrophobicity are not limited to the class of molecules with one or more pendant halogenated chains such as poly(heptadecafluorodecyl acrylate), Octadecyltrichlorosilane or 1H, 1H,2H,2H-Perfluorodecylamine. Surface modifications with a high degree of hydrophobicity are not limited to the class of molecules with aliphatic groups such as lauryl methacrylate, Octadecyltrimethoxysilane, or Dodecylamine.

In some embodiments, the hydrophobicity of the surface modifications applied to a portion of the device can be altered by mild reaction or environmental conditions. The adaptable hydrophobicity surface modifications are not limited to polymers, present as homopolymers, copolymers or block-co-polymers, made from labile or cleavable groups that include (Trimethylsilyl)methacrylate, t-BOC protected amines such as N-(3-BOC-aminopropyl)methacrylamide, 3-[(2-Aminoethyl)dithio]propionic acid, enzymatically cleavable phenylalanine rich peptides conjugated to polyacrylic acid, and polymers decorated with terminal nitrobenzyl groups that are further conjugated to fluorinated alkanes. The adaptable hydrophobicity surface modifications are not limited to the group of thermoresponsive polymers that includes cellulose nanocrystals, Poly(N-isopropylacrylamide), Pluronic F127, poly-(organophosphazene), and copolymers of the above including poly(N-isopropylacrylamide) copolymerized with poly(ethylene glycol).

In some embodiments, the surface modifications add a chemical moiety that interacts with a portion of the package or with a portion of the macromolecules or with a portion of the long nucleic acid molecule. Further, surface modifications can be combined with one or more of the above strategies for dynamic modification of the surface modifications. The surface modifications are not limited to polymers and molecules containing moieties that are multivalent, including linear and branched polyamines and including spermidine and spermine moieties, peptides rich in charged amino acids including lysine, glutamic acid, arginine, histidine and aspartic acid, oligonucleotides, homopolymers of charged amino acids, copolymers, graft polymers, and co-block polymers of charged amino acids, polyacrylic acid, poly(methacryol-1-lysine), poly(ethyleneimine), poly(2-Aminoethyl methacrylate), poly(ethylene glycol), poly(N-[3-(N,N-Dimethylamino)propyl] acrylamide), poly(2~(tert-Butylamino)ethyl methacrylate), poly(2-Diisopropylaminoethyl methacrylate).

The surface modifications include and are not limited to molecules, polymers and macromolecules that react to form covalent bonds with packages and macromolecules, as homopolymers, copolymers or co-block polymers, either self-reactive or reactive in combination with chemical crosslinkers such as formaldehyde, paraformaldehyde and EDC/NHS, the group including Poly(ethylene glycol) diamine, Cinnamyl methacrylate, poly(glycidyl methacrylate), poly(propargyl methacrylate), poly(2-Aminoethyl methacrylate).

The surface modifications include and are not limited to molecules, polymers and macromolecules that interact specifically with the membranes, lipid bilayers or other such components that form the envelope of packages or interact with the meniscus separating multiple fluidic or gaseous phases. These surface modifications, whether as homopolymers, copolymers, graft polymers, or co-block polymers, are not limited to pendant phospholipids and aromatic groups added to the end of a polymer including 1-decyne added to a NaN3 substituted ATRP polymer end group via a Cu catalyzed cycloaddition.

In some embodiments, the modification is a “grafting to” reaction that adds a small molecule, polymer or macromolecule to at least a portion of the fluidic device or a fluidic feature. Some embodiments utilize the reaction of silanes to glass, silicon or oxide surfaces, the silanes including but not limited to aminopropyl (3-Aminopropyl)triethoxysilane, mPEGSK-Silane and 3-(Trimethoxysilyl)propyl acrylate. In some embodiments, non-covalent interactions are formed with surface modifying chemicals including poly-1-lysine, most preferably with co-block polymers including the family of Pluoronic detergents, Tween 20 and Tween 80 and others described in this document.

A variety of materials and methods, according to certain aspects of the invention, can be used to form articles or components such as those described herein, e.g., channels such as microfluidic channels, chambers, etc. For example, various articles of components can be formed from solid materials, in which the channels can be formed via micromachining, film deposition processes such as spin coating and chemical vapor deposition, laser fabrication, photolithographic techniques, bonding techniques, deposition techniques, lamination techniques, molding techniques, imprinting techniques, 3D printing techniques, Fused deposition modeling 3D printing, stereolithography 3D printing, selective laser sintering 3D printing, etching methods including wet chemical or plasma processes, multi-phase immiscible medium encapsulation and the like. For patterning, a variety of methods may be employed, including but not limited to: photolithography, electron-beam lithography, nanoimprint lithography, AFM lithography, STM lithography, laser writer, focused ion-beam lithography, stamping, embossing, molding, and dip pen lithography. For bonding, a variety of methods may be employed, including but not limited to: thermal bonding, adhesive bonding, pressure bonding, vacuum bonding, surface activated bonding, fusion bonding, anodic bonding, plasma activated bonding, laser bonding, and ultra sonic bonding.

In one set of embodiments, various fluidic features, structures, or components of the articles described herein can comprise of a polymer, for example, an elastomeric polymer such as polydimethylsiloxane (“PDMS”), polytetrafluoroethylene (“PTFE” or Teflon®), or the like. For instance, according to one embodiment, a microfluidic channel may be implemented by fabricating the fluidic system separately using PDMS or other soft lithography techniques, “Soft Lithography,” by Younan Xia and George M. Whitesides, Annual Review of Material Science, 1998, Vol. 28, pages 153-184; “Soft Lithography in Biology and Biochemistry,” by George M. Whitesides, Emanuele Ostuni, Shuichi Takayama, Xingyu Jiang and Donald E. Ingber, Annual Review of Biomedical Engineering, 2001, Vol. 3, pages 335-373.

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), cyclo-olefin polymer (COP), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, nylon, or the like. Combinations, copolymers, or blends involving polymers including those described above are also envisioned.

In some embodiments, various structures or components of the article are fabricated from polymeric and/or flexible and/or elastomeric materials, and can be conveniently formed of a phase changing fluid, facilitating fabrication via molding (e.g. replica molding, injection molding, cast molding, etc.). The phase changeable fluid can be essentially any fluid that can be induced to solidify, or that spontaneously solidifies, into a solid capable of containing and/or transporting fluids contemplated for use in and with the fluidic network In one embodiment, the phase changeable fluid comprises a polymeric liquid or a liquid polymeric precursor (for example: a “prepolymer”). Suitable polymeric liquids can include, for example, thermoplastic polymers, thermoset polymers, waxes, metals, or mixtures or composites thereof heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, dodecyltrichlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of various structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65 °C. to about 75 °C for exposure times of, for example, about an hour. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures or channels from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, structures can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example Duffy, David C., J. Cooper McDonald, Olivier J. A. Schueller, and George M. Whitesides. “Rapid Prototyping of Microfluidic Systems in Poly(Dimethylsiloxane).” Analytical Chemistry 70, no. 23 (Dec. 1, 1998): 4974-84.

The device may comprise glass, silicon, silicon nitride, silicon oxide, quartz, metal, fused silica, mica, ceramics, aluminum, BK7 glass, linbo3, borofloat 33 glass, SD2 glass, 7740 pyrex glass, flexinity® glass, D263 glass, AF32 glass, mempax glass, B270 glass, AS87 ECO glass, foturan II glass, flexible glass, Schott UTG glass, flat planel display glass, KG2 glass, KG3 glass, NG11 glass, BG63 glass, EAGLE XG® glass, Astra™ glass, Lotus™ NXT glass, gallium arsenide, germanium, graphene, ITO, litao3, nitride, polysilicon, sapphire, silicon carbide, silicon on insulator, soda lime glass, InP, Polymers, Plastic, Polycarbonate (PC), PMMA, Acrylic, Polyethylene Terephthalate, Amorphous Copolyester (PETG), Polyvinyl Chloride (PVC), Liquid Silicone Rubber (LSR), Cyclic Olefin Copolymers (COC), Polyethylene (PE), Ionomer Resin, Polypropylene (PP), Fluorinated Ethylene Propylene (FEP), Styrene Methyl Methacrylate (SMMA), Styrene Acrylonitrile Resin (SAN), Polystyrene, Methyl Methacrylate Acrylonitrile Butadiene Styrene (MABS), acrylonitrile butadiene styrene (ABS), polylactic acid (PLA), polyethylene terephthalate glycol (PETG), Nylon, thermoplastic polyurethane (TPU), polyvinyl alcohol (PVA), high impact polystyrene (HIPS), Resins, Polyurethan Resins, Elastic Resins, ESD Resins, Ceramic Resins, Nylon 12, Nylon 11, TPU, Titanium, Stainless steel, Nickel alloys, gel, hydrogel, xerogel, colloidal gels, organolgels, thermoplastics, thermosetting plastics.

The device may comprise filter papers, parafilm, permanent maker inks, or wax.

The device may comprise a patternable material, in particular, a material comprising a patternable polymer, or a patternable organic polymer, or a patternable inorganic polymer, or a patternable gel. In some embodiments, the patternable material may comprise a photo-patternable material, or an electron-patternable material, or material capable of being embossed, or a material capable of being imprinted, or a material capable of being 3D printed, or a material capable of being dispensed from a dispensing system, or a material capable of being patterned with ion beam lithography, or a material capable of being patterned with soft-lithography, or a material capable of being patterned with colloidal lithography, or a material capable of being patterned by self-assembly methods, or a material capable of being patterned by area-selective deposition (ASD), or a material capable of being patterned by micro-contact printing, or a material capable of being patterned by casting, or a material capable of being patterned by lamination, or a material capable of being patterned by roll-to-roll processing, or a material capable of being patterned by microthermoforming, or a material capable of being patterned by micro-molding in capillaries (MIMIC), or a material capable of being patterned by capillary force lithography (CFL), or a material capable of being patterned by scanning probe lithography (SPL), or a material capable of being patterned by micro-cutting, or a material capable of being pattered by ultrasonic machine, or a material capable of being patterned by laser ablation, or a material capable of being patterned by plasma etching, or a material being capable of bio-templated assembly, or a material capable of being patterned by electro-discharge machining (EDM), or a material capable of being patterned by micro-electrochemical machining (ECM), or a material capable of being patterned by X-ray LIGA. For a non-limiting review of several non-photolithography methods of patterning materials, in particular polymers, see Qiu, Mingjun, Weiwei Du, Shangyu Zhou, Pengzhe Cai, Yingwu Luo, Xiaoxue Wang, Rong Yang, and Junjie Zhao. “Recent Progress in Non-Photolithographic Patterning of Polymer Thin Films.” Progress in Polymer Science 142 (July 2023): 101688, Acikgoz, Canet, Mark A. Hempenius, Jurriaan Huskens, and G. Julius Vancso. “Polymers in Conventional and Alternative Lithography for the Fabrication of Nanostructures.” European Polymer Journal 47, no. 11 (November 2011): 2033-52 and Scott, Simon, and Zulfiqur Ali. “Fabrication Methods for Microfluidic Devices: An Overview.” Micromachines 12, no. 3 (Mar. 18, 2021): 319, in particular 3D methods see Yazdi, Alireza Ahmadian, Adam Popma, William Wong, Tammy Nguyen, Yayue Pan, and Jie Xu. “3D Printing: An Emerging Tool for Novel Microfluidics and Lab-on-a-Chip Applications.” Microfluidics and Nanofluidics 20, no. 3 (March 2016): 50.

In some embodiments whereby the patternable material is a patternable polymer, the patternable polymer may comprise any of the previously listed polymer materials.

In some embodiments whereby the patternable material is a photo-patternable material, the photo-patternable material may comprise a photoresist. In some embodiments, the photoresist is a negative photoresist, or a positive photoresist, or a light-curable resist, or a UV-curable resist Examples of different photo-patternable photoresists include DNQ (diazonaphthoquinone) and novolac resin based resists, epoxy-based resists, Off-stoichiometry thiol-enes (OSTE) polymer resists, and hydrogen silsesquioxane (HSQ) based resists. In some embodiments the photo-patternable polymer is based on FOX-16 or its derivatives, or based on poly(methyl methacrylate) (PMMA) or its derivatives, or based on poly(methyl glutarimide) (PMGI) or its derivatives, or OSTE polymers or its derivatives, or based on Ma-N photoresists, or Shipley photoresists, or SPR photoresists. In some embodiments the photoresist comprises sensitivity for G-line, or I-line, or KrF, or ArF, or EUV, or electron beam. In some embodiments the photo-patternable polymer may comprise any Dupont SPR series of photoresists, or any Dupont MCPR series of photoresists, or any Dupont UV series of photoresists, or any Dupot UVN series of photoresist, or any Dupont LR series of photoresists, or any Dupont EPIC series of photoresists, or any Microchem AZ series of photoresist, or any Microchem TI series of photoresists, or any Microchem SU8 series of photoresists, or any Microchem Ordyl series of photoresists, of any microresist ma-N series of resists, or any microresist mr-DWL series of resists, or any microresist EpoCore series of resists, or any microresist EpoClad series of resists, or any microresist ma-P series of resists, or any microresist mr-P series of resists, or any microresist InkEpo series of resists, or any microchem InkOrmo series of resists, or any microchem SUEX series of resists, or any microresists ADEX series of resists, or any microresist UV series of resists, or any microresist microposit series of resists, or any microresist Ultra-I series of resists, or any microresist microposit LOL series of resists, or any microresist cyclotene series of resists, or any microresist intervia series of resists, or any microresist MX series of resist, or any microresist WBR series of resists, or any microresist SU-8 series of resists, or any microresist KMPR series of resists, or any microresist perminex series of resists, or any microresist KMSF series of resists, or any microresist LOR series of resist, or any Futurrex NR series of photoresists, or any Futurrex PR series of photoresists, or any Sumitomo PFI series of photoresist, or any Sumitomo PFM series of photoresists, or any Sumitomo PXi series of photoresists, or any Sumitomo NX series of photoresists, or any Sumitomo PEK series of photoresists, or any Sumitomo PAR series of photoresists, or any Tok TSMR series of photoresists, or any Tok THMR-iP series of resists, or any ToK TDMR-AR series of photoresists, or any ToK OFPR series of photoresists, or any Tok OEBR-CAP series of photoresists, or any ToK TFR-Di series of photoresists, or any Fujifilm OiR series of photoresists, or any Fujifilm GiR series of photoresists, or any Fujifilm HiPR series of photoresists, or any Fujifilm OiR series of photoresists, or any Fujifilm SC series of photoresists, or any Fujifilm IC series of photoresists, or any Fujifilm HNR series of photoresists, or any Fujifilm HR series of photoresists, or any Fujifilm GKR series of photoresists, or any Fujifilm GAR series of photoresists, or any Fujifilm FAIRS series of photoresists. In some embodiments, the photo-patternable polymer may comprise polymer resins, photoactive compounds, photoactivator compounds, photoinitiator compounds, detection additives, resist solvent, crosslinking agents, additives that assist the photoresist obtain the highest resolution, surfactants, quenchers, stabilizers, plasticizers, colorants, adhesive additives, surface leveling agents, or combinations thereof.

In some embodiments whereby the patternable material is an electron-patternable material, the electron-patternable material may comprise an electron beam resist. Examples of electron-patternable resists include any ZEP series of resists, or any HSQ and variants thereof, or any PMMA and variants thereof, or any polycarbonate and variants thereof, or any resists with increased sensitivity with the addition of halogenated acid generators, or any CSAR series of resists and variants thereof, or any AR series of resists, or any SML series of resists, or any microresist mr-EBL series of resists, or any microresist XR series of resists.

In some embodiments whereby the patternable material is an imprint capable material, the imprint capable material may comprise an imprint (or nano-imprint) resist. In some embodiments, the imprint resist comprises a UV curable polymer. In some embodiments, the imprint resist comprises a thermoset polymer. In some embodiments, the imprint resist comprises a thermoplastic polymer. In some embodiments, the imprint process may comprise nanoimprinting, electrochemical imprinting, laser assisted direct imprint, UV imprinting, ultrafast nanoimprint, roller imprint. Examples of imprint resists include any microresist mr-NIL series of resists, or any microresist mr-I series of resists, or any microresist SIPOL series of resists, or any microresist OrmoComp series of resists, or any microresist OrmoStamp series of resists, or any microresist OrmoClear series of resists, or any microresist OrmoCore series of resists, or any microresist OrmoClad series of resists, or any microresist mr-XNIL. series or resists, or any microresist KER series of resists, or any microresist mr-UVCur series of resists, or any nanonex NRX series of resists.

In some embodiments the patternable materials may be applied to the fluidic device by any method comprising spin coating, or lamination, or roller coating, or spray coating, or dip-coating, or dry-film attaching, or air-knife coating, or curtain coating, or wire-rod coating, or wire-bar coating, or gravure coating, or extraction coating, doctor blade coating, or dispensing from a dispensing system, or ink-jet printing, or jetting, or tape dispensing, or combinations there-of.

The device may comprise organic films that are chemically bonded or grown on a surface of the device. In some embodiments, the organic film comprises polymers. In some embodiments, the exact thickness of the organic film is controlled by the bonding or growth conditions of the film. In some embodiments, the organic film's growth is self-limiting. In some embodiments, the organic film's growth can be controlled by tuning the reaction time during growth, or by turning the number of growth cycles. In some embodiments, the organic film may comprise a hydrophobic surface property, or a hydrophilic surface property, or a positive surface charge, or a negative surface charge, or an anti-fouling surface property. In some embodiments the location(s) of the organic film in the fluidic device may be controlled by bonding or growing the organic film on a seed material, such that the organic film will only bond or grow on said seed material. In some embodiments, the seed material may comprise silicon oxide, or silicon nitride, or silicon, or glass, or quartz, or gold, or nickel, or platinum, or ITO, or silver, or a polymer, or a photo-patternable polymer, or an imprint-patternable polymer, or an embossing patternable polymer, or a dispensable polymer. In some embodiments, the seed material first has its surface chemically modified before bonding or growing the organic film. In some embodiments, the location(s) of an absence of organic film in the fluidic device is controlled at least in part by the location of a blocking material.

In some embodiments, the surface modification of a fluidic device comprises the modification of a polymerizable patternable material that has undergone partial or complete polymerization, with the degree of polymerization effected by factors including photoinitiation dose, lithographic masking, dose of a maskless lithography apparatus, wavelength dependent absorption of the light used to activate the photoinitiator, local environment created by fine features and neighbor effects, pre-bake thermal profile, post-bake thermal profile, development, aging, etching, plasma etching and quenching. In preferred embodiments, the modification of the patternable material employs chemical reactions that specifically modify the patternable material and do not modify the underlying substrate. In some embodiments, the above effects create a patternable material of non-uniform reactivity for the context of further chemical modification.

In some embodiments the surface modifications react in a substantially uniform and specific manner to modify substantially all of the underlying substrate or substantially of the patternable material applied to the substrate. In some embodiments, the surface modifications rely of further photoactivation of a developed or partially developed patternable material to enhance the reactivity of the remaining patternable material in local areas to add an additional level of spatial control of the deposition of subsequent surface modification. In some embodiments, selective application of plasma modifies the reactivity of the substrate and or the patternable material. In some embodiments the surface modifications react non-uniformly with a partially polymerized or partially degraded patternable material as prepared using the effects described above. In some embodiments, the degree of surface modification is further influenced by the diffusion of oxygen within the three dimensional structures during the process of surface modification.

In some embodiments, surface modifications are performed on a partially polymerized patternable material. In some embodiments, modifications utilize residual acid from superacid photoactivators and/or free radicals left over from radical polymerization. In some embodiments, radical polymerization is performed using a mixture of one or more acrylates and methacrylates, not limited to the following: poly(ethylene glycol), methyl ether acrylate, poly(2-hydroxyethyl methacrylate), isobornyl methacrylate, Lauryl methacrylate, 2-(Trimethylsilyloxy)ethyl methacrylate, N-Isopropylacrylamide, N,N′-Methylenebis(acrylamide), 2-Hydroxyethyl methacrylate, Propargyl acrylate, acrylamide, N,N-Dimethylacrylamide, 2-N-Morpholinoethyl methacrylate, Pentafluorophenyl methacrylate, dextran methacrylate, methacrylated gelatin, 2-Methacryloyloxyethyl phosphorylcholine, methacrylated hyaluronic acid, methacrylated collagen, methacrylated alginate, methyl methacrylate, 2,2,2-trifluoroethyl acrylate, butyl acrylate, propyl acrylate, N-hydroxyethyl acrylamide, 2-hydroxy-3-phenoxy-propyl acrylate, ter-butyl methacrylate, tert-butyl acrylate, sodium methacrylate, benzyl acrylate. In some embodiments, radical polymerization, including radical polymerization with the above monomers is carried out in a controlled living polymerization using one of the Reversible-deactivation radical polymerization methods including but not limited to Atom transfer radical polymerization (ATRP), Nitroxide-mediated polymerization (NMP), and Reversible addition-fragmentation chain transfer (RAFT).

In some embodiments, unreacted epoxy moieties from a patternable material can be opened and used for further reactions, including acylation reactions, including succinic anhydride and a-Bromoisobutyryl bromide. Furthermore, unreacted epoxy moieties react with primary amines under acid and base catalyzed conditions, and direct surface modifications of epoxy containing patternable materials are not limited to the addition of alkylamines including dodecylamine, octylamine, tert-Octylamine propylamine, cyclic aliphatic amines including cyclohexamine, fluorinated amines including 2,2,3,3,4,4,4-Heptafluorobutylamine, zwitterionic or charged amines including glutamatic acid, polar amines such as 2-(2-Aminoethoxy) ethanol, and secondary amines such as Dioctylamine.

In some embodiments, at least a portion of the surface of the fluidic device, or at least a portion of a fluidic feature is modified by a combination of two different reactions, applied one at a time or simultaneously, whereby the first reaction is the substrate for the second reaction. In such reactions, chemical amplification can be achieved under certain reaction conditions. In some embodiments, the first reaction is the surface initiated radical polymerization of the ATRP initiator (2-(2-Bromoisobutyryloxy) ethyl methacrylate), alone or as a mixture of different acrylates or methacrylates or bi or multifunctional crosslinking acrylates or methacrylates, and the second step is surface initiated Atom Transfer Radical Polymerization (siATRP) using an acrylate or methacrylate as described above, and employing one of the established methods of Copper catalyzed ATRP including activators generated by electron transfer (AGET), activators regenerated by electron transfer (ARGET), initiators for continuous activator regeneration (ICAR) and regeneration of Cu(I) from oxidation of Cu(0) in the presence of ligands including PMDETA, Me6TREN, TPMA, TMPA-NME2. In other embodiments, the first reaction is the thiol epoxy condensation of thiol containing ATRP reagents such as Bis[2-(2′-bromoisobutyryloxy)ethyl]disulfide and Bis[2-(2-bromoisobutyryloxy) undecyl] disulfide, followed by siATRP using acrylates or methacrylates as described above. In other embodiments, the surface modification proceeds by radical polymerization of one or more monomer species containing carboxyls, including acrylic acid and methacrylic acid, and the second reaction utilizes N-Ethyl-N0-(3-(dimethylamino)-propyl)carbodiimide hydrochloride (EDC) and N-Hydroxysuccinimide (NHS) to graft biomolecules onto the polymer, including commonly prepared proteins known for blocking ability or the ability to create a hydrogel or matrix including whole or partial Bovine Serum Albumen, Collagen, Gelatins, telocollagen, and tropoelastin. In other embodiments, the first reaction is radical polymerization of glycidyl methacrylate and the second reaction is the addition of primary amines present on proteins as described above with glycidyl ethers, or the addition of hyaluronic acid, dextran, or Carboxymethyl cellulose to the glycidyl ethers.

The device may comprise a combination of different materials that are mixed, bonded, laminated, layered, grown, adhered, patterned, joined, coated, deposited, evaporated, sputtered, melted, merged, or combination there-of.

### Porous Material

A “porous material” is any composition of solid, or semi-solid matter that is porous in nature such that there is exists a fluid or gas substance that can pass through said porous material. In some embodiments the porous material may comprise a gel, formed by cross-linking a gelling agent. In some embodiments the porous material may comprise an artificial gel, manufactured with either random, or controlled pore sizes. In some embodiments the porous material my comprise a solid state material with holes etched in random or deterministically. In some embodiments, the porous material may comprise a polymer The porous material may be fluidic device channel in which there are patterned physical obstacles that between them have openings, for example: a collection of pillars. The pillars may be of consistent, random, or distribution of sizes. The pillars may be arranged in a regular, planned, or random manner. The porous material may be a collection of packed beads or packed isolated objects, such that the space between the beads or objects provides for the porous nature. The beads or isolated objects may be of consistent, random, or distribution of sizes.

The packing can be regular or random. In some embodiments, the porous material may be a material that is grown, etched, or deposited, Plawsky, J. L., Kim, J. K., & Schubert, E. F. (2009). Engineered nanoporous and nanostructured films. Materials Today, 12(6), 36-45. The material may be organic, inorganic, or a combination there-of. In some embodiments, the porous material may comprise only a single pore. In some embodiments, the porous material may comprise at least one pore. In some embodiments, the porous material may comprise a plurality of pores. In some embodiments at least two pores are connected to each other, In some embodiments, at least two pores are independent of each other. In some embodiments the porous material comprises at least one pore with a minimum cross-sectional dimension in the range of 50 microns to 5 nm. In some embodiments, the porous materials comprises at least one pore with a cross-sectional profile that is approximately circular, or oval, or square, or rectangle, or triangular, or polygon, or star shaped, or non-connected line, or a connected line. The line may vary in width. For clarity, examples of non-connected lines include “I” shape, or “S” shape, or “H” shape, while examples of connected lines include “8” shape, or “P” shape, or “Q” shape.

### Gel

“Gels” are defined as a substantially dilute or porous system composed of a “gelling agent” that has been cross-linked (“gelled”). Non-limiting examples of gels include agarose, polyacrylamide, hydrogels Caló, E., & Khutoryanskiy, V. V. (2015). Biomedical applications of hydrogels: A review of patents and commercial products. European Polymer Journal, 65, 252-267, DNA gels Gac̆anin, J., Synatschke, C. V., & Weil, T. (2020, January 11). Biomedical Applications of DNA-Based Hydrogels. Advanced Functional Materials, Vol. 30, p. 1906253. In the context of this document, a gel and a semi-gel are equivalent, where-by a semi-gel is a gel with incomplete cross-linking and/or low concentration of the gelling agent. In some embodiments, the gel may comprise a photodegradable property. In some embodiments, the cross-linking of the gelling agent may comprise photo-activation.

### Environmental Condition

An “environmental condition” may comprise any property of physics, matter, chemistry that surrounds a bio-molecule that may impact said bio-molecule's physical state, thermo-dynamic state, chemical state, or reactivity to other reagents. The impact on the bio-molecule may be due to the presence of the environmental condition, or a change in the environmental condition. An environmental condition may comprise a temperature, a pressure, a humidity level, a pH, an ionic concentration, a flow rate or direction. An environmental condition may comprise a flux, polarization, intensity of a wavelength of light. An environmental condition may comprise a solution composition, for example a concentration of a certain reagent within a solution, or a ratio of certain reagents within a solution, or a salt composition used for a particular buffer An environmental condition may comprise an external force acting on a bio-molecule, for example a solution or air flow rate. An environmental condition may comprise thermal conductivity property, an electrical conductivity property, an optical opacity or transparency property. An environmental condition may comprise an electric or magnetic field. An environmental condition may comprise sound of a certain frequency or intensity. An environmental condition may comprise an ultrasonic wave of a certain frequency or intensity. An environmental condition may comprise a gravitation force, or an effective gravitational force. An environmental condition may comprise a vapor pressure. An environmental condition may comprise the composition of gases in an atmosphere. An environmental condition may comprise the composition of vapors in an atmosphere. An environmental condition may comprise the relative volatility of an atmosphere.

### Dispensing System

Used herein, a “dispensing system” or “dispenser” is an instrument, or a component of an instrument that is capable of dispensing a volume of liquid from a dispensing tip, nozzle, or orifice (herein, collectively referred to as “tip”) at a desired location in (x, y, z) space. In some embodiments the liquid is dispensed as a continuous stream. In some embodiments, the liquid is dispensed as a series of drops. The drop size may be 100 micro liters or less, 10 micro liters or less, 1 micro liters or less, 100 pico liters or less, 10 pico liters or less, 1 pico liters or less, 100 femto liters or less, 10 femto liters or less, 1 femto liter or less, 100 atto liters or less, 10 atto liters or less. In some embodiments, the tip is composed of a consumable pipette tip. In some embodiments, the dispenser tip is also capable of extracting solution from a target solution in (x, y, z) space, and so the dispenser is also an “extractor”. In some embodiments, the dispensing and extraction tips are different tips. In some embodiments, they are the same. In some embodiments, the tip is a micro-syringe, or the end of a capillary tube, or a nozzle. In some embodiments, the dispensing of liquid is controlled by air-displacement via a pressured air-line, or a syringe-pump moved via an electrical-mechanical system, such as a stepper motor.

In some embodiments, inkjet dispensers may be used. Inkjet printing includes continuous jet (CJ) and drop-on-demand jet (DODJ). The CJ based on the transducer, charging electrode and electric field can produce the droplet continuously, and the droplet location on a substrate can be determined by its charging density. There are several kinds of actuators for the DODJ device, including piezoelectric, thermal, solenoid, pneumatic, magnetostrictive and acoustic actuators. There are two actuation modes for the piezoelectric micro-jet devices in particular, including single actuation mode and hybrid actuation mode. The single actuation mode includes shear mode, squeeze mode, bend mode, push mode and needle-collision mode, while the hybrid actuation mode refers to electrohydrodynamic (EHD) assistant actuation. A detailed review of different inkjet technologies is provided by Li, H., Liu, J., Li, K., & Liu, Y. (2019). Piezoelectric micro-jet devices: A review. Sensors and Actuators, A: Physical, 297, 111552, and included here for reference in its entirety.

In some embodiments the dispenser consists of a contact probe capable of transporting and depositing a drop of solution by contact wetting. In some embodiments, extraction of drop from a surface is done by a contact probe making contact with said drop, and wetting the contact probe.

### Contact Probe System

Used herein, a “contact probe” system is an instrument, or a component within an instrument that is capable of positioning the point or tip of a contact probe within the desired location in (x, y, z) space with respect to a surface, preferably with nanometer position accuracy or better., and measuring a signal as a function of the xy, or xyz position. In the preferred embodiments, the contact probe is capable of measuring a signal based on its interaction with a physical object. In the preferred embodiment, the contact probe comprises part of a contact probe interrogation system, which itself is a type of interrogation system. In the preferred embodiments, the contact probe is a surface scanning probe, capable of generating a signal while the probe is physically moved in xyz space with respect to the surface by the instrument. Different types of contact probes include SPM (Scanning Probe Microscopy), AFM (Atomic Force Microscopy), HS-AFM (High Speed Atomic Force Microscopy), STM (Scanning Tunneling Microscopy), SPE (Scanning Probe Electrochemistry), CFM (Chemical Force Microscopy), LFM (lateral Force Microscopy), magnetic force microscopy (MFM), high frequency MFM, magneto-resistive sensitivity mapping (MSM), electric force microscopy (EFM), scanning capacitance microscopy (SCM), Scanning spreading resistance microscopy (SSRM), tunneling AFM and conductive AFM, contact AFM, non-contact AFM, dynamic contact AFM, tapping AFM, kelvin probe force microscopy (KPFM), piezo-response force microscopy (PFM), photothermal micro-spectroscopy, scanning gate microscopy (SGM), scanning quantum dot microscopy (SQDM), scanning voltage microscopy (SVM), force modulation microscopy (FMM), ballistic electron emission microscopy (BEEM), electrochemical scanning tunneling microscopy (ECSTM), scanning Hall probe microscopy (SHPM), spin polarized scanning tunneling microscopy (SPSM), photon scanning tunneling microscopy (PSTM) , scanning tunneling potentiometry (STP), synchrotron x-ray scanning tunneling microscopy (SXSTM), Scanning Probe Electrochemistry (SPE), scanning electrochemical microscopy (SECM), scanning ion-conductance microscopy (SICM), scanning vibrating electrode technique (SVET), scanning Kelvin probe (SKP), fluidic force microscopy (FluidFM), feature-oriented scanning probe microscopy (FOSPM), magnetic resonance force microscopy (MRFM), near-field scanning optical microscopy NSOM, scanning near-field optical microscopy (SNOM), scanning SQUID microscopy (SSM), scanning spreading resistance microscopy (SSRM), scanning thermal microscopy (SThM), scanning single-electron transistor microscopy (SSET), scanning thermo-ionic microscopy (STIM), charge gradient microscopy (CGM), and scanning resistive probe microscopy (SRPM). For a review of different Scanning Probe Microscopy systems, refer to Takahashi, Y., Kumatani, A., Shiku, H., & Matsue, T. (2017). Scanning Probe Microscopy for Nanoscale Electrochemical Imaging. Analytical Chemistry, 89(1), 342-357. For clarity, a contact probe need not necessarily make intimate physical contact with the sample, or any object, to measure a signal from said sample.

In some embodiments where the contact probe comprises an AFM system, the system can operate in a variety of different modes, and thus measure a variety of different signals, depending on the selection of the probe type, its mode of operation, and the probe's tip sharpness. Non limiting examples of different AFM modes include non-contact mode, contact mode, tapping mode, dry mode, wet mode, high-frequency mode, ultra-high frequency mode, force-modulation mode, conductive mode, magnetic mode, super-sharp tip mode, diamond tip mode, high-aspect ratio mode, electron beam deposited tip mode, and carbon-nano-tube tip mode. In some embodiments, the contact probe can operate in a dry environment, or a humid environment, or a liquid environment. In some embodiments, the point of the contact probe can be functionalized with chemical moieties, biological bodies, or affinity groups to enable biochemical interaction with the physical object being probed. For a review of various functionalization that have been demonstrated on contact probes, refer to Ebner, A., Wildling, L., & Gruber, H. J. (2019). Functionalization of AFM tips and supports for molecular recognition force spectroscopy and recognition imaging. In Methods in Molecular Biology (Vol. 1886). In some embodiments, the point of the contact probe may include a carbon nanotube, a nanorod, or a nanospike. In some embodiments, the tip of the contact probe may include a pore, or nanopore, that allows for a fluidic connection to a fluidic channel or fluidic chamber within the contact probe.

In AFM microscopy, the probe is attached to a spring-loaded or flexible cantilever that is in contact with the surface to be analyzed. Contact is made within the molecular force range (for example: within the range of interaction of Van der Waal forces). Within AFM, different modes of operation are possible, including contact mode, non-contact mode and TappingMode™.

In some embodiments, a contact probe interrogation system comprises multiple contact probes. In some embodiments, the collection of contact probes are all of the same type. In some embodiments, at least one contact probe within the set of contact probes is different. In some embodiments, the contact probes can all act independently with respect to their movement and orientation of their respective tips with their respective scanning surface. In some embodiments, at least two contact probes share at least one shared movement and orientation of their respective tips with respect to the scanning surface. For example: two contact probes may have independent z control, but share the same stage xy and rotation.

The disclosure is further understood through the following numbered embodiments. 1. A system comprising a surface and at least 10,000 non-overlapping nucleic acid molecules positioned on the surface. 2. The system of any previous embodiment such as 1, wherein the non-overlapping nucleic acid molecules are positioned on a portion of the surface of no greater than 1000 mm square. 3. The system of any previous embodiment such as 1, wherein the non-overlapping nucleic acid molecules are unmodified. 4. The system of any previous embodiment such as 1, further comprising overlapping nucleic acid molecules, wherein no more than 10% of the nucleic acid molecules on the surface are overlapping. 5. The system of any previous embodiment such as 1, wherein no more than 10% of the nucleic acid molecules overlap at least one adjacent nucleic acid molecule. 6. The system of any previous embodiment such as 5, wherein no more than 1% of the nucleic acid molecules overlap at least one adjacent nucleic acid molecule. 7. The system of any previous embodiment such as 1, wherein the nucleic acid molecules are unmodified. 8. The system of any previous embodiment such as 1, wherein the nucleic acid molecules are synthesized in vivo. 9. The system of any previous embodiment such as 1, wherein the nucleic acid molecules are naturally occurring. 10. The system of any previous embodiment such as 1, wherein the nucleic acid molecules comprise basepairs having a density of nucleic acid basepairs of at least 100Gb/mm2. 11. The system of any previous embodiment such as 1, wherein the at least 10,000 non-overlapping nucleic acid molecules have a median length of at least 100 kb. 12. The system of any previous embodiment such as 1, wherein the system comprises at least 100,000 nucleic acid molecules positioned on the surface. 13. The system of any previous embodiment such as 1, wherein the system comprises at least 500,000 nucleic acid molecules positioned on the surface. 14. The system of any previous embodiment such as 1, wherein the system comprises at least 1,000,000 nucleic acid molecules positioned on the surface. 15. The system of any previous embodiment such as 1-14, wherein the nucleic acid molecules comprise at least 1,000,000 base-pairs. 16. The system of any previous embodiment such as 1-14, wherein the nucleic acid molecules comprise chromosomes. 17. The system of any previous embodiment such as 16, wherein the chromosomes comprise metaphase condensed chromosomes. 18. The system of any previous embodiment such as 16, wherein the chromosomes comprise human host DNA 19. The system of any previous embodiment such as 18, wherein the chromosomes comprise fetal DNA. 20. The system of any previous embodiment such as 18, wherein the chromosomes comprise pathogen DNA. 21. The system of any previous embodiment such as 18, wherein the chromosomes comprise viral DNA. 22. The system of any previous embodiment such as 18, wherein the chromosomes comprise ecDNA. 23. The system of any previous embodiment such as 18, wherein at least some of the chromosomes are bound to at least one label. 24. The system of any previous embodiment such as 23, wherein the labels comprise fluorescent labels. 25. The system of any previous embodiment such as 23, wherein the labels comprise nucleic acid oligos. 26. The system of any previous embodiment such as 23, wherein the labels comprise epitopes. 27. The system of any previous embodiment such as 23-26, wherein the labels comprise a first label that identifies a first chromosomal segment. 28. The system of any previous embodiment such as 27, wherein the labels comprise a second label that identifies a second chromosomal segment. 29. The system of any previous embodiment such as 27, wherein the at least one label binds to regions of the molecule that comprises AT content. 30. The system of any previous embodiment such as 27, wherein the at least one label binds to regions of the molecule that comprises CG content. 31. The system of any previous embodiment such as 27, wherein the at least one label binds to regions of the molecule that comprises chromatin. 32. The system of any previous embodiment such as 27, wherein the at least one label binds to regions of the molecule that comprises telomeres. 33. The system of any previous embodiment such as 27, wherein the at least one label binds to regions of the molecule that comprises centromeres. 34. A system of any previous embodiment such as 27, wherein the at least one label binds to regions of the molecule that comprises a sequence. 35. The system of any previous embodiment such as 27, wherein the at least one label binds to regions of the molecule that comprises a gene. 36. The system of any previous embodiment such as 27, wherein the at least one label binds to regions of the molecule that comprises a regulatory element. 37. The system of any previous embodiment such as 27, wherein the at least one label binds to regions of the molecule that comprises a higher order structure. 38. The system of any previous embodiment such as 16-28, wherein the chromosomes are present in a proportion indicative of a fetal chromosomal number abnormality. 39. The system of any previous embodiment such as 16-28, wherein at least one chromosome of the chromosomes exhibits a chromosomal abnormality. 40. The system of any previous embodiment such as 39, wherein the chromosomal abnormality is present on no more than 1% of chromosomes on the surface. 41. The system of any previous embodiment such as 39, wherein the chromosomal abnormality is present on no more than 0.1% of chromosomes on the surface. 42. The system of any previous embodiment such as 39, wherein the chromosomal abnormality is present on no more than 100 of chromosomes on the surface. 43. The system of any previous embodiment such as 39, wherein the chromosomal abnormality is present on no more than 10 of chromosomes on the surface. 44. The system of any previous embodiment such as 39, wherein the chromosomal abnormality is present on no more than 1 of chromosomes on the surface. 45. The system of any previous embodiment such as 16-28, wherein at least one chromosome of the chromosomes is a viral chromosome. 46. The system of any previous embodiment such as 16-28, wherein at least one chromosome of the chromosomes is a bacterial chromosome. 47. The system of any previous embodiment such as 16-28, wherein at least one chromosome of the chromosomes is an archaeal chromosome. 48. The system of any previous embodiment such as 16-28, wherein at least one chromosome of the chromosomes is a eukaryotic pathogen chromosome. 49. The system of any previous embodiment such as 16-28, wherein at least one chromosome of the chromosomes is a transgenic chromosome. 50. The system of any previous embodiment such as 16-28, wherein at least one chromosome of the chromosomes comprises an engineered base. 51. The system of any previous embodiment such as 16-28, wherein at least one chromosome of the chromosomes comprises a CRISPR edited site. 52. The system of any previous embodiment such as 1, wherein the surface comprises fluidic features having a dimension configured to accommodate buman metaphase chromosomes in single file series. 53. The system of any previous embodiment such as 1-52 comprising an interrogation component. 54. The system of any previous embodiment such as 53, comprising an interrogation analysis component. 55. The system of any previous embodiment such as 54, comprising a signature identified on a subset of interrogated molecules. 56. The system of any previous embodiment such as 55, wherein the signature is associated with a disease. 57. The system of any previous embodiment such as 56, wherein the disease is cancer. 58. The system of any previous embodiment such as 55, wherein the signature is associated with a phenotype. 59. The system of any previous embodiment such as 55, wherein the signature is associated with an inherited trait. 60. The system of any previous embodiment such as 55, wherein the signature is associated with a mutation. 61. The system of any previous embodiment such as 55, wherein the signature is associated with a non-inherited trait. 62. The system of any previous embodiment such as 55, wherein the signature is associated with a variation. 63. The system of any previous embodiment such as 55, wherein the signature is associated with a structural variation. 64. The system of any previous embodiment such as 55, wherein the signature is associated with a large structural variation. 65. The system of any previous embodiment such as 55, wherein the signature is associated with a SNP. 66. The system of any previous embodiment such as 55, wherein the signature is associated with an organism. 67. The system of any previous embodiment such as 55, wherein the signature is associated with an infection organism. 68. The system of any previous embodiment such as 55, wherein the signature is associated with an invading organism. 69. The system of any previous embodiment such as 55, wherein the signature is associated with a symbiotic organism. 70. The system of any previous embodiment such as 55, wherein the signature is associated with a symbiotic relationship. 71. The system of any previous embodiment such as 55, wherein the signature is associated with a host organism. 72. The system of any previous embodiment such as 55, wherein the signature is associated with a parasitic organism. 73. The system of any previous embodiment such as 55, wherein the signature is associated with a metagenomic profile. 74. The system of any previous embodiment such as 55, wherein the signature is associated with a micro-organism. 75. The system of any previous embodiment such as 55, wherein the signature is associated with a type of chromosome. 76. The system of any previous embodiment such as 55, wherein the signature is associated with an ecDNA. 77. The system of any previous embodiment such as 55, wherein the signature is associated with a circular nucleic acid. 78. The system of any previous embodiment such as 55, wherein the signature is associated with a type of genomic information. 79. The system of any previous embodiment such as 55, wherein the signature is associated with a type of cell. 80. The system of any previous embodiment such as 79, wherein the cell type is a circulating tumor cell. 81. The system of any previous embodiment such as 55, wherein the signature comprises a banding pattern. 82. The system of any previous embodiment such as 55, wherein the signature comprises a label. 83. The system of any previous embodiment such as 55, wherein the signature comprises a physical map. 84. The system of any previous embodiment such as 55, wherein the signature comprises a physical dimension. 85. The system of any previous embodiment such as 55, wherein the signature comprises a physical mass. 86. The system of any previous embodiment such as 55, wherein the signature comprises a morphology. 87. The system of any previous embodiment such as 55, wherein the signature comprises a topology. 88. The system of any previous embodiment such as 55, wherein the signature comprises a chromosome arm length. 89. The system of any previous embodiment such as 55, wherein identification of a signature comprises a comparison to a reference. 90. The system of any previous embodiment such as 55, wherein identification of a signature comprises an alignment to a reference 91. The system of any previous embodiment such as 1, wherein the density of the molecules on said surface is at least one per 500 microns square. 92. The system of any previous embodiment such as 1, wherein the surface comprises a fluidic device. 93. The system of any previous embodiment such as 1, wherein the molecules are interrogated on said surface. 94. The system of any previous embodiment such as 93, wherein the rate of interrogation is at least 100 chromosomes per minute. 95. The system of any previous embodiment such as 1, wherein the molecules are positioned on the surface by a process that comprises receding meniscus. 96. The system of any previous embodiment such as 95, wherein the receding meniscus process comprises blade coating. 97. The system of any previous embodiment such as 95, wherein the receding meniscus process comprises combing. 98. The system of any previous embodiment such as 95, wherein the receding meniscus process comprises dip-coating. 99. The system of any previous embodiment such as 1, wherein the molecules are positioned on the surface by a process that comprises centripetal force. 100. The system of any previous embodiment such as 1, wherein the molecules are positioned on the surface by a process that comprises dispensing. 101. The system of any previous embodiment such as 1, wherein the molecules are positioned on the surface by a process that comprises a fluid flow. 102. The system of any previous embodiment such as 101, wherein the fluid flow comprises capillary flow. 103. The system of any previous embodiment such as 1, wherein the surface comprises an open fluidic device. 104. The system of any previous embodiment such as 103, wherein the open fluidic device comprises fluidic features. 105. The system of any previous embodiment such as 104, wherein the molecules are preferentially positioned within the fluidic features. 106. The system of any previous embodiment such as 105, wherein the fluidic features comprise a dimension suitably sized to accommodate a molecule of a certain type. 107. The system of any previous embodiment such as 106, wherein the fluidic features comprise at least a second dimension suitably sized to accommodate a molecule of a certain second type. 108. The system of any previous embodiment such as 104, wherein the fluidic features comprise topological elements. 109. The system of any previous embodiment such as 104, wherein the fluidic features comprise channels. 110. The system of any previous embodiment such as 109, wherein the channels comprise a dimension between 10 and 0.1 microns. 111. The system of any previous embodiment such as 109, wherein the channels comprise a dimension that is similar to the dimension of the molecules. 112. The system of any previous embodiment such as 109, wherein the molecules are preferentially positioned within the channels. 113. The system of any previous embodiment such as 110, wherein the dimension comprises a width. 114. The system of any previous embodiment such as 110, wherein the dimension comprises a length. 115. The system of any previous embodiment such as 110, wherein the dimension comprises a depth. 116. The system of any previous embodiment such as 110, wherein the dimension comprises a height. 117. The system of any previous embodiment such as 110, wherein the dimension comprises a cross-section. 118. The system of any previous embodiment such as 104, wherein the fluidic features comprise pillars. 119. The system of any previous embodiment such as 118, wherein the pillars comprise a dimension between 10 and 0.1 microns. 120. The system of any previous embodiment such as 118, wherein the pillars comprise a dimension that is similar to the dimension of the molecules. 121. The system of any previous embodiment such as 118, wherein the molecules are preferentially positioned within the pillars 122. The system of any previous embodiment such as 119, wherein the dimension comprises a separation distance to an adjacent pillar. 123. The system of any previous embodiment such as 119, wherein the dimension comprises a depth. 124. The system of any previous embodiment such as 119, wherein the dimension comprises a height. 125. The system of any previous embodiment such as 119, wherein the dimension comprises a width. 126. The system of any previous embodiment such as 119, wherein the dimension comprises a length. 127. The system of any previous embodiment such as 119, wherein the dimension comprises a cross-section. 128. The system of any previous embodiment such as 119, wherein the dimension comprises a diameter. 129. The system of any previous embodiment such as 104, wherein the fluidic features comprises a region with a surface property. 130. The system of any previous embodiment such as 129, wherein the molecules are preferentially positioned within the region. 131. The system of any previous embodiment such as 129, wherein the molecules are preferentially positioned outside of the region. 132. The system of any previous embodiment such as 129, wherein the surface property comprises hydrophobicity. 133. The system of any previous embodiment such as 129, wherein the surface property comprises hydrophilicity. 134. The system of any previous embodiment such as 129, wherein the surface property comprises anti-fouling capability. 135. The system of any previous embodiment such as 129, wherein the surface property comprises a roughness greater than 50 nm rms. 136. The system of any previous embodiment such as 129, wherein the surface property comprises a roughness less than 50 nm rms. 137. The system of any previous embodiment such as 104, wherein the fluidic features comprises a material. 138. The system of any previous embodiment such as 104, wherein the fluidic features comprises a film. 139. The system of any previous embodiment such as 104, wherein the fluidic features comprises a monolayer. 140. The system of any previous embodiment such as 104, wherein the fluidic features comprises a polymer. 141. The system of any previous embodiment such as 104, wherein the fluidic features comprise a fluidic channel having a porous roof or a fluidic chamber having a porous roof. 142. The system of any previous embodiment such as 141, wherein at least one molecule of the molecules can transit through the porous roof. 143. The system of any previous embodiment such as 141, wherein at least one molecule of the molecules cannot transit through the porous roof. 144. The system of any previous embodiment such as 104, wherein the molecules are preferentially positioned within regions of the fluidic features that are substantially high with respect to the surface. 145. The system of any previous embodiment such as 104, wherein the molecules are preferentially positioned within regions of the fluidic features that are substantially low with respect to the surface. 146. A method comprising interrogating the system of any previous embodiment such as 1-52. 147. The method of any previous embodiment such as 146, wherein at least one molecule is further processed. 148. The method of any previous embodiment such as 147, wherein the processing comprises depositing a substance on at least one molecule positioned on the surface. 149. The method of any previous embodiment such as 148, wherein the substance comprises a gel. 150. The method of any previous embodiment such as 148, wherein the substance comprises a liquid. 151. The method of any previous embodiment such as 148, wherein the substance comprises a solution. 152. The method of any previous embodiment such as 148, wherein the substance comprises a dissolved polymer. 153. The method of any previous embodiment such as 148, wherein the substance comprises at least one reagent. 154. The method of any previous embodiment such as 148, wherein the substance comprises at least one enzyme. 155. The method of any previous embodiment such as 147, wherein the processing comprises exposing at least one molecule to at least one reagent. 156. The method of any previous embodiment such as 147, wherein the processing comprises exposing at least one molecule to at least one enzyme. 157. The method of any previous embodiment such as 147, wherein the processing comprises exposing at least one molecule to at least one reagent. 158. The method of any previous embodiment such as 147, wherein the processing comprises exposing at least one molecule to at least one environmental condition. 159. The method of any previous embodiment such as 147, wherein the processing comprises exposing at least one molecule to at least one chemical reaction. 160. The method of any previous embodiment such as 147, wherein the processing comprises the binding of at least one body to the at least one molecule. 161. The method of any previous embodiment such as 147, wherein the processing comprises the de-naturing of at least one portion of the at least one molecule. 162. The method of any previous embodiment such as 147, wherein the processing comprises the cleaving at least one portion of the at least one molecule. 163. The method of any previous embodiment such as 147, wherein the processing comprises the nicking at least one portion of the at least one molecule. 164. The method of any previous embodiment such as 147, wherein the processing comprises a deposition system. 165. The method of any previous embodiment such as 147, wherein the processing comprises a contact probe system. 166. The method of any previous embodiment such as 165, wherein the contract probe system comprises an AFM. 167. A surface comprising fluidic features having a dimension configured to accommodate human metaphase chromosomes in single file series. 168. The surface of any previous embodiment such as 167, wherein the fluidic features comprise at least 100 parallel grooves. 169. The surface of any previous embodiment such as 167, wherein the fluidic features comprise at least 1,000 parallel grooves. 170. A method of enriching for condensed chromosomes comprising lysing a population of cells under conditions to produce a lysate comprising unlysed nuclei, and discarding nuclei from the lysate, thereby generating enriched condensed chromosomes from anucleate cells. 171. The method of any previous embodiment such as 170, wherein the anucleate cells are in M-phase and comprise condensed chromosomes. 172. The method of any previous embodiment such as 170 or 171, comprising culturing the population of cells in contact with growth factors that allow the cells to progress through at least one phase of a eukaryotic cell cycle prior to lysing. 173. The method of any previous embodiment such as 172, comprising culturing the population of cells in contact with a cell cycle progression inhibition prior to lysing r. 174. The method of any previous embodiment such as 173, wherein the cell cycle inhibitor blocks cell cycle progression at M-phase. 175. The method of any previous embodiment such as 174, wherein the cell cycle inhibitor comprises colcamid. 176. The method of any previous embodiment such as 172, comprising synchronizing cell cycle progression. 177. The method of any previous embodiment such as 172, wherein the culturing the population of cells in contact with a cell cycle progression inhibitor synchronizes cell cycle progression. 178. The method of any previous embodiment such as 170-177, wherein lysing comprises generating an osmotic gradient across cell membranes. 179. The method of any previous embodiment such as 170-177, wherein lysing comprises contacting the cells to an enzyme. 180. The method of any previous embodiment such as 170-177, wherein discarding nuclei from the lysate comprises centrifugation. 181. The method of any previous embodiment such as 170-177, wherein discarding nuclei from the lysate comprises passive sedimentation. 182. The method of any previous embodiment such as 170-177, comprising contacting condensed chromosomes from anucleate cells to a detergent. 183. The method of any previous embodiment such as 182, wherein the detergent coats nucleic acids. 184. The method of any previous embodiment such as 182, wherein the detergent coats chromatin. 185. The method of any previous embodiment such as 170 to 184, comprising contacting the enriched condensed chromosomes from anucleate cells to a deposition buffer. 186. The method of any previous embodiment such as 185, wherein the deposition buffer is volatile at room temperature. 187. The method of any previous embodiment such as 185, wherein the deposition buffer maintains integrity of enriched condensed chromosomes. 188. The method of any previous embodiment such as 185, wherein the deposition buffer comprises stabilizing salts. 189. The method of any previous embodiment such as 185, wherein the deposition buffer is acidic. 190. The method of any previous embodiment such as 190, wherein the deposition buffer has a pH of no greater than 4. 191. The method of any previous embodiment such as 190, wherein the deposition buffer has a pH of no greater than 3. 192.

The method of any previous embodiment such as 190, wherein the deposition buffer bas a pH of about 2. 193. The method of any previous embodiment such as 190, wherein the deposition buffer has a pH of 2. 194. The method of any previous embodiment such as 185 to 193, comprising depositing the enriched condensed chromosomes from anucleate cells onto a surface. 195. The method of any previous embodiment such as 194, wherein the surface comprises fluidic features having a dimension configured to accommodate human metaphase chromosomes in single file series. 196. The method of any previous embodiment such as 195, wherein the fluidic features comprise at least 100 parallel grooves. 197. The method of any previous embodiment such as 195, wherein the fluidic features comprise at least 1,000 parallel grooves. 198. The method of any previous embodiment such as 194 to 197, wherein the enriched condensed chromosomes from anucleate cells are deposited such that no more than 1% of chromosomes overlap. 199. A composition comprising intact chromosomes unbound by nuclei, colcamid, a detergent, a volatile solvent, at an acidic pH. 200. The composition of any previous embodiment such as 199, wherein the composition comprises at least 10,000 intact chromosomes. 201. The composition of any previous embodiment such as 199, wherein the composition comprises at least 1,000,000 intact chromosomes. 202. The composition of any previous embodiment such as 199, wherein the intact chromosomes comprise Histone H1 proteins at a native chromatin concentration. 203. The composition of any previous embodiment such as 199, wherein the intact chromosomes comprise condensing proteins at a native chromatin concentration. 204. The composition of any previous embodiment such as 199, wherein the intact chromosomes comprise condensin 2 proteins at a native chromatin concentration. 205. The composition of any previous embodiment such as 199, wherein the intact chromosomes preserve physical linkage information for at least 50% of chromosome length. 206. The composition of any previous embodiment such as 199, wherein the intact chromosomes preserve physical linkage information for at least 75% of chromosome length. 207. The composition of any previous embodiment such as 199, wherein the intact chromosomes preserve physical linkage information for at least 90% of chromosome length. 208. The composition of any previous embodiment such as 199, wherein the intact chromosomes preserve physical linkage information for at least 95% of chromosome length. 209. The composition of any previous embodiment such as 199, wherein the intact chromosomes preserve physical linkage information for at least 99% of chromosome length. 210. The composition of any previous embodiment such as 199, wherein the acidic pH is no greater than 4. 211. The composition of any previous embodiment such as 199, wherein the acidic pH is no greater than 3. 212. The composition of any previous embodiment such as 199, wherein the acidic pH is about 2. 213. The composition of any previous embodiment such as 199, wherein the acidic pH is 2. 214. A method of analyzing chromosomes in a sample, comprising depositing the chromosomes on a surface such that at least 10,000 chromosomes are deposited and no more than 1% of the chromosomes overlap, interrogating the surface comprising the chromosomes to create an image, and performing an automated identification of the chromosomes in the image. 215. The method of any previous embodiment such as 214, such that at least 100,000 chromosomes are deposited and no more than 1% of the chromosomes overlap. 216. The method of any previous embodiment such as 214, wherein the chromosomes comprise metaphase condensed chromosomes. 217 The method of any previous embodiment such as 216, wherein the chromosomes comprise human host DNA 218. The method of any previous embodiment such as 216, wherein the chromosomes comprise fetal DNA. 219. The method of any previous embodiment such as 216, wherein the chromosomes comprise pathogen DNA. 220. The method of any previous embodiment such as 216, wherein the chromosomes comprise viral DNA. 221. The method of any previous embodiment such as 216, wherein at least one of the chromosomes are bound to at least one label. 222. The method of any previous embodiment such as 221, wherein the labels comprise fluorescent labels. 223. The method of any previous embodiment such as 221, wherein the labels comprise nucleic acid oligos. 224. The method of any previous embodiment such as 221, wherein the labels comprise epitopes. 225. The method of any previous embodiment such as 221-224, wherein the labels comprise a first label that identifies a first chromosomal segment. 226. The method of any previous embodiment such as 225, wherein the labels comprise a second label that identifies a second chromosomal segment. 227. The method of any previous embodiment such as 214-226, wherein the chromosomes are present in a proportion indicative of a fetal chromosomal number abnormality. 228. The method of any previous embodiment such as 214-226, comprising identifying a fetal chromosomal number abnormality when a first chromosome population is present at an amount that differs substantially from that of at least one second chromosomal population. 229. The method of any previous embodiment such as 214-226, wherein at least one chromosome of the chromosomes exhibits a chromosomal abnormality. 230. The method of any previous embodiment such as 229, wherein the chromosomal abnormality comprises a structural variation, translocation, insertion, deletion, inversion or duplication. 231. The method of any previous embodiment such as 229, wherein the chromosomal abnormality is present on no more than 1% of chromosomes on the surface. 232. The method of any previous embodiment such as 229, wherein the chromosomal abnormality is present on no more than 0.1% of chromosomes on the surface. 233. The method of any previous embodiment such as 229, wherein the chromosomal abnormality is present on no more than 100 of chromosomes on the surface. 234. The method of any previous embodiment such as 229, wherein the chromosomal abnormality is present on no more than 10 of chromosomes on the surface. 235. The method of any previous embodiment such as 229, wherein the chromosomal abnormality is present on no more than 1 of chromosomes on the surface. 236. The method of any previous embodiment such as 214-226, wherein at least one chromosome of the chromosomes is a viral chromosome. 237. The method of any previous embodiment such as 214-226, comprising identifying a viral constituent of the sample. 238. The method of any previous embodiment such as 214-226, wherein at least one chromosome of the chromosomes is a bacterial chromosome. 239. The method of any previous embodiment such as 214-226, comprising identifying a bacterial constituent of the sample. 240. The method of any previous embodiment such as 214-226, wherein at least one chromosome of the chromosomes is an archaeal chromosome. 241. The method of any previous embodiment such as 214-226, comprising identifying an archacal constituent of the sample. 242. The method of any previous embodiment such as 214-226, wherein at least one chromosome of the chromosomes is a eukaryotic pathogen chromosome, 243. The method of any previous embodiment such as 214-226, comprising identifying an eukaryotic constituent of the sample. 244. The method of any previous embodiment such as 214-226, wherein at least one chromosome of the chromosomes is a transgenic chromosome. 245. The method of any previous embodiment such as 214-226, comprising identifying a transgenic chromosome in the sample. 246. The method of any previous embodiment such as 214-226, wherein at least one chromosome of the chromosomes comprises an engineered base. 247. The method of any previous embodiment such as 214-226, wherein at least one chromosome of the chromosomes comprises a CRISPR edited site. 248. The method of any previous embodiment such as 214-226, wherein at least two chromosomes are identified as being similar to each other, with said at least two chromosomes forming a first set that all share a first similarity property. 249. The method of any previous embodiment such as 248, wherein the similarity is determined at the at least two chromosomes having at least 90% identical genomic content. 250. The method of any previous embodiment such as 248, wherein the similarity is determined by an alignment the at least two chromosomes's physical mpa. 251. The method of any previous embodiment such as 248, wherein the similarity is determined by comparing the lengths of the arms with respect to the centromere on each of the at least two chromosomes. 252. The method of any previous embodiment such as 248, wherein the similarity is determined by the presence of at least one labelling body. 253. The method of any previous embodiment such as 248, wherein an in-silico model of said similar at least two chromosomes is generated. 254. The method of any previous embodiment such as 248, wherein at least two additional chromosomes are identified as similar to each other, forming a second set that all share a second similarity property, where none of the chromosomes in the first set belong to the second set. 255. The method of any previous embodiment such as 254, wherein a ratio of the number of chromosomes that have first similarity property in the originating sample compared to the number of chromosomes that have a second similarity property in the originating sample is estimated by the relative size of the first set to the second set. 256. The method of any previous embodiment such as 254, wherein a ratio of the number of cells that have first similarity property in the originating sample compared to the number of cells that have a second similarity property in the originating sample is estimated by the relative size of the first set to the second set. 257. The method of any previous embodiment such as 255-256, wherein the similarity property is chromosome number. 258. The method of any previous embodiment such as 255-256, wherein the similarity property is the presence of a genomic lesion. 259. The method of any previous embodiment such as 255-256, wherein the similarity property is the type of chromosome. 260. The method of any previous embodiment such as 255-256, wherein the similarity property is the morphology of chromosome 261. The method of any previous embodiment such as 255-256, wherein the similarity property is chromosome shape. 262. The method of any previous embodiment such as 255-256, wherein the similarity property is chromosome arm presence. 263. The method of any previous embodiment such as 255-256, wherein the similarity property is number of centromeres. 264. The method of any previous embodiment such as 255-256, wherein the similarity property is circularity. 265. The method of any previous embodiment such as 214-247, wherein the automated identification does not comprise human assessment of the image. 266. The method of any previous embodiment such as 214-247, wherein the automated identification is verified by human assessment of the image. 267. The method of any previous embodiment such as 214-247, wherein the automated identification is completed in no more than 1 hour 268. The method of any previous embodiment such as 214-247, wherein the automated identification is completed in no more than one day. 269. The method of any previous embodiment such as 214-268, wherein the sample is obtained from circulating patient blood. 270. The method of any previous embodiment such as 214-268, wherein the sample is obtained from cultured cells.

### EXAMPLES

### Example 1. Preparation of a Sample Enriched for Chromosomes and Combination With Deposition Buffer

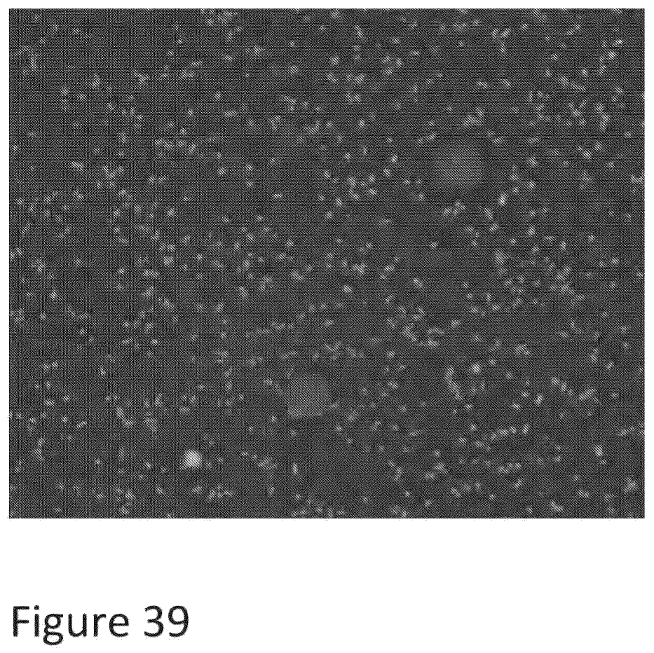

A T-75 flask was used to culture 10\hat{ }7 suspension cells of type K562 (ATCC CCL-243) with a volume of 20 ml and density of 5×10\hat{ }5 cells/ml. While cells were actively dividing, they were incubated with Colcemid to induce mitotic arrest and achieve a mitotic index of >20%, representing >2×10\hat{ }6 useful cells. Cells were harvested, hypotonically swollen in 75 mM KCI, and lysed in a citric acid/sucrose buffer containing a non-ionic detergent NP-40. Cell debris and nuclei were cleared by centrifugation at 200× g for 2 minutes and chromosomes were subsequently pelleted by centrifugation at 1600× g for 5 minutes. The chromosome pellet was washed by resuspending in a deposition buffer containing an intercalating fluorophore YOYO-1 and non-ionic detergent 0.2% Tween-20 and 0.2% Tween-80, and to prevent premature interaction of the chromosomes with the top of the substrate. The deposition buffer additionally contained volatile ions in the form of IM acetic acid. The chromosomes were again pelleted and resuspended in additional deposition buffer (FIG. 39).

### Example 2. Preparation of a Surface

The intended device lateral geometries that range from 1 to 10 micron are first defined using a CAD software program such that contact photomasks can be specified for order from a mask vendor. Once obtained, a glass borofloat wafer 0.5 mm thick is coated with a layer of photoresist spin coated over the surface, and then prepared for exposure according to the resist manufactures instructions. Operating a mask aligner in contact mode, the resist on the wafer is exposed through the mask to UV light, after which the resist is developed according to the instructions and chemicals recommended by the manufacturer to remove the exposed resist and expose the glass surface where the features will be formed. The exposed glass surface is then isotopically etched with a reactive ion etcher using a gas mixture include SF6 and CHF3, using the photoresist as an etch mask, and etching 1.5 micron deep. The photoresist etch mask is then removed with an oxygen plasma ash, and the etched glass substrate is then thoroughly washed in a heated mixture of water, ammonia, and hydrogen peroxide to remove any remaining organic material and facilitate particle removal from the surface. Next, the top protruding glass surface is modified with the addition of a hydrophobic silane monolayer. Silane treatment is performed by contact printing against a PDMS film that was previously submerged in a solvent of silane molecules, thus transferring the molecules to the regions between the channels via direct physical contact. The contact printing does not modify the inner walls and bottom of the channels, which due to their depressed topography, retain the glass's hydrophilic nature. After a 50 °C anneal for 1 hour, the device is ready for use.

### Example 3. Deposition of Chromosomes on the Patterned Substrate by Blade Coating

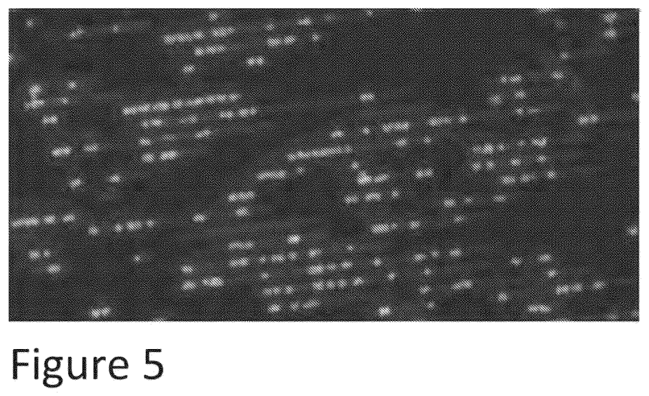

The mixture of chromosomes and deposition buffer from Example 1 was placed on the substrate from Example 2 and blade coated using an apparatus similar to that shown in FIG. 9. The footprint of the fluid was less than 25% of the substrate. A glass slide was brought into close proximity (<50 μm) of the substrate, at an angle of approximately 45 degrees to the substrate. The glass was translated across the substrate at a velocity of 4 μm/s using a computer-controlled step-motor stage in order to blade coat chromosomes onto the substrate. The device was interrogated using fluorescence microscopy (FIG. 5).

### Example 4. Interrogation of the Chromosomes by Fluorescence Microscopy.

The chromosomes that were deposited in Example 3 had been previously stained with an intercalating fluorophore. The substrate was capped with a coverglass and antifade mountant such as ProLong™ and imaged using a widefield epifluorescence microscope (FIG. 6).

### Example 5. Detection of Pathological Chromosomal Legions Using FISH

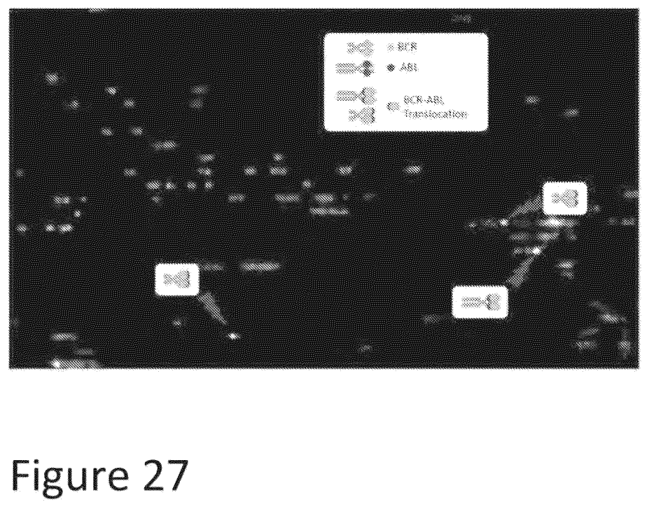

A sample substantially similar to Example 1 was stained with DAPI instead of an intercalating fluorophore and deposited using the method outlined in Example 3. The sample was dried and fixed with methanol and acetic acid. The cancer associated BCR/ABL fusion gene was identified by staining with a commercially available FISH kit from Empire Genomics, according to the manufacturer's instructions. The slide was imaged using multichannel epifluorescence wide-field microscopy (FIG. 27).

### Example 6. Deep Karyotyping to Detect Cancerous Cells in a Mostly Healthy Population of Cells

Culture a mixture of two cell populations, one with a normal chromosome 7, one with a translocation. Arrest both with colcemid, prepare, then dilute the translocation cell population into the healthy cell population at a defined ratio. Deposit the mixture, stain w/ Giemsa, image chromosomes, classify chromosomes, look for chromosomes with a translocation. Determine minimum fraction of cells that can be detected by this method.

### Example 7. Sequencing of Abnormal Chromosomes

Same as Example 6, but after identifying chromosomes with translocations, isolate the targeted chromosomes and use them to generate sequencing libraries. Non-limiting examples of different methods of picking up the chromosomes include laser-tweezer, solution absorption, solution suction, contact probe interaction, to name a few.

### Example 8. CRISPR-QC

Take a cell line and select a unique sequence that appears only once in the genome. Design a guide RNA and use a catalytically-inactive CRISPR-CAS9 in order to simulate the action of CRISPR. Deposit chromosomes on a substrate, then stain using the guide RNA together with a catalytically inactive CRISPR-CAS9 with a fluorescent label. Interrogate the chromosomes and classify the chromosomes by banding. Every time a CRISPR binding event is detected, determine whether it is on the correct chromosome.

### Example 9. Screening for Oncogenes on the Wrong Chromosomes

Deposit chromosomes, FISH for an onocogene as per Example 5, also band the chromosomes with G-banding with Trypsin to identify them. Optionally, put another FISH probe on the same chromosome where the oncogenes should be. Look for rare events where oncogenes are present in the wrong places. Repeat w/dilution of abnormal cells in normal cells to determine limit of detection.

### Example 10. Screening for ecDNA in Blood Cultures

Deposit chromosomes and image them. Count ratio of ecDNA to normal chromosomes. Isolate ecDNA if detected and desired.

### Example 11. Map the Length of Chromosome Bands

Deposit chromosomes that have been prepared using replication banding techniques, specifically the introduction of 5-ethynyl-2′-deoxyuridine upon release of Thymidine blockade. Deposit chromosomes and label all DNA non-specifically and bands specifically using cuAAC click chemistry Interrogate the chromosomes and for each chromosome construct a map of the observed bands, noting the boundaries of each band. Identify the chromosomes and for each band measure the observed extent of the band to calculate the observed degree of compaction.

### Example 12. Map the Length of Chromosome Bands and Identify Condensin II binding Sites

Prepare sample as Example 11, and further stain the sample using fluorescent antibodies directed against Condensin II, specifically the subunit NCAPH2. Image the antibodies, click-attached replication bands and non-specific stains. Catalog Condensin II binding sites and densities and correlate with band compaction data as an estimate of chromatin organization along the axis of the chromosome.

### Example 13. Double Sample Non-Invasive Prenatal Diagnosis (NIPT)

Non-invasive prenatal diagnosis (NIPT) is performed using a pair of maternal samples, one from before the pregnancy and one during the pregnancy. In both samples, chromosomes are elongated to the 500-650 band level and stained by Giemsa banding with Trypsin, then chromosomes are imaged, classified and screened for numerical and structural variations. The two samples are compared, looking for abnormalities present in the pregnancy sample that were not in the pre-pregnancy sample.

### Example 14 Integrated Fluidics Device For Cell Manipulation and Chromosome Deposition

Cells are grown in a culture flask and chromosomes are condensed by exposure to Colcemid for 3 hours, resulting in approximately 20% of cells to possess condensed metaphase chromosomes and dissolved nuclear membranes, though regardless of whether the cell is in metaphase, substantially all cells have intact cytoplasmic membranes. Cells are removed from the flask, concentrated and a portion of the cells from the flask are introduced to a fluidic device depicted in FIG. 33 and FIG. 34 by flow though inlet port 331, the flow depicted by an arrow 341. The cells 342 move into the cell chamber 333. Hypotonic expansion of the cells is achieved by application of hypotonic buffer by flow into the fluidic device, and the device is incubated at 37 C. After 30 minutes of hypotonic treatment, the device is cooled to 4 degrees and lysis buffer is flowed into the device (343) and then flow stops and cells are incubated for 30 mins during which time the lysis buffer breaks the external cell membrane but leaves the nuclei (344) of non-metaphase cells intact while liberating chromosomes (345) from metaphase cells that have a dissolved nuclear membrane. Additional lysis buffer is flowed into the device (346) at a low flow rate causing chromosomes to pass through the crude pillars (334) but not though (348) the fine pillars (336). Stabilization and wash agents are passed through the device to stabilize chromosomes and remove non-volatile buffer components. The outlet (332) is either plugged or allowed to flow backwards at a low flow rate while the inlet (331) is exposed to air, causing the buffer to evaporate through the inlet (349) and a meniscus to form (3410) that pulls the chromosomes along the channels (3411) and pins them to the surface of the interrogation chamber (335).

After drying the chromosomes are fixed with the application of Carnoys solution and dried again. Chromosomes are stained with Giemsa/Trypsin for G banding with Trypsin and chromosomes are imaged through the bottom of the device.

### Example 15. Deposition of Chromosomes on a Plain Substrate By Pipette Spotting and G Banding

The mixture of chromosomes and deposition buffer from Example 1 was deposited on a plain glass slide by simple pipette spotting as in FIG. 16 and allowed to dry. Dried chromosomes were washed with 3:1 methanol and acetic acid solution, dried, aged on a 90 degree C hotplate for 50 minutes, incubated in a trypsin solution for 45 s, quenched with FBS, then washed in PBS and stained in Giemsa stain, washed and dried. The chromosomes were imaged with a bright field microscope (FIGS. 35 & 38).

### Example 16. G-Banding of Chromosomes Deposited in Channels

Chromosomes immobilized in channels on a glass fluidic device were washed with 3:1 methanol and acetic acid, dried, aged on a 90 degree C hotplate for 50 minutes, incubated in a trypsin solution for 180s, quenched with FBS, then washed in 0.9% NaCl solution and stained in Giemsa stain for 3 minutes, washed and dried. The chromosomes were imaged with a 100×1.4 NA objective using brightfield microscope (FIG. 37).

### Example 17. Analysis of Chromosomes of Fetal Origin

A sample for analysis is fetal in origin and taken as a direct sample of amniotic fluid or chorionic villi. The cells are cultured in appropriate media, preferably AminoMAX media for amniotic fluid or Chang Medium D for chorionic villi or other media as described in Arsham et al. Chromosomes are condensed, cells are lysed and the sample is enriched as described herein. Chromosomes are deposited on the fluidic device and stained using Giemsa banding. One hundred thousand chromosomes are imaged and mapped to reference chromosomes. The statistical numbers of each chromosome are computed and compared with reference to assess the potential for aneuploidies. The lengths of banding patterns are additionally measured and compared with reference maps to assess the possibility of structural variations and rearrangements over each of a large number of chromosomes, and statistical analysis is used to assess the overall probability of structural rearrangements throughout the genome.

### Example 18. Detection of Integrated Viral DNA

Deposit chromosomes, apply FISH probes as per Example 5, but with multicolour FISH probes targeting both known sequences of viral DNA and control genes Optionally band the chromosomes with G-banding with Trypsin to identify them. Interrogate the chromosomes for banding patterns, morphology and detection of the FISH probes. Assess whether viral DNA is present in the chromosomes, and the location of each detected sites. Amass statistics on the frequency of integration and the number of unique integration sites detected.

### Example 19. Detection of Integrated HIV DNA

Conduct experiment and analysis as per Example 18, but with FISH probes generated against sequences corresponding to Human Immunodeficiency Virus (HIV).

What is claimed is as follows:

1. A system comprising a surface and at least 10,000 non-overlapping nucleic acid molecules positioned on the surface.

2. The system of claim 1, wherein the non-overlapping nucleic acid molecules are positioned on a portion of the surface of no greater than 1000 mm square.

3. The system of claim 1, wherein the non-overlapping nucleic acid molecules are unmodified.

4. The system of claim 1, further comprising overlapping nucleic acid molecules, wherein no more than 10% of the nucleic acid molecules on the surface are overlapping.

5. The system of claim 1, wherein no more than 10% of the nucleic acid molecules overlap at least one adjacent nucleic acid molecule.

6. The system of claim 5, wherein no more than 1% of the nucleic acid molecules overlap at least one adjacent nucleic acid molecule.

7. The system of claim 1, wherein the nucleic acid molecules are unmodified.

8. The system of claim 1, wherein the nucleic acid molecules are synthesized in vivo.

9. The system of claim 1, wherein the nucleic acid molecules are naturally occurring.

10. The system of claim 1, wherein the nucleic acid molecules comprise basepairs having a density of nucleic acid basepairs of at least 100Gb/mm2 .

11. The system of claim 1, wherein the at least 10,000 non-overlapping nucleic acid molecules have a median length of at least 100 kb.

12. The system of claim 1, wherein the system comprises at least 100,000 nucleic acid molecules positioned on the surface.

13. The system of claim 1, wherein the system comprises at least 500,000 nucleic acid molecules positioned on the surface.

14. The system of claim 1, wherein the system comprises at least 1,000,000 nucleic acid molecules positioned on the surface.

15. The system of any one of claims 1-14, wherein the nucleic acid molecules comprise at least 1,000,000 base-pairs.

16. The system of any one of claims 1-14, wherein the nucleic acid molecules comprise chromosomes.

17. The system of claim 16, wherein the chromosomes comprise metaphase condensed chromosomes.

18. The system of claim 16, wherein the chromosomes comprise human host DNA

19. The system of claim 18, wherein the chromosomes comprise fetal DNA.

20. The system of claim 18, wherein the chromosomes comprise pathogen DNA.

21. The system of claim 18, wherein the chromosomes comprise viral DNA.

22. The system of claim 18, wherein the chromosomes comprise ecDNA.

23. The system of claim 18, wherein at least some of the chromosomes are bound to at least one label.

24. The system of claim 23, wherein the labels comprise fluorescent labels.

25. The system of claim 23, wherein the labels comprise nucleic acid oligos.

26. The system of claim 23, wherein the labels comprise epitopes.

27. The system of any one of claims 23-26, wherein the labels comprise a first label that identifies a first chromosomal segment.

28. The system of claim 27, wherein the labels comprise a second label that identifies a second chromosomal segment.

29. The system of claim 27, wherein the at least one label binds to regions of the molecule that comprises AT content

30. The system of claim 27, wherein the at least one label binds to regions of the molecule that comprises CG content.

31. The system of claim 27, wherein the at least one label binds to regions of the molecule that comprises chromatin.

32. The system of claim 27, wherein the at least one label binds to regions of the molecule that comprises telomeres.

33. The system of claim 27, wherein the at least one label binds to regions of the molecule that comprises centromeres.

34. A system of claim 27, wherein the at least one label binds to regions of the molecule that comprises a sequence.

35. The system of claim 27, wherein the at least one label binds to regions of the molecule that comprises a gene.

36. The system of claim 27, wherein the at least one label binds to regions of the molecule that comprises a regulatory element.

37. The system of claim 27, wherein the at least one label binds to regions of the molecule that comprises a higher order structure.

38. The system of any one of claims 16-28, wherein the chromosomes are present in a proportion indicative of a fetal chromosomal number abnormality.

39. The system of any one of claims 16-28, wherein at least one chromosome of the chromosomes exhibits a chromosomal abnormality.

40. The system of claim 39, wherein the chromosomal abnormality is present on no more than 1% of chromosomes on the surface.

41. The system of claim 39, wherein the chromosomal abnormality is present on no more than 0.1% of chromosomes on the surface.

42. The system of claim 39, wherein the chromosomal abnormality is present on no more than 100 of chromosomes on the surface.

43. The system of claim 39, wherein the chromosomal abnormality is present on no more than 10 of chromosomes on the surface.

44. The system of claim 39, wherein the chromosomal abnormality is present on no more than 1 of chromosomes on the surface.

45. The system of any one of claims 16-28, wherein at least one chromosome of the chromosomes is a viral chromosome.

46. The system of any one of claims 16-28, wherein at least one chromosome of the chromosomes is a bacterial chromosome.

47. The system of any one of claims 16-28, wherein at least one chromosome of the chromosomes is an archaeal chromosome.

48. The system of any one of claims 16-28, wherein at least one chromosome of the chromosomes is a eukaryotic pathogen chromosome.

49. The system of any one of claims 16-28, wherein at least one chromosome of the chromosomes is a transgenic chromosome.

50. The system of any one of claims 16-28, wherein at least one chromosome of the chromosomes comprises an engineered base.

51. The system of any one of claims 16-28, wherein at least one chromosome of the chromosomes comprises a CRISPR edited site.

52. The system of claim 1, wherein the surface comprises fluidic features having a dimension configured to accommodate human metaphase chromosomes in single file series.

53. The system of any one of claims 1-52 comprising an interrogation component.

54. The system of claim 53, comprising an interrogation analysis component.

55. The system of claim 54, comprising a signature identified on a subset of interrogated molecules.

56. The system of claim 55, wherein the signature is associated with a disease.

57. The system of claim 56, wherein the disease is cancer.

58. The system of claim 55, wherein the signature is associated with a phenotype.

59. The system of claim 55, wherein the signature is associated with an inherited trait.

60. The system of claim 55, wherein the signature is associated with a mutation.

61. The system of claim 55, wherein the signature is associated with a non-inherited trait.

62. The system of claim 55, wherein the signature is associated with a variation.

63. The system of claim 55, wherein the signature is associated with a structural variation.

64. The system of claim 55, wherein the signature is associated with a large structural variation.

65. The system of claim 55, wherein the signature is associated with a SNP.

66. The system of claim 55, wherein the signature is associated with an organism.

67. The system of claim 55, wherein the signature is associated with an infection organism.

68. The system of claim 55, wherein the signature is associated with an invading organism.

69. The system of claim 55, wherein the signature is associated with a symbiotic organism.

70. The system of claim 55, wherein the signature is associated with a symbiotic relationship.

71. The system of claim 55, wherein the signature is associated with a host organism.

72. The system of claim 55, wherein the signature is associated with a parasitic organism.

73. The system of claim 55, wherein the signature is associated with a metagenomic profile.

74. The system of claim 55, wherein the signature is associated with a micro organism.

75. The system of claim 55, wherein the signature is associated with a type of chromosome.

76. The system of claim 55, wherein the signature is associated with an ecDNA.

77. The system of claim 55, wherein the signature is associated with a circular nucleic acid.

78. The system of claim 55, wherein the signature is associated with a type of genomic information.

79. The system of claim 55, wherein the signature is associated with a type of cell.

80. The system of claim 79, wherein the cell type is a circulating tumor cell.

81. The system of claim 55, wherein the signature comprises a banding pattern.

82. The system of claim 55, wherein the signature comprises a label.

83. The system of claim 55, wherein the signature comprises a physical map

84. The system of claim 55, wherein the signature comprises a physical dimension.

85. The system of claim 55, wherein the signature comprises a physical mass.

86. The system of claim 55, wherein the signature comprises a morphology

87. The system of claim 55, wherein the signature comprises a topology.

88. The system of claim 55, wherein the signature comprises a chromosome arm length.

89. The system of claim 55, wherein identification of a signature comprises a comparison to a reference.

90. The system of claim 55, wherein identification of a signature comprises an alignment to a reference

91. The system of claim 1, wherein the density of the molecules on said surface is at least one per 500 microns square.

92. The system of claim 1, wherein the surface comprises a fluidic device.

93. The system of claim 1, wherein the molecules are interrogated on said surface.

94. The system of claim 93, wherein the rate of interrogation is at least 100 chromosomes per minute.

95. The system of claim 1, wherein the molecules are positioned on the surface by a process that comprises receding meniscus.

96. The system of claim 95, wherein the receding meniscus process comprises blade coating.

97. The system of claim 95, wherein the receding meniscus process comprises combing.

98. The system of claim 95, wherein the receding meniscus process comprises dip-coating.

99. The system of claim 1, wherein the molecules are positioned on the surface by a process that comprises centripetal force.

100. The system of claim 1, wherein the molecules are positioned on the surface by a process that comprises dispensing.

101. The system of claim 1, wherein the molecules are positioned on the surface by a process that comprises a fluid flow.

102. The system of claim 101, wherein the fluid flow comprises capillary flow.

103. The system of claim 1, wherein the surface comprises an open fluidic device.

104. The system of claim 103, wherein the open fluidic device comprises fluidic features.

105. The system of claim 104, wherein the molecules are preferentially positioned within the fluidic features.

106. The system of claim 105, wherein the fluidic features comprise a dimension suitably sized to accommodate a molecule of a certain type.

107. The system of claim 106, wherein the fluidic features comprise at least a second dimension suitably sized to accommodate a molecule of a certain second type.

108. The system of claim 104, wherein the fluidic features comprise topological elements.

109. The system of claim 104, wherein the fluidic features comprise channels.

110. The system of claim 109, wherein the channels comprise a dimension between 10 and 0.1 microns.

111. The system of claim 109, wherein the channels comprise a dimension that is similar to the dimension of the molecules.

112. The system of claim 109, wherein the molecules are preferentially positioned within the channels.

113. The system of claim 110, wherein the dimension comprises a width.

114. The system of claim 110, wherein the dimension comprises a length.

115. The system of claim 110, wherein the dimension comprises a depth.

116. The system of claim 110, wherein the dimension comprises a height.

117. The system of claim 110, wherein the dimension comprises a cross-section.

118. The system of claim 104, wherein the fluidic features comprise pillars.

119. The system of claim 118, wherein the pillars comprise a dimension between 10 and 0.1 microns.

120. The system of claim 118, wherein the pillars comprise a dimension that is similar to the dimension of the molecules.

121. The system of claim 118, wherein the molecules are preferentially positioned within the pillars

122. The system of claim 119, wherein the dimension comprises a separation distance to an adjacent pillar.

123. The system of claim 119, wherein the dimension comprises a depth.

124. The system of claim 119, wherein the dimension comprises a height.

125. The system of claim 119, wherein the dimension comprises a width.

126. The system of claim 119, wherein the dimension comprises a length.

127. The system of claim 119, wherein the dimension comprises a cross-section.

128. The system of claim 119, wherein the dimension comprises a diameter.

129. The system of claim 104, wherein the fluidic features comprises a region with a surface property.

130. The system of claim 129, wherein the molecules are preferentially positioned within the region.

131. The system of claim 129, wherein the molecules are preferentially positioned outside of the region.

132. The system of claim 129, wherein the surface property comprises hydrophobicity.

133. The system of claim 129, wherein the surface property comprises hydrophilicity.

134. The system of claim 129, wherein the surface property comprises anti-fouling capability.

135. The system of claim 129, wherein the surface property comprises a roughness greater than 50 nm rms.

136. The system of claim 129, wherein the surface property comprises a roughness less than 50 nm rms.

137. The system of claim 104, wherein the fluidic features comprises a material.

138. The system of claim 104, wherein the fluidic features comprises a film.

139. The system of claim 104, wherein the fluidic features comprises a monolayer.

140. The system of claim 104, wherein the fluidic features comprises a polymer.

141. The system of claim 104, wherein the fluidic features comprise a fluidic channel having a porous roof or a fluidic chamber having a porous roof.

142. The system of claim 141, wherein at least one molecule of the molecules can transit through the porous roof.

143. The system of claim 141, wherein at least one molecule of the molecules cannot transit through the porous roof.

144. The system of claim 104, wherein the molecules are preferentially positioned within regions of the fluidic features that are substantially high with respect to the surface.

145. The system of claim 104, wherein the molecules are preferentially positioned within regions of the fluidic features that are substantially low with respect to the surface.

146. A method comprising interrogating the system of any one of claims 1-52.

147. The method of claim 146, wherein at least one molecule is further processed.

148. The method of claim 147, wherein the processing comprises depositing a substance on at least one molecule positioned on the surface.

149. The method of claim 148, wherein the substance comprises a gel.

150. The method of claim 148, wherein the substance comprises a liquid.

151. The method of claim 148, wherein the substance comprises a solution.

152. The method of claim 148, wherein the substance comprises a dissolved polymer.

153. The method of claim 148, wherein the substance comprises at least one reagent.

154. The method of claim 148, wherein the substance comprises at least one enzyme.

155. The method of claim 147, wherein the processing comprises exposing at least one molecule to at least one reagent.

156. The method of claim 147, wherein the processing comprises exposing at least one molecule to at least one enzyme.

157. The method of claim 147, wherein the processing comprises exposing at least one molecule to at least one reagent.

158. The method of claim 147, wherein the processing comprises exposing at least one molecule to at least one environmental condition.

159. The method of claim 147, wherein the processing comprises exposing at least one molecule to at least one chemical reaction.

160. The method of claim 147, wherein the processing comprises the binding of at least one body to the at least one molecule.

161. The method of claim 147, wherein the processing comprises the de-naturing of at least one portion of the at least one molecule.

162. The method of claim 147, wherein the processing comprises the cleaving at least one portion of the at least one molecule.

163. The method of claim 147, wherein the processing comprises the nicking at least one portion of the at least one molecule.

164. The method of claim 147, wherein the processing comprises a deposition system.

165. The method of claim 147, wherein the processing comprises a contact probe system.

166. The method of claim 165, wherein the contract probe system comprises an AFM.

167. A surface comprising fluidic features having a dimension configured to accommodate human metaphase chromosomes in single file series.

168. The surface of claim 167, wherein the fluidic features comprise at least 100 parallel grooves.

169. The surface of claim 167, wherein the fluidic features comprise at least 1,000 parallel grooves.

170. A method of enriching for condensed chromosomes comprising lysing a population of cells under conditions to produce a lysate comprising unlysed nuclei, and discarding nuclei from the lysate, thereby generating enriched condensed chromosomes from anucleate cells.

171. The method of claim 170, wherein the anucleate cells are in M-phase and comprise condensed chromosomes.

172. The method of claim 170 or 171, comprising culturing the population of cells in contact with growth factors that allow the cells to progress through at least one phase of a eukaryotic cell cycle prior to lysing.

173. The method of claim 172, comprising culturing the population of cells in contact with a cell cycle progression inhibition prior to lysing r.

174. The method of claim 173, wherein the cell cycle inhibitor blocks cell cycle progression at M-phase.

175. The method of claim 174, wherein the cell cycle inhibitor comprises colcamid.

176. The method of claim 172, comprising synchronizing cell cycle progression.

177. The method of claim 172, wherein the culturing the population of cells in contact with a cell cycle progression inhibitor synchronizes cell cycle progression.

178. The method of any one of claims 170-177, wherein lysing comprises generating an osmotic gradient across cell membranes.

179. The method of any one of claims 170-177, wherein lysing comprises contacting the cells to an enzyme.

180. The method of any one of claims 170-177, wherein discarding nuclei from the lysate comprises centrifugation.

181. The method of any one of claims 170-177, wherein discarding nuclei from the lysate comprises passive sedimentation.

182. The method of any one of claims 170-177, comprising contacting condensed chromosomes from anucleate cells to a detergent.

183. The method of claim 182, wherein the detergent coats nucleic acids.

184. The method of claim 182, wherein the detergent coats chromatin.

185. The method of any one of claims 170 to 184, comprising contacting the enriched condensed chromosomes from anucleate cells to a deposition buffer.

186. The method of claim 185, wherein the deposition buffer is volatile at room temperature.

187. The method of claim 185, wherein the deposition buffer maintains integrity of enriched condensed chromosomes.

188. The method of claim 185, wherein the deposition buffer comprises stabilizing salts.

189. The method of claim 185, wherein the deposition buffer is acidic.

190. The method of claim 190, wherein the deposition buffer has a pH of no greater than 4.

191. The method of claim 190, wherein the deposition buffer has a pH of no greater than 3.

192. The method of claim 190, wherein the deposition buffer has a pH of about 2.

193. The method of claim 190, wherein the deposition buffer has a pH of 2.

194. The method of any one of claims 185 to 193, comprising depositing the enriched condensed chromosomes from anucleate cells onto a surface.

195. The method of claim 194, wherein the surface comprises fluidic features having a dimension configured to accommodate human metaphase chromosomes in single file series.

196. The method of claim 195, wherein the fluidic features comprise at least 100 parallel grooves.

197. The method of claim 195, wherein the fluidic features comprise at least 1,000 parallel grooves.

198. The method of any one of claims 194 to 197, wherein the enriched condensed chromosomes from anucleate cells are deposited such that no more than 1% of chromosomes overlap.

199. A composition comprising intact chromosomes unbound by nuclei, colcamid, a detergent, a volatile solvent, at an acidic pH.

200. The composition of claim 199, wherein the composition comprises at least 10,000 intact chromosomes.

201. The composition of claim 199, wherein the composition comprises at least 1,000,000 intact chromosomes.

202. The composition of claim 199, wherein the intact chromosomes comprise Histone H1 proteins at a native chromatin concentration.

203. The composition of claim 199, wherein the intact chromosomes comprise condensin1 proteins at a native chromatin concentration.

204. The composition of claim 199, wherein the intact chromosomes comprise condensin2 proteins at a native chromatin concentration.

205. The composition of claim 199, wherein the intact chromosomes preserve physical linkage information for at least 50% of chromosome length.

206. The composition of claim 199, wherein the intact chromosomes preserve physical linkage information for at least 75% of chromosome length.

207. The composition of claim 199, wherein the intact chromosomes preserve physical linkage information for at least 90% of chromosome length.

208. The composition of claim 199, wherein the intact chromosomes preserve physical linkage information for at least 95% of chromosome length.

209. The composition of claim 199, wherein the intact chromosomes preserve physical linkage information for at least 99% of chromosome length.

210. The composition of claim 199, wherein the acidic pH is no greater than 4.

211. The composition of claim 199, wherein the acidic pH is no greater than 3.

212. The composition of claim 199, wherein the acidic pH is about 2.

213. The composition of claim 199, wherein the acidic pH is 2.

214. A method of analyzing chromosomes in a sample, comprising depositing the chromosomes on a surface such that at least 10,000 chromosomes are deposited and no more than 1% of the chromosomes overlap, interrogating the surface comprising the chromosomes to create an image, and performing an automated identification of the chromosomes in the image.

215. The method of claim 214, such that at least 100,000 chromosomes are deposited and no more than 1% of the chromosomes overlap.

216. The method of claim 214, wherein the chromosomes comprise metaphase condensed chromosomes.

217. The method of claim 216, wherein the chromosomes comprise human host DNA

218. The method of claim 216, wherein the chromosomes comprise fetal DNA.

219. The method of claim 216, wherein the chromosomes comprise pathogen DNA.

220. The method of claim 216, wherein the chromosomes comprise viral DNA.

221. The method of claim 216, wherein at least one of the chromosomes are bound to at least one label.

222. The method of claim 221, wherein the labels comprise fluorescent labels.

223. The method of claim 221, wherein the labels comprise nucleic acid oligos.

224. The method of claim 221, wherein the labels comprise epitopes.

225. The method of any one of claims 221-224, wherein the labels comprise a first label that identifies a first chromosomal segment.

226. The method of claim 225, wherein the labels comprise a second label that identifies a second chromosomal segment.

227. The method of any one of claims 214-226, wherein the chromosomes are present in a proportion indicative of a fetal chromosomal number abnormality.

228. The method of any one of claims 214-226, comprising identifying a fetal chromosomal number abnormality when a first chromosome population is present at an amount that differs substantially from that of at least one second chromosomal population.

229. The method of any one of claims 214-226, wherein at least one chromosome of the chromosomes exhibits a chromosomal abnormality.

230. The method of claim 229, wherein the chromosomal abnormality comprises a structural variation, translocation, insertion, deletion, inversion or duplication.

231. The method of claim 229, wherein the chromosomal abnormality is present on no more than 1% of chromosomes on the surface.

232. The method of claim 229, wherein the chromosomal abnormality is present on no more than 0.1% of chromosomes on the surface.

233. The method of claim 229, wherein the chromosomal abnormality is present on no more than 100 of chromosomes on the surface.

234. The method of claim 229, wherein the chromosomal abnormality is present on no more than 10 of chromosomes on the surface.

235. The method of claim 229, wherein the chromosomal abnormality is present on no more than 1 of chromosomes on the surface.

236. The method of any one of claims 214-226, wherein at least one chromosome of the chromosomes is a viral chromosome.

237. The method of any one of claims 214-226, comprising identifying a viral constituent of the sample.

238. The method of any one of claims 214-226, wherein at least one chromosome of the chromosomes is a bacterial chromosome.

239. The method of any one of claims 214-226, comprising identifying a bacterial constituent of the sample.

240. The method of any one of claims 214-226, wherein at least one chromosome of the chromosomes is an archaeal chromosome.

241. The method of any one of claims 214-226, comprising identifying an archaeal constituent of the sample.

242. The method of any one of claims 214-226, wherein at least one chromosome of the chromosomes is a eukaryotic pathogen chromosome,

243. The method of any one of claims 214-226, comprising identifying an eukaryotic constituent of the sample.

244. The method of any one of claims 214-226, wherein at least one chromosome of the chromosomes is a transgenic chromosome.

245. The method of any one of claims 214-226, comprising identifying a transgenic chromosome in the sample.

246. The method of any one of claims 214-226, wherein at least one chromosome of the chromosomes comprises an engineered base.

247. The method of any one of claims 214-226, wherein at least one chromosome of the chromosomes comprises a CRISPR edited site.

248. The method of any one of claims 214-226, wherein at least two chromosomes are identified as being similar to each other, with said at least two chromosomes forming a first set that all share a first similarity property.

249. The method of claim 248, wherein the similarity is determined at the at least two chromosomes having at least 90% identical genomic content.

250. The method of claim 248, wherein the similarity is determined by an alignment the at least two chromosomes's physical mpa.

251. The method of claim 248, wherein the similarity is determined by comparing the lengths of the arms with respect to the centromere on each of the at least two chromosomes.

252. The method of claim 248, wherein the similarity is determined by the presence of at least one labelling body.

253. The method of claim 248, wherein an in-silico model of said similar at least two chromosomes is generated.

254. The method of claim 248, wherein at least two additional chromosomes are identified as similar to each other, forming a second set that all share a second similarity property, where none of the chromosomes in the first set belong to the second set.

255. The method of claim 254, wherein a ratio of the number of chromosomes that have first similarity property in the originating sample compared to the number of chromosomes that have a second similarity property in the originating sample is estimated by the relative size of the first set to the second set.

256. The method of claim 254, wherein a ratio of the number of cells that have first similarity property in the originating sample compared to the number of cells that have a second similarity property in the originating sample is estimated by the relative size of the first set to the second set.

257. The method of any one of claims 255-256, wherein the similarity property is chromosome number.

258. The method of any one of claims 255-256, wherein the similarity property is the presence of a genomic lesion.

259. The method of any one of claims 255-256, wherein the similarity property is the type of chromosome.

260. The method of any one of claims 255-256, wherein the similarity property is the morphology of chromosome

261. The method of any one of claims 255-256, wherein the similarity property is chromosome shape.

262. The method of any one of claims 255-256, wherein the similarity property is chromosome arm presence.

263. The method of any one of claims 255-256, wherein the similarity property is number of centromeres.

264. The method of any one of claims 255-256, wherein the similarity property is circularity.

265. The method of any one of claims 214-247, wherein the automated identification does not comprise human assessment of the image.

266. The method of any one of claims 214-247, wherein the automated identification is verified by human assessment of the image.

267. The method of any one of claims 214-247, wherein the automated identification is completed in no more than 1 bour.

268. The method of any one of claims 214-247, wherein the automated identification is completed in no more than one day.

269. The method of any one of claims 214-268, wherein the sample is obtained from circulating patient blood.

270. The method of any one of claims 214-268, wherein the sample is obtained from cultured cells.
